SEQUENCE LISTING (33 sequences)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - DNA, 7570 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagttgggactgcgctggggcttcctcgttgctcttttaagaggtgtccagtgtcaggtgcaattggtggagtctgggggaggcgtggtccagcctgggaggtccctgagactctcctgtgcagcgtctggattcgccttcagtagatatggcatgcactgggtccgccaggctccaggcaaggggctggagtgggtggcagttatatggtatgatggaagtaataaatactatgcagactccgtgaagggccgattcaccatctccagagacaattccaagaacacgcagtatctgcaaatgaacagcctgagagccgaggacacggctgtgtattactgtgcgagaggcggtgacttcctctactactactattacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 7597 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggggtcaaccgccatcctcaccatggagttggggctgcgctgggttctcctcgttgctcttttaagaggtgtccagtgtcaggtgcagctggtggagtctgggggaggcgtggtccagcctgggaggtccctgagactctcctgtgcagcgtctggattcaccttcagtaactatgtcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagcctgagagccgaggacacggctgtgtattactgtgcgggtggatataactggaactacgagtaccactactacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 7579 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagttgggacttagctgggttttcctcgttgctcttttaagaggtgtccagtgtcaggtccagctggtggagtctgggggaggcgtggtccagcctgggaggtccctgagactctcctgtgcagcgtctggattcaccttcagtagctatggcatgcactgggtccgccaggctccaggcaaggggctggactgggtggcaattatttggcatgatggaagtaataaatactatgcagactccgtgaagggccgattcaccatctccagagacaattccaagaagacgctgtacctgcaaatgaacagtttgagagccgaggacacggctgtgtattactgtgcgagagcttgggcctatgactacggtgactatgaatactacttcggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 7558 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatcccaccatggggtcaaccgtcatcctcgccctcctcctggctgttctccaaggagtctgtgccgaggtgcagctggtgcagtctggagcagaggtgaaaaagcccggggagtctctgaagatctcctgtaagggttctggatacagctttaccagttactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacggatggcagcagctggcccctttgactactggggccagggaaccctggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 7576 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccaagcttggatctcaccatggagtttgggctgtgctggattttcctcgttgctcttttaagaggtgtccagtgtcaggtgcagctggtggagtctgggggaggcgtggtccagcctgggaggtccctgagactctcctgtgcagcctctggattcaccttcattagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagagtattagtgggagctttatattattataactactacgggatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 7561 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaggtaccggatctcaccatggagttggggctgagctgggttttcctcgttgctcttttaagaggtgtccagtgtcaggagcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttacatggcatgatggaagtaataaatactatgcagactccgtgaagggccgattcaccatctccagagacaactccaagaacacgctgtatctgcaaatgaacagcctgagagccgaggacacggctgtgtattactgtgcgagaggaggagtgggagcaacttactactactactacggtatggacgtctggggccaagggaccacggtcaccgtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctgggactcctgctgctctggctcccagataccagatgtgacatccagatgacccagtctccatcctccctgtctgcatctgtaggagacagagtcaccatcacttgccgggcgagtcagggcattagcaattatttagcctggtatcagcagaaaacagggaaagttcctaagttcctgatctatgaagcatccactttgcaatcaggggtcccatctcggttcagtggcggtggatctgggacagatttcactctcaccatcagcagcctgcagcctgaagatgttgcaacttattactgtcaaaattataacagtgccccattcactttcggccctgggaccaaagtggatatcaaacgaactgtggctgcaccctctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccccgctcagctcctggggctcctgctgctctgtttcccaggtgccagatgtgacatccagatgacccagtctccatcctcactgtctgcatctgtaggagacagagtcaccatcacttgtcgggcgagtcagggcattaccaattatttagcctggtttcagcagaaaccagggaaagcccctaagtcccttatctatgctgcatccagtttgcaaagtggggtcccatcaaagttcagcggcagtggatctgggacagatttcagtctcaccatcagcagcctgcagcctgaagattttgcaacttattactgccaacagtataatagttacccgatcaccttcggccaagggacacgactggagattaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 6082 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctggggctcctgctgctctgtttcccaggtgccagatgtgacatccagatgacccagtctccatcctcactgtctgcatctgtaggagacagagtcaccatcacttgtcgggcgagtcagggcattagccattatttagcctggtttcagcagaaaccagggaaagcccctaagtccctgatctatgctgcatccagtttgcaaagtggggtcccatcaaagttcagcggcagtggatctgggacagatttcactctcaccatcagcagcctacagcctgaagattttgcaacttattactgccaacagtataatagtttcccgctcactttcggcggagggaccaaggtggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 11 - DNA, 6085 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccccgctcagcttctcttccttctgctactctggctcccagataccactggaggaatagtgatgacgcagtctccagccaccctgtctgtgtctccaggggaaagagccaccctctcctgcaggaccagtcagagtattggctggaacttagcctggtaccaacagaaacctggccaggctcccaggctcctcatctatggtgcatcttccaggaccactggtatcccagccaggttcagtggcagtgggtctgggacagagttcactctcaccatcagcagcctgcagtctgaagattctgcagtttattactgtcagcattatgataactggcccatgtgcagttttggccaggggaccgagctggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 12 - DNA, 6097 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatgagggtccctgctcagctcctggggctgctaatgctctggatacctggatccagtgcagatattgtgatgacccagactccactctctctgtccgtcacccctggacagccggcctccatctcctgcaagtctagtcagagcctcctgcatagtgatggaaagacctttttgtattggtatctgcagaagccaggccagcctccacagctcctgatctatgaggtttccaaccggttctctggagtgccagataggttcagtggcagcgggtcagggacagatttcacactgaaaatcagccgggtggaggctgaggatgttgggctttattactgcatgcaaagtatacagcttccgctcactttcggcggagggaccaaggtggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 6094 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagaaagcttggatctcaccatggtgttgcagacccaggtcttcatttctctgttactctggatctctggtgcctacggggacatcgtgatgacccagtctccagactccctggctgtgtctctgggcgagagggccaccatcaactgcaagtccaaccagagtgtcttacacagctccaacaataagaactatttagcttggtaccagcagaaaccaggacagcctcctaaattgctcatttattgggcattcctccgggaatccggggtccctgaccgcttcagtggcagcgggtctgggacagatttcactctcaccatcagcagcctgcaggctgaagatgtggcagtttattactgtcaccaatattattctactttatatactttcggcggagggaccaaggtagagatcaaacgaacygtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 481 bp, Artificial Sequence
  Includes BamHI/Bg1II cloning junction, signal peptide, V region, portion of C region and 3′XbaI/NheI (heavy) or NheI (light) cloning junction
ggatctcaccatggagttgggactgcgctggggcttcctcgttgctcttttaagaggtgtccagtgtcaggtgcaattggtggagtctgggggaggcgtggtccagcctgggaggtccctgagactctcctgtgcagcgtctggattcgccttcagtagatatggcatgcactgggtccgccaggctccaggcaaggggctggagtgggtggcagttatatggtatgatggaagtaataaatactatgcagactccgtgaagggccgattcaccatctccagagacaattccaagaacacgcagtatctgcaaatgaacagcctgagagccgaggacacggctgtgtattactgtgcgagaggcggtgacttcctctactactactattacggtatggacgtctggggccaagggaccacggtcaccgtctcctcagcctccaccaagggcccatcggtcttccccctggcaccctctagc

SEQ ID NO: 15 - protein, 142 aa, Homo sapiens
MELGLRWGFLVALLRGVQCQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSS

SEQ ID NO: 16 - DNA, 463 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccctgctcagctcctgggactcctgctgctctggctcccagataccagatgtgacatccagatgacccagtctccatcctccctgtctgcatctgtaggagacagagtcaccatcacttgccgggcgagtcagggcattagcaattatttagcctggtatcagcagaaaacagggaaagttcctaagttcctgatctatgaagcatccactttgcaatcaggggtcccatctcggttcagtggcggtggatctgggacagatttcactctcaccatcagcagcctgcagcctgaagatgttgcaacttattactgtcaaaattataacagtgccccattcactttcggccctgggaccaaagtggatatcaaacgaactgtggctgcaccctctgtcttcatcttcccgccatctgatgagcagttgaaatctggaactgctagc

SEQ ID NO: 17 - protein, 127 aa, Homo sapiens
MRVPAQLLGLLLLWLPDTRCDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIK

SEQ ID NO: 18 - DNA, 508 bp, Artificial Sequence
  Includes BamHI/Bg1II cloning junction, signal peptide, V region, portion of C region and 3′XbaI/NheI (heavy) or NheI (light) cloning junction
ggatctcaccatggggtcaaccgccatcctcaccatggagttggggctgcgctgggttctcctcgttgctcttttaagaggtgtccagtgtcaggtgcagctggtggagtctgggggaggcgtggtccagcctgggaggtccctgagactctcctgtgcagcgtctggattcaccttcagtaactatgtcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagcctgagagccgaggacacggctgtgtattactgtgcgggtggatataactggaactacgagtaccactactacggtatggacgtctggggccaagggaccacggtcaccgtctcctcagcctccaccaagggcccatcggtcttccccctggcaccctctagc

SEQ ID NO: 19 - protein, 143 aa, Homo sapiens
MELGLRWVLLVALLRGVQCQVQLVESGGGVVQPGRSLRLSCAASGFTFSNYVMHWVRQAPGKGLEWVAIIWYDGSNKYYADSVKGRFTISRDNSKNTLYLQMNSLRAEDTAVYYCAGGYNWNYEYHYYGMDVWGQGTTVTVSS

SEQ ID NO: 20 - DNA, 463 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccccgctcagctcctggggctcctgctgctctgtttcccaggtgccagatgtgacatccagatgacccagtctccatcctcactgtctgcatctgtaggagacagagtcaccatcacttgtcgggcgagtcagggcattaccaattatttagcctggtttcagcagaaaccagggaaagcccctaagtcccttatctatgctgcatccagtttgcaaagtggggtcccatcaaagttcagcggcagtggatctgggacagatttcagtctcaccatcagcagcctgcagcctgaagattttgcaacttattactgccaacagtataatagttacccgatcaccttcggccaagggacacgactggagattaaacgaactgtggctgcaccatctgtcttcatcttcccgccatctgatgagcagttgaaatctggaactgctagc

SEQ ID NO: 21 - protein, 127 aa, Homo sapiens
MRVPAQLLGLLLLCFPGARCDIQMTQSPSSLSASVGDRVTITCRASQGITNYLAWFQQKPGKAPKSLIYAASSLQSGVPSKFSGSGSGTDFSLTISSLQPEDFATYYCQQYNSYPITFGQGTRLEIK

SEQ ID NO: 22 - DNA, 490 bp, Artificial Sequence
  Includes BamHI/Bg1II cloning junction, signal peptide, V region, portion of C region and 3′XbaI/NheI (heavy) or NheI (light) cloning junction
ggatctcaccatggagttgggacttagctgggttttcctcgttgctcttttaagaggtgtccagtgtcaggtccagctggtggagtctgggggaggcgtggtccagcctgggaggtccctgagactctcctgtgcagcgtctggattcaccttcagtagctatggcatgcactgggtccgccaggctccaggcaaggggctggactgggtggcaattatttggcatgatggaagtaataaatactatgcagactccgtgaagggccgattcaccatctccagagacaattccaagaagacgctgtacctgcaaatgaacagtttgagagccgaggacacggctgtgtattactgtgcgagagcttgggcctatgactacggtgactatgaatactacttcggtatggacgtctggggccaagggaccacggtcaccgtctcctcagcctccaccaagggcccatcggtcttccccctggcaccctctagc

SEQ ID NO: 23 - protein, 145 aa, Homo sapiens
MELGLSWVFLVALLRGVQCQVQLVESGGGVVQPGRSLRLSCAASGFTFSSYGMHWVRQAPGKGLDWVAIIWHDGSNKYYADSVKGRFTISRDNSKKTLYLQMNSLRAEDTAVYYCARAWAYDYGDYEYYFGMDVWGQGTTVTVSS

SEQ ID NO: 24 - DNA, 463 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccctgctcagctcctggggctcctgctgctctgtttcccaggtgccagatgtgacatccagatgacccagtctccatcctcactgtctgcatctgtaggagacagagtcaccatcacttgtcgggcgagtcagggcattagccattatttagcctggtttcagcagaaaccagggaaagcccctaagtccctgatctatgctgcatccagtttgcaaagtggggtcccatcaaagttcagcggcagtggatctgggacagatttcactctcaccatcagcagcctacagcctgaagattttgcaacttattactgccaacagtataatagtttcccgctcactttcggcggagggaccaaggtggagatcaaacgaactgtggctgcaccatctgtcttcatcttcccgccatctgatgagcagttgaaatctggaactgctagc

SEQ ID NO: 25 - protein, 127 aa, Homo sapiens
MRVPAQLLGLLLLCFPGARCDIQMTQSPSSLSASVGDRVTITCRASQGISHYLAWFQQKPGKAPKSLIYAASSLQSGVPSKFSGSGSGTDFTLTISSLQPEDFATYYCQQYNSFPLTFGGGTKVEIK

SEQ ID NO: 26 - DNA, 469 bp, Artificial Sequence
  Includes BamHI/Bg1II cloning junction, signal peptide, V region, portion of C region and 3′XbaI/NheI (heavy) or NheI (light) cloning junction
ggatcccaccatggggtcaaccgtcatcctcgccctcctcctggctgttctccaaggagtctgtgccgaggtgcagctggtgcagtctggagcagaggtgaaaaagcccggggagtctctgaagatctcctgtaagggttctggatacagctttaccagttactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacggatggcagcagctggcccctttgactactggggccagggaaccctggtcaccgtctcctcagcctccaccaagggcccatcggtcttccccctggcaccctctagc

SEQ ID NO: 27 - protein, 138 aa, Homo sapiens
MGSTVILALLLAVLQGVCAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARRMAAAGPFDYWGQGTLVTVSS

SEQ ID NO: 28 - DNA, 466 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccccgctcagcttctcttccttctgctactctggctcccagataccactggaggaatagtgatgacgcagtctccagccaccctgtctgtgtctccaggggaaagagccaccctctcctgcaggaccagtcagagtattggctggaacttagcctggtaccaacagaaacctggccaggctcccaggctcctcatctatggtgcatcttccaggaccactggtatcccagccaggttcagtggcagtgggtctgggacagagttcactctcaccatcagcagcctgcagtctgaagattctgcagtttattactgtcagcattatgataactggcccatgtgcagttttggccaggggaccgagctggagatcaaacgaactgtggctgcaccatctgtcttcatcttcccgccatctgatgagcagttgaaatctggaactgctagc

SEQ ID NO: 29 - protein, 128 aa, Homo sapiens
MRVPAQLLFLLLLWLPDTTGGIVMTQSPATLSVSPGERATLSCRTSQSIGWNLAWYQQKPGQAPRLLIYGASSRTTGIPARFSGSGSGTEFTLTISSLQSEDSAVYYCQHYDNWPMCSFGQGTELEIK

SEQ ID NO: 30 - DNA, 487 bp, Artificial Sequence
  Includes BamHI/Bg1II cloning junction, signal peptide, V region, portion of C region and 3′XbaI/NheI (heavy) or NheI (light) cloning junction
ggatctcaccatggagtttgggctgtgctggattttcctcgttgctcttttaagaggtgtccagtgtcaggtgcagctggtggagtctgggggaggcgtggtccagcctgggaggtccctgagactctcctgtgcagcctctggattcaccttcattagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagagtattagtgggagctttatattattataactactacgggatggacgtctggggccaagggaccacggtcaccgtctcctcagcctccaccaagggcccatcggtcttccccctggcaccctctagc

SEQ ID NO: 31 - protein, 144 aa, Homo sapiens
MEFGLCWIFLVALLRGVQCQVQLVESGGGVVQPGRSLRLSCAASGFTFISYGMHWVRQAPGKGLEWVAVISYDGSNKYYADSVKGRFTISRDNSKNTLYLQMNSLRAEDTAVYYCARVLVGALYYYNYYGMDVWGQGTTVTVSS

SEQ ID NO: 32 - DNA, 478 bp, Artificial Sequence
  Includes BamHI/Bg1II cloning junction, signal peptide, V region, portion of C region and 3′XbaI/NheI (heavy) or NheI (light) cloning junction
ggatctcaccatgagggtccctgctcagctcctggggctgctaatgctctggatacctggatccagtgcagatattgtgatgacccagactccactctctctgtccgtcacccctggacagccggcctccatctcctgcaagtctagtcagagcctcctgcatagtgatggaaagacctttttgtattggtatctgcagaagccaggccagcctccacagctcctgatctatgaggtttccaaccggttctctggagtgccagataggttcagtggcagcgggtcagggacagatttcacactgaaaatcagccgggtggaggctgaggatgttgggctttattactgcatgcaaagtatacagcttccgctcactttcggcggagggaccaaggtggagatcaaacgaactgtggctgcaccatctgtcttcatcttcccgccatctgatgagcagttgaaatctggaactgctagc

SEQ ID NO: 33 - protein, 132 aa, Homo sapiens
MRVPAQLLGLLMLWIPGSSADIVMTQTPLSLSVTPGQPASISCKSSQSLLHSDGKTFLYWYLQKPGQPPQLLIYEVSNRFSGVPDRFSGSGSGTDFTLKISRVEAEDVGLYYCMQSIQLPLTFGGGTKVEIK

(19) United States
(12) Patent Application Publication
(10) Pub. No.: US 2004/0161776 A1
(43) Pub. Date: Aug. 19, 2004

(54) PSMA formulations and uses thereof

(76) Inventors: Paul J. Maddon, Scarsdale, NY (US); Gerald P. Donovan, New York, NY (US); William C. Olson, Ossining, NY (US); Norbert Schulke, New City, NY (US); Jason Gardner, Ardsley, NY (US); Dangshe Ma, Millwood, NY (US)

Correspondence Address: Janice A. Vatland, Wolf, Greenfield & Sacks, P.C., 600 Atlantic Avenue, Boston, MA, 02210, US

(21) Appl. No.: 10/695,667

(22) Filed: Oct. 27, 2003

Related U.S. Application Data
(63) Continuation-in-part of application No. 10/395,894, filed on Mar. 21, 2003.
(63) Continuation-in-part of application No. PCT/US02/33944, filed on Oct. 23, 2002.
(60) Provisional application No. 60/335,215, filed on Oct. 23, 2001.
(60) Provisional application No. 60/362,747, filed on Mar. 7, 2002.
(60) Provisional application No. 60/412,618, filed on Sep. 20, 2002.

(51) Int. Cl. C12Q 1/68; G01N 33/574; C07H 21/04; C07K 14/705

(52) U.S. Cl. 435/6; 435/7.23; 435/69.1; 435/320.1; 435/325; 530/350; 536/23.5

(57) ABSTRACT

The invention includes stable multimeric, particularly dimeric, forms of PSMA protein, compositions and kits containing dimeric PSMA protein as well as methods of producing, purifying and using these compositions. Such methods include methods for eliciting or enhancing an immune reponse to cells expressing PSMA, including methods of producing antibodies to dimeric PSMA, as well as methods of treating cancer, such as prostate cancer.

### RELATED APPLICATIONS

This application is a continuation-in-part of U.S. nonprovisional application Ser. No. 10/395,894, filed on Mar. 21, 2003, which is a continuation-in-part of International application PCT/US02/33944 designating the United States, filed on Oct. 23, 2002, which claims the benefit under 35 U.S.C. § 119 of U.S. provisional application 60/335,215, filed Oct. 23, 2001, U.S. provisional application 60/362,747, filed Mar. 7, 2002, and U.S. provisional application 60/412,618, filed Sep. 20, 2002, each of which is incorporated herein by reference.

### FIELD OF THE INVENTION

This invention relates generally to the field of cancer associated polypeptides and formulations of and kits including these polypeptides. In particular, the invention relates, in part, to formulations of multimeric forms of PSMA proteins, particularly dimeric PSMA, and methods of their processing, purification, production and use.

### BACKGROUND OF THE INVENTION

Prostate cancer is the most prevalent type of cancer and the second leading cause of death from cancer in American men, with an estimated 179,000 cases and 37,000 deaths in 1999, (Landis, S. H. et al. CA Cancer J. Clin. 48:6-29 (1998)). The number of men diagnosed with prostate cancer is steadily increasing as a result of the increasing population of older men as well as a greater awareness of the disease leading to its earlier diagnosis (Parker et al., 1997, CA Cancer J. Clin. 47:5-280). The life time risk for men developing prostate cancer is about 1 in 5 for Caucasians, 1 in 6 for African Americans. High risk groups are represented by those with a positive family history of prostate cancer or African Americans.

Over a lifetime, more than ⅔ of the men diagnosed with prostate cancer die of the disease (Wingo et al., 1996, CA Cancer J. Clin. 46:113-25). Moreover, many patients who do not succumb to prostate cancer require continuous treatment to ameliorate symptoms such as pain, bleeding and urinary obstruction. Thus, prostate cancer also represents a major cause of suffering and increased health care expenditures.

Where prostate cancer is localized and the patient's life expectancy is 10 years or more, radical prostatectomy offers the best chance for eradication of the disease. Historically, the drawback of this procedure is that most cancers had spread beyond the bounds of the operation by the time they were detected. Patients with bulky, high-grade tumors are less likely to be successfully treated by radical prostatectomy.

Radiation therapy has also been widely used as an alternative to radical prostatectomy. Patients generally treated by radiation therapy are those who are older and less healthy and those with higher-grade, more clinically advanced tumors. Particularly preferred procedures are external-beam therapy which involves three dimensional, confocal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

For treatment of patients with locally advanced disease, hormonal therapy before or following radical prostatectomy or radiation therapy has been utilized. Hormonal therapy is the main form of treating men with disseminated prostate cancer. Orchiectomy reduces serum testosterone concentrations, while estrogen treatment is similarly beneficial. Diethylstilbestrol from estrogen is another useful hormonal therapy which has a disadvantage of causing cardiovascular toxicity. When gonadotropin-releasing hormone agonists are administered testosterone concentrations are ultimately reduced. Flutamide and other nonsteroidal, anti-androgen agents block binding of testosterone to its intracellular receptors. As a result, it blocks the effect of testosterone, increasing serum testosterone concentrations and allows patients to remain potent—a significant problem after radical prostatectomy and radiation treatments.

Cytotoxic chemotherapy is largely ineffective in treating prostate cancer. Its toxicity makes such therapy unsuitable for elderly patients. In addition, prostate cancer is relatively resistant to cytotoxic agents.

Relapsed or more advanced disease is also treated with anti-androgen therapy. Unfortunately, almost all tumors become hormone-resistant and progress rapidly in the absence of any effective therapy.

Accordingly, there is a need for effective therapeutics for prostate cancer which are not overwhelmingly toxic to normal tissues of a patient, and which are effective in selectively eliminating prostate cancer cells.

### SUMMARY OF THE INVENTION

The present invention relates, in part, to multimeric, particularly dimeric, forms of PSMA protein, compositions and kits containing dimeric PSMA protein as well as methods of producing, purifying, processing and using these compositions.

In one aspect compositions comprising multimeric forms of PSMA protein are provided. In some embodiments, these compositions contain isolated PSMA protein, at least 5% of which is in the form of PSMA protein multimer. In other embodiments at least 10%, 15%, 20%, 25%, 30%, 35%, 40%, 50%, 55%, 60%, 65%, 70%, 75%, 80%, 85%, 90%, 95% or more of the isolates PSMA protein is in the form of a PSMA protein multimer. In other embodiments the PSMA protein multimer is a PSMA protein dimer, wherein the PSMA protein dimer is formed by the covalent or non-covalent association of two PSMA proteins. In some embodiments the PSMA protein dimer is engineered to form a stable PSMA dimer through covalent bonds. In other embodiments the covalent bonds are disulfide bonds. Preferably, the PSMA protein dimer is associated in the same way as that of native PSMA dimer or is associated in such a way as to form at least one antigenic epitope that can be used to generate antibodies that recognize the native PSMA dimer. These antibodies, preferably, recognize the native PSMA dimer and not PSMA monomer or recognize the native PSMA dimer with greater specificity. In some embodiments of the invention the percent dimer can be calculated in terms of the number of PSMA protein molecules in the dimeric form versus the total number of PSMA protein (monomer, dimer or other multimer). In other embodiments the percent dimer can be calculated in terms of the number of PSMA dimers relative to the number of PSMA monomers, PSMA dimers and PSMA multimers.

In some embodiments the PSMA protein multimers comprise the full-length PSMA protein (SEQ ID NO: 1) or a fragment thereof. In other embodiments the PSMA protein multimer comprises the extracellular portion of PSMA (amino acids 44-750 of SEQ ID NO: 1) or a fragment thereof. In still other embodiments the PSMA protein multimer comprises the amino acids 58-750 of SEQ ID NO: 1 or a fragment thereof. In yet other embodiments the PSMA protein multimer comprises the amino acids 610-750 of SEQ ID NO: 1 or a fragment thereof. The fragments are capable of forming a PSMA multimer that can be used to generate antibodies that recognize PSMA, preferably native PSMA dimer. Typically, the PSMA multimers are homomultimers, meaning that the two or more PSMA molecules are the same. In other embodiments, the PSMA multimers are heteromultimers, whereby at least two of the PSMA proteins are not the same. In still other embodiments the PSMA proteins can be functionally equivalent proteins, whereby the PSMA protein is conservatively substituted.

In another aspect of the invention compositions comprising isolated multimeric PSMA protein, wherein the composition comprises less than 35% of a monomeric PSMA protein are provided. In still other embodiments the composition comprises less than 20% of the monomeric PSMA protein. In yet other embodiments the composition comprises less than 15% of the monomeric PSMA protein. In still other embodiments the composition comprises less than 5% of the monomeric PSMA protein. In some preferred embodiments the isolated multimeric PSMA protein is an isolated dimeric PSMA protein.

In some aspects of the invention, agents and compositions thereof that preserve or promote multimeric association of PSMA, particularly dimeric association, are provided. In some embodiments these agents include metal ions, salts, or pH adjusting agents. These agents that preserve or promote multimeric PSMA associations can do so individually or do so in combination. Therefore, in another aspect of the invention, a composition comprising PSMA protein multimers in conjunction with metal ion are provided. In some embodiments these compositions comprise at least 0.25 molar equivalents of metal ion to PSMA protein (total PSMA protein regardless of its form). In other embodiments at least 0.3, 0.4, 0.5, 0.6, 0.7, 0.8, 0.9, 1, 1.3, 1.5, 1.7, 2, 3, 4, 5, or more molar equivalents of metal ion to PSMA protein are present in the composition. In other embodiments the metal ion is in molar excess to PSMA protein. In some preferred embodiments the compositions provided are free of chelating agents.

In yet another aspect of the invention compositions comprising PSMA protein in a solution that promotes or preserves multimeric association of PSMA protein are provided. In some embodiments the solution that promotes or preserves multimeric association of PSMA protein is a solution that promotes or preserves dimeric association of PSMA protein. In other embodiments the solution that promotes or preserves dimeric association of PSMA protein has a pH that ranges from 4 to 8. In still other embodiments the solution that promotes or preserves dimeric association of PSMA protein has a pH that ranges from 5 to 7. Other embodiments include compositions wherein the solution that promotes or preserves dimeric association of PSMA protein has a pH that ranges from 5.5 to 7. In still other embodiments the solution that promotes or preserves dimeric association of PSMA protein has a pH of 6.

In still another aspect of the invention compositions comprising PSMA protein in a solution that promotes or preserves multimeric association of PSMA protein, wherein the solution comprises a salt, are provided. In some embodiments, the cationic component of the salt is sodium, potassium, ammonium, magnesium, calcium, zinc or a combination thereof, and the anionic component of the salt is chloride, sulfate, acetate or a combination thereof. In preferred embodiments the salt is sodium chloride, sodium sulfate, sodium acetate or ammonium sulfate. In some embodiments the salt is present at a concentration in the range of 50 mM to 2M. In other embodiments the salt is present at a concentration in the range of 100 mM to 300 mM. In still other embodiments the salt is present at a concentration of 150 mM.

In yet another aspect of the invention a composition comprising PSMA protein in a solution that promotes or preserves dimeric association of PSMA protein, wherein the solution comprises metal ions are provided. In some embodiments the metal ions are zinc ions, calcium ions, magnesium ions, cobalt ions, manganese ions or a combination thereof. In still other embodiments the metal ions are zinc ions and calcium ions. In yet other embodiments the zinc ions and calcium ions are present at a concentration in the range of 0.1 mM to 5 mM. In still other embodiments the zinc ions are present at a concentration that is lower than the concentration of the calcium ions. In some embodiments the zinc ions are present at a concentration of 0.1 mM and the calcium ions are present at a concentration of 1 mM. In other embodiments the metal ions are magnesium ions. In some of these embodiments the magnesium ions are present at a concentration in the range of 0.1 mM to 5 mM. In other embodiments the magnesium ions are present at a concentration of 0.5 mM. In a preferred embodiment the compositions are free of chelating agents.

In still a further aspect of the invention a composition comprising isolated PSMA protein in a solution that promotes or preserves dimeric association of PSMA protein wherein the solution comprises (a) 5 to 20 mM of sodium phosphate, sodium acetate or a combination thereof, (b) 100 to 300 mM sodium chloride or sodium sulfate, and (c) 0.1 to 2 mM of at least one metal ion is provided. In one embodiment the solution has a pH in the range of 4 to 8. In another embodiment the solution has a pH in a range of 5 to 7. In still another embodiment the solution has a pH in a range of 6 to 6.5. The metal ion in some embodiments is a zinc ion, calcium ion, magnesium ion, cobalt ion, manganese ion or a combination thereof.

In another aspect of the invention a composition comprising PSMA protein which also comprises an agent that promotes or preserves multimeric association, particularly dimeric association of PSMA protein, is provided, wherein the composition is stable when stored at −80° C. In other aspects of the invention the composition is stable when stored at −20° C. In still other aspects the composition is stable when stored at 4 C. In yet another aspect of the invention the composition is stable when stored at room temperature.

Another aspect of the invention provides a method of promoting or preserving dimeric association of PSMA protein in a solution by obtaining a solution of PSMA protein, and adjusting the pH to be in the range of 4 to 8. In some embodiments the pH is adjusted to be in the range of 5 to 7. In other embodiments the pH is adjusted to be in the range of 5.5 to 7. In yet other embodiments the pH is adjusted to be 6.

In another aspect of the invention a method of processing a PSMA protein by contacting the PSMA protein in a solution with a first agent that promotes or preserves dimeric association of PSMA protein in an amount effective to promote or preserve PSMA protein dimer formation is provided. In some embodiments the amount effective to promote or preserve PSMA protein dimer formation is enough to promote or maintain at least 5% of the PSMA protein in the solution in dimer form. In other embodiments at least 10%, 15%, 20%, 25%, 30%, 35%, 40%, 45%, 50%, 55%, 60%, 65%, 70%, 75%, 80%, 85%, 90%, 95% or more of the PSMA protein in the solution is in dimer form. The percentage of the dimer form of PSMA is calculated in terms of the total amount of the various forms of PSMA protein. In other words the percentage is calculated according to the number of PSMA dimers relative to the number of PSMA monomers, dimers and other multimers. In some embodiments the first agent that promotes or preserves dimeric association of PSMA protein is a salt, metal ion or a pH adjusting agent. The cationic components of the salt can include sodium, potassium, ammonium, magnesium, calcium, zinc or a combination thereof, while the anionic component of the salt can include chloride, sulfate, acetate or a combination thereof. In some embodiments the salt is sodium chloride, sodium sulfate, sodium acetate or ammonium sulfate. In other embodiments the salt is present at a concentration in the range of 50 mM to 2M. In still other embodiments the salt is present at a concentration in the range of 100 mM to 30 mM. In yet other embodiments the salt is present at a concentration of 150 mM. In some embodiments of the invention the method further includes combining the PSMA protein solution with an adjuvant or diluent. The adjuvant or diluent can be combined with the PSMA protein in an amount to dilute the salt concentration to 100 mM to 300 mM. In some embodiment the salt concentration is diluted to 150 mM. In certain embodiments this is done prior to administering the solution to a subject. In other embodiments the first agent is a metal ion and the metal ion is a zinc ion, calcium ion, magnesium ion, cobalt ion, manganese ion or a combination thereof. In some embodiments the metal ion is a combination of zinc ion and calcium ion. In still other embodiments the zinc ion and calcium ion are present at a concentration in the range of 0.1 mM to 5 mM. In yet other embodiments the zinc ion is present at a concentration that is lower than the concentration of the calcium ion. In still further embodiments the zinc ion is present at a concentration of 0.1 mM and the calcium ion is present at a concentration of 1 mM. In other embodiments the metal ion is a magnesium ion. In some of these embodiments the magnesium ion is present at a concentration in the range of 0.1 mM to 5 mM. In still other of these embodiments the magnesium ion is present at a concentration of 0.5 mM. In the embodiments where the first agent is a solution of a certain pH, the pH of the solution can be adjusted to be in the range of 4 to 8. In some embodiments the pH of the solution is adjusted to be in the range of 5 to 7. In still other embodiments the pH of the solution is adjusted to be in the range of 5.5 to 7. In yet other embodiments the pH of the solution is adjusted to be 6.

In some embodiments the method further comprises contacting the PSMA protein with a second agent that promotes or preserves dimeric association of PSMA protein, wherein the second agent is different than the first agent. A second agent that is different than the first agent includes agents that are of a different type or different class. The second agent, therefore, can be a metal ion, salt or pH adjusting agent. In some embodiments where the first agent is a metal ion the second agent can be a salt, pH adjusting agent or a solution with a certain pH. In other embodiments the first agent is a salt, and the second agent is a metal ion, pH adjusting agent or a solution with a certain pH. In still another embodiment the first agent is a pH adjusting agent or a solution with a certain pH and the second agent is a metal ion or a salt. In yet other embodiments the first agent can be a salt, metal ion, pH adjusting agent or a solution with a certain pH and the second agent can be of the same class but a different type within the same class of agents. For instance if the first agent is a salt such as sodium chloride, the second agent can also be a salt but a different type, e.g., ammonium sulfate.

In another aspect of the invention a method of purifying a sample containing PSMA protein by subjecting the sample containing PSMA to chromatography in the presence of an agent that preserves or promotes the dimeric association of PSMA is provided. In some embodiments the agent that promotes or preserves the dimeric association of PSMA is a metal ion, a salt or a solution with a pH in the range of 4 to 8 or a combination thereof. In a preferred embodiment the metal ion is a combination of calcium ion and magnesium ion. In one such embodiment the calcium ion and magnesium ion are each present at a concentration in the range of 0.1 mM to 5 mM. In a further embodiment the calcium ion and magnesium ion are present at a concentration of 1 mM and 0.5 mM, respectively. In other embodiments wherein the agent that promotes or preserves the dimeric association of PSMA is a salt, the salt is present at a concentration in the range of 50 mM to 2M. In some of these embodiments the salt is present at a concentration of 2M. In still other embodiments where the agent that promotes or preserves the dimeric association of PSMA is a solution with a pH in the range of 4 to 8, the pH of the solution is in the range of 5 to 7. In still other embodiments the pH of the solution is in the range of 6 to 7.5.

In other aspects of the invention a method of purifying a sample containing PSMA protein by applying the sample to a first column, washing the first column with a first wash solution containing salt and metal ions, and collecting the PSMA protein that elutes from the first column is provided. In some embodiments the salt is ammonium sulfate at a saturation of no more than 35% in the wash solution.

In embodiments of the invention the method further comprises dialyzing or diafiltering the eluted PSMA protein with a first salt solution at a pH in the range of 6 to 7.5 to yield a dialyzed or diafiltrated solution containing PSMA protein. In some of these embodiments the first salt solution has a salt concentration of at least 5 mM. In still other of these embodiments the first salt solution is a 10 mM sodium phosphate solution with a pH of 7.

In still other embodiments of the invention the method further comprises loading the eluted PSMA protein, dialyzed or diafiltrated solution containing PSMA protein onto a second column, washing the second column with a second salt solution, and collecting the PSMA eluted by the second salt solution. In some embodiments the second salt solution has a salt concentration of 100 mM to 2M. In certain of these embodiments the second salt solution is 2M sodium chloride in 10 mM sodium phosphate. In still other embodiments the second salt solution has a pH in the range of 6 to 7.5.

In yet another embodiment of the invention the method further comprises dialyzing or diafiltrating the PSMA eluted by the second salt solution with a metal ion solution, applying the dialyzed or diafiltrated PSMA eluted by the second salt solution onto a third column, washing the third column with a second wash solution containing salt and metal ions and collecting the PSMA eluted. In some of these embodiments the pH is maintained in the range of 6 to 7.5 through all of the purification steps.

In other embodiments the method further comprises separating the different forms of PSMA protein, wherein the different forms of PSMA protein are monomeric, dimeric or other multimeric forms of PSMA. In some of these embodiments the different forms of PSMA protein are separated by size exclusion chromatography.

In yet another aspect of the invention a method of identifying an agent which promotes or preserves dimeric association of PSMA protein by determining the amount of a form of PSMA protein in a sample prior to exposure to a candidate agent, exposing the sample to the candidate agent, determining the amount of the form of PSMA protein in the sample after the exposure, and comparing the amount of the form of PSMA protein in the sample prior to and after the exposure is provided. In some embodiments the form of PSMA protein is monomer or dimer. In other embodiments the form of PSMA can be another multimer form with three or more associated PSMA proteins.

In another aspect of the invention a method of treating a subject to elicit or enhance an immune response to cells in the subject expressing PSMA, comprising administering to the subject an effective amount of any of the compositions given herein is provided. In some embodiments the expressed PSMA is expressed on the cell surface. In other embodiments the method further comprises administering one or more booster doses of a composition comprising PSMA protein. In some of these embodiments the composition comprising PSMA protein is a composition of PSMA protein dimer. In still other embodiments the booster dose composition further comprises an adjuvant. In yet other embodiments the booster dose composition can be any of the compositions provided herein. In still other embodiments the composition is administered by intravenous, intramuscular, subcutaneous, parenteral, spinal, intradermal or epidermal administration. In this aspect of the invention the subject has cancer or is at risk of having cancer. In some embodiments the subject has also been treated for cancer. In some embodiments the cancer is a primary tumor or is metastatic cancer. In a preferred embodiment the subject has prostate cancer.

In another aspect of the invention a method of eliciting an immune response by administering to a subject an effective amount of any of the compositions provided is given. In some embodiments the method further comprises administering one or more booster doses of a composition comprising PSMA protein. In certain of these embodiments the composition comprising PSMA protein is a composition PSMA protein dimer. In still other embodiments the booster dose composition is any of the compositions given herein. In yet another embodiment the booster does compositions can also include an adjuvant.

In other aspects of the invention kits which contain any of the compositions provided and instructions for use are provided. In some aspects the kit contains a multimeric composition provided herein, an adjuvant and instructions for mixing. In other aspects the kit includes one of the compositions provided herein, a diluent and instructions for mixing. In some embodiments the composition is provided in a vial or ampoule with a septum or a syringe. In other embodiments the composition is in lyophilized form.

The compositions provided herein can further comprise a therapeutic agent (e.g., a cytokine, an anti-cancer agent, an adjuvant, etc.). In some embodiments the adjuvant is alum, monophosphoryl lipid A, a saponin, an immunostimulatory oligonucleotide, incomplete Freund's adjuvant, complete Freund's adjuvant, montanide, vitamin E, a water-in-oil emulsions prepared from a biodegradable oil, Quil A, a MPL and mycobacterial cell wall skeleton combination, ENHANZYN™, CRL-1005, L-121, alpha-galactosylceramide or a combination thereof.

In other embodiments the compositions provided can also include at least one buffer. Buffers include PBS (phosphate buffered saline), citric acid, sodium citrate, sodium acetate, acetic acid, sodium phosphate, phosphoric acid, sodium ascorbate, tartartic acid, maleic acid, glycine, sodium lactate, lactic acid, ascorbic acid, imidazole, sodium bicarbonate, carbonic acid, sodium succinate, succinic acid, histidine, sodium benzoate, benzoic acid and combinations thereof.

In some embodiments the compositions provided further include a free amino acid. These free amino acids can be naturally or non-naturally occurring. In some embodiments the free amino acids are non-acidic free amino acids. Examples of non-acidic free amino acids include glycine, proline, isoleucine, leucine, alanine, arginine and combinations thereof.

Compositions of PSMA protein multimers including a surfactant are also provided. Such surfactants include Tween20, Tween80, Triton X-100, dodecylmaltoside, cholic acid, CHAPS and combinations thereof.

Also provided are compositions of PSMA protein multimers that comprise a cryoprotectant, an antioxidant, a preservative or a combination thereof. Examples of cryoprotectants include a sugar, a polyol, an amino acid, a polymer, an inorganic salt, an organic salt, trimethylamine N-oxide, sarcosine, betaine, gamma-aminobutyric acid, octapine, alanopine, strombine, dimethylsulfoxide and ethanol. When the cryoprotectant is a sugar the sugar can be sucrose, lactose, glucose, trehalose or maltose. In other embodiments when the cryoprotectant is a polyol the polyol can be inositol, ethylene glycol, glycerol, sorbitol, xylitol, mannitol or 2-methyl-2,4-pentane-diol. When the cryoprotectant is an amino acid the amino acid can be Na glutamate, proline, alpha-alanine, beta-alanine, glycine, lysine-HCl or 4-hydroxyproline. When the cryoprotectant is a polymer the polymer can be polyethylene glycol, dextran or polyvinylpyrrolidone. When the cryoprotectant is an inorganic salt the cryoprotectant can be sodium sulfate, ammonium sulfate, potassium phosphate, magnesium sulfate or sodium fluoride. Finally, when the cryoprotectant is an organic salt the organic salt can be sodium acetate, sodium polyethylene, sodium caprylate, proprionate, lactate or succinate. Examples of antioxidants that are part of these composition in some embodiments include ascorbic acid, an ascorbic acid derivative, butylated hydroxy anisole, butylated hydroxy toluene, alkylgallate, dithiothreitol (DTT), sodium meta-bisulfite, sodium bisulfite, sodium dithionite, sodium thioglycollic acid, sodium formaldehyde sulfoxylate, tocopherol, a tocopherol derivative, monothioglycerol and sodium sulfite. Ascorbic acid derivatives, in some embodiments, include ascorbylpalmitate, ascorbylstearate, sodium ascorbate and calcium ascorbate, while tocopherol derivatives include d-alpha tocopherol, d-alpha tocopherol acetate, dl-alpha tocopherol acetate, d-alpha tocopherol succinate, beta tocopherol, delta tocopherol, gamma tocopherol and d-alpha tocopherol polyoxyethylene glycol 1000 succinate. Examples of preservatives present in the compositions in some embodiments include benzalkonium chloride, chlorobutanol, parabens, thimerosal, benzyl alcohol and phenol.

The composition in some embodiments are physiologically acceptable compositions.

The compositions provided are, in some embodiments, in liquid or lyophilized form.

In some other embodiments the compositions provided are sterile.

In other aspects of the invention pharmaceutical compositions are provided which contain any of the compositions provided herein and a pharmaceutically acceptable carrier.

The present invention also relates, in part, to antibodies or antigen-binding fragments thereof which specifically bind the extracellular domain of prostate specific membrane antigen (PSMA), compositions containing one or a combination of such antibodies or antigen-binding fragments thereof, hybridoma cell lines that produce the antibodies, and methods of using the antibodies or antigen-binding fragments thereof for cancer diagnosis and treatment.

According to one aspect of the invention, isolated antibodies or an antigen-binding fragments thereof are provided. The antibodies or fragments thereof specifically bind to an extracellular domain of prostate specific membrane antigen (PSMA), and competitively inhibit the specific binding of a second antibody to its target epitope on PSMA. In a second aspect of the invention, isolated antibodies or antigen-binding fragments thereof are provided which specifically bind to an epitope on prostate specific membrane antigen (PSMA) defined by a second antibody. In each of the forgoing aspects of the invention, the second antibody is selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, Abgenix 4.152.1, and antibodies comprising (a) a heavy chain encoded by a nucleic acid molecule comprising the coding region or regions of a nucleotide sequence selected from the group consisting of nucleotide sequences set forth as SEQ ID NOs: 2-7, and (b) a light chain encoded by a nucleic acid molecule comprising the coding region or regions of a nucleotide sequence selected from the group consisting of nucleotide sequences set forth as SEQ ID NOs: 8-13.

In certain embodiments, the antibody or antigen-binding fragment thereof is selected from the group consisting of PSMA 3.7, PSMA 3.8, PSMA 3.9, PSMA 3.11 PSMA 5.4, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, and Abgenix 4.152.1. In other embodiments, the antibody or antigen-binding fragment thereof is selected from the group consisting of antibodies comprising (a) a heavy chain encoded by a nucleic acid molecule comprising the coding region or regions of a nucleotide sequence selected from the group consisting of nucleotide sequences set forth as SEQ ID NOs: 2-7, and (b) a light chain encoded by a nucleic acid molecule comprising the coding region or regions of a nucleotide sequence selected from the group consisting of nucleotide sequences set forth as SEQ ID NOs: 8-13, and antigen-binding fragments thereof.

In further embodiments, the antibody or antigen-binding fragments thereof is encoded by a nucleic acid molecule comprising a nucleotide sequence that is at least about 90% identical to the nucleotide sequence encoding the foregoing antibodies, preferably at least about 95% identical, more preferably at least about 97% identical, still more preferably at least about 98% identical, and most preferably is at least about 99% identical.

In some embodiments of the foregoing aspects, antigen-binding fragments of the isolated antibodies are provided. The antigen-binding fragments include (a) a heavy chain variable region encoded by a nucleic acid molecule comprising the coding regions or regions of a nucleotide sequence selected from the group consisting of nucleotide sequences set forth as: SEQ ID NOs: 14, 18, 22, 26 and 30, and (b) a light chain variable region encoded by a nucleic acid molecule comprising the coding region or region of a nucleotide sequence selected from the group consisting of nucleotide sequences set forth as: SEQ ID NOs: 16, 20, 24, 28 and 32. In other embodiments, the antigen-binding fragment includes (a) a heavy chain variable region comprising an amino acid sequence selected from the group consisting of amino acid sequences set forth as: SEQ ID NOs: 15, 19, 23, 27 and 31, and (b) a light chain variable region comprising an amino acid sequence selected from the group consisting of nucleotide sequences set forth as: SEQ ID NOs: 17, 21, 25, 29 and 33.

In a further embodiments of the invention, isolated antigen-binding fragments of antibodies, which include a CDR of the foregoing antigen-binding fragments are provided. Preferably the CDR is CDR3.

According another aspect of the invention, expression vectors including an isolated nucleic acid molecule encoding the foregoing isolated antibodies or antigen-binding fragments is provided. Host cells transformed or transfected by these expression vectors also are provided.

In certain embodiments, the antibody or antigen-binding fragment thereof is selected for its ability to bind live cells, such as a tumor cell or a prostate cell, preferably LNCaP cells. In other embodiments, the antibody or antigen-binding fragment thereof mediates cytolysis of cells expressing PSMA. Preferably cytolysis of cells expressing PSMA is mediated by effector cells or is complement mediated in the presence of effector cells.

In other embodiments, the antibody or antigen-binding fragment thereof inhibits the growth of cells expressing PSMA. Preferably the antibody or antigen-binding fragment thereof does not require cell lysis to bind to the extracellular domain of PSMA.

In further embodiments, the antibody or antigen-binding fragment thereof is selected from the group consisting of IgG1, IgG2, IgG3, IgG4, IgM, IgA1, IgA2, IgAsec, IgD, IgE or has immunoglobulin constant and/or variable domain of IgG1, IgG2, IgG3, IgG4, IgM, IgA1, IgA2, IgAsec, IgD or IgE. In other embodiments, the antibody is a bispecific or multispecific antibody.

In still other embodiments, the antibody is a recombinant antibody, a polyclonal antibody, a monoclonal antibody, a humanized antibody or a chimeric antibody, or a mixture of these. In particularly preferred embodiments, the antibody is a human antibody, e.g., a monoclonal antibody, polyclonal antibody or a mixture of monoclonal and polyclonal antibodies. In still other embodiments, the antibody is a bispecific or multispecific antibody.

Preferred antigen-binding fragments include a Fab fragment, a F(ab′)₂ fragment, and a Fv fragment CDR3.

In further embodiments, the isolated antibody or antigen-binding fragment is a monoclonal antibody produced by a hybridoma cell line selected from the group consisting of PSMA 3.7 (PTA-3257), PSMA 3.8, PSMA 3.9 (PTA-3258), PSMA 3.11 (PTA-3269), PSMA 5.4 (PTA-3268), PSMA 7.1 (PTA-3292), PSMA 7.3 (PTA-3293), PSMA 10.3 (PTA-3247), PSMA 1.8.3 (PTA-3906), PSMA A3.1.3 (PTA-3904), PSMA A3.3.1 (PTA-3905), Abgenix 4.248.2 (PTA-4427), Abgenix 4.360.3 (PTA-4428), Abgenix 4.7.1 (PTA-4429), Abgenix 4.4.1 (PTA-4556), Abgenix 4.177.3 (PTA-4557), Abgenix 4.16.1 (PTA-4357), Abgenix 4.22.3 (PTA-4358), Abgenix 4.28.3 (PTA-4359), Abgenix 4.40.2 (PTA-4360), Abgenix 4.48.3 (PTA-4361), Abgenix 4.49.1 (PTA-4362), Abgenix 4.209.3 (PTA-4365), Abgenix 4.219.3 (PTA-4366), Abgenix 4.288.1 (PTA-4367), Abgenix 4.333.1 (PTA-4368), Abgenix 4.54.1 (PTA-4363), Abgenix 4.153.1 (PTA-4388), Abgenix 4.232.3 (PTA-4389), Abgenix 4.292.3 (PTA-4390), Abgenix 4.304.1 (PTA-4391), Abgenix 4.78.1 (PTA-4652), and Abgenix 4.152.1(PTA-4653).

In certain other embodiments, the antibody or antigen-binding fragment thereof binds to a conformational epitope and/or is internalized into a cell along with the prostate specific membrane antigen. In other embodiments, the isolated antibody or antigen-binding fragment thereof is bound to a label, preferably one selected from the group consisting of a fluorescent label, an enzyme label, a radioactive label, a nuclear magnetic resonance active label, a luminescent label, and a chromophore label.

In still other embodiments, the isolated antibody or antigen-binding fragment thereof is bound to at least one therapeutic moiety, such as a drug, preferably a cytotoxic drug, a replication-selective virus, a toxin or a fragment thereof, or an enzyme or a fragment thereof. Preferred cytotoxic drug include: calicheamicin, esperamicin, methotrexate, doxorubicin, melphalan, chlorambucil, ARA-C, vindesine, mitomycin C, cis-platinum, etoposide, bleomycin, 5-fluorouracil, estramustine, vincristine, etoposide, doxorubicin, paclitaxel, docetaxel, dolastatin 10, auristatin E and auristatin PHE. In other embodiments, the therapeutic moiety is an immunostimulatory or immunomodulating agent, preferably one selected from the group consisting of: a cytokine, chemokine and adjuvant.

In some embodiments, the antibodies or antigen-binding fragments of the invention specifically bind cell-surface PSMA and/or rsPSMA with a binding affinity of about 1×10⁻⁹M or less. Preferably, the binding affinity is about 1×10⁻¹⁰M or less, more preferably the binding affinity is about 1×10⁻¹¹M or less. In other embodiments the binding affinity is less than about 5×10⁻¹⁰M.

In additional embodiments, the antibodies or antigen-binding fragments of the invention mediate specific cell killing of PSMA-expressing cells with an IC₅₀s of less than about 1×10⁻¹⁰M. Preferably the IC₅₀s is less than about 1×10⁻¹¹M. More preferably the IC₅₀s is less than about 1×10⁻¹²M. In other embodiments the IC₅₀s is less than about 1.5×10⁻¹¹M.

In yet other embodiments, the isolated antibody or antigen-binding fragment thereof is bound to a radioisotope. The radioisotope can emit α radiations, β radiations, or γ radiations. Preferably the radioisotope is selected from the group consisting of ²²⁵Ac, ²¹¹ At, ²¹²Bi, ²¹³ Bi, ¹⁸⁶Rh, 188 ¹⁷⁷Lu, ⁹⁰Y, ¹³¹I, ⁶⁷Cu, ¹²⁵I, ¹²³I, ⁷⁷Br, ¹⁵³Sm, ¹⁶⁶Ho, ⁶⁴Cu, ²¹²Pb, ²²⁴Ra and ²²³Ra.

According to another aspect of the invention, hybridoma cell lines are provided that produce an antibody selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1 and Abgenix 4.152.1. In some embodiments, the hybridoma cell line is selected from the group consisting of PSMA 3.7 (PTA-3257), PSMA 3.8, PSMA 3.9 (PTA-3258), PSMA 3.11 (PTA-3269), PSMA 5.4 (PTA-3268), PSMA 7.1 (PTA-3292), PSMA 7.3 (PTA-3293), PSMA 10.3 (PTA-3247), PSMA 1.8.3 (PTA-3906), PSMA A3.1.3 (PTA-3904), PSMA A3.3.1 (PTA-3905), Abgenix 4.248.2 (PTA-4427), Abgenix 4.360.3 (PTA-4428), Abgenix 4.7.1 (PTA-4429), Abgenix 4.4.1 (PTA-4556), Abgenix 4.177.3 (PTA-4557), Abgenix 4.16.1 (PTA-4357), Abgenix 4.22.3 (PTA-4358), Abgenix 4.28.3 (PTA-4359), Abgenix 4.40.2 (PTA-4360), Abgenix 4.48.3 (PTA-4361), Abgenix 4.49.1 (PTA-4362), Abgenix 4.209.3 (PTA-4365), Abgenix 4.219.3 (PTA-4366), Abgenix 4.288.1 (PTA-4367), Abgenix 4.333.1 (PTA-4368), Abgenix 4.54.1 (PTA-4363), Abgenix 4.153.1 (PTA-4388), Abgenix 4.232.3 (PTA-4389), Abgenix 4.292.3 (PTA-4390), Abgenix 4.304.1 (PTA-4391), Abgenix 4.78.1 (PTA-4652), and Abgenix 4.152.1(PTA-4653).

According to a further aspect of the invention, compositions are provided that include the foregoing antibodies or antigen-binding fragments thereof and a pharmaceutically acceptable carrier, excipient, or stabilizer. Other compositions include a combination of two or more of the foregoing antibodies or antigen-binding fragments thereof and a pharmaceutically acceptable carrier, excipient, or stabilizer. In some embodiments, the compositions also include an antitumor agent, an immunostimulatory agent, an immunomodulator, or a combination thereof. Preferred antitumor agents include a cytotoxic agent, an agent that acts on tumor neovasculature, or a combination thereof. Preferred immunomodulators include α-interferon, γ-interferon, tumor necrosis factor-α or a combination thereof. Preferred immunostimulatory agents include interleukin-2, immunostimulatory oligonucleotides, or a combination thereof.

According to another aspect of the invention antibodies or antigen-binding fragments thereof that mediate antibody-dependent cellular cytotoxicity (ADCC) are provided. In some embodiments these antibodies or antigen-binding fragments thereof mediate ADCC of human prostate cancer cells. In other embodiments the antibodies are human antibodies. In still other embodiments the antibodies are capable of causing at least 5%, 10%, 15%, 20%, 25%, 30%, 35%, 40%, 50%, 75% or more cell lysis in vitro with an effector to target ratio of 5:1, 10:1, 15:1, 20:1, 25:1, 30:1, 40:1, 50:1 or more. In other embodiments these antibodies mediate more ADCC than control antibodies.

According to another aspect of the invention, kits for detecting prostate cancer for diagnosis, prognosis or monitoring are provided. The kits include the foregoing isolated labeled antibody or antigen-binding fragment thereof, and one or more compounds for detecting the label. Preferably the label is selected from the group consisting of a fluorescent label, an enzyme label, a radioactive label, a nuclear magnetic resonance active label, a luminescent label, and a chromophore label.

The invention in another aspect provides one or more of the foregoing isolated antibodies or antigen-binding fragments thereof packaged in lyophilized form, or packaged in an aqueous medium.

In another aspect of the invention, methods for detecting the presence of PSMA, or a cell expressing PSMA, in a sample are provided. The methods include contacting the sample with any of the foregoing antibodies or antigen-binding fragments thereof which specifically bind to an extracellular domain of PSMA, for a time sufficient to allow the formation of a complex between the antibody or antigen-binding fragment thereof and PSMA, and detecting the PSMA-antibody complex or PSMA-antigen-binding fragment complex. The presence of a complex in the sample is indicative of the presence in the sample of PSMA or a cell expressing PSMA.

In another aspect, the invention provides other methods for diagnosing a PSMA-mediated disease in a subject. The methods include administering to a subject suspected of having or previously diagnosed with PSMA-mediated disease an amount of any of the foregoing antibodies or antigen-binding fragments thereof which specifically bind to an extracellular domain of prostate specific membrane antigen. The method also includes allowing the formation of a complex between the antibody or antigen-binding fragment thereof and PSMA, and detecting the formation of the PSMA-antibody complex or PSMA-antigen-binding fragment antibody complex to the target epitope. The presence of a complex in the subject suspected of having or previously diagnosed with prostate cancer is indicative of the presence of a PSMA-mediated disease.

In certain embodiments of the methods, the PSMA-mediated disease is prostate cancer. In other embodiments, the PSMA-mediated disease is a non-prostate cancer, such as those selected from the group consisting of bladder cancer including transitional cell carcinoma; pancreatic cancer including pancreatic duct carcinoma; lung cancer including non-small cell lung carcinoma; kidney cancer including conventional renal cell carcinoma; sarcoma including soft tissue sarcoma; breast cancer including breast carcinoma; brain cancer including glioblastoma multiforme; neuroendocrine carcinoma; colon cancer including colonic carcinoma; testicular cancer including testicular embryonal carcinoma; and melanoma including malignant melanoma.

In preferred embodiments of the foregoing methods, the antibody or antigen-binding fragment thereof is labeled. In other embodiments of the foregoing methods, a second antibody is administered to detect the first antibody or antigen-binding fragment thereof.

In a further aspect of the invention, methods for assessing the prognosis of a subject with a PSMA-mediated disease are provided. The methods include administering to a subject suspected of having or previously diagnosed with PSMA-mediated disease an effective amount of an antibody or antigen-binding fragment thereof according to claim A1 or B1, allowing the formation of a complex between the antibody or antigen-binding fragment thereof and PSMA, and detecting the formation of the complex to the target epitope. The amount of the complex in the subject suspected of having or previously diagnosed with PSMA-mediated disease is indicative of the prognosis.

In another aspect of the invention, methods for assessing the effectiveness of a treatment of a subject with a PSMA-mediated disease are provided. The methods include administering to a subject suspected treated for a PSMA-mediated disease an effective amount of the foregoing antibodies or antigen-binding fragments thereof, allowing the formation of a complex between the antibody or antigen-binding fragment thereof and PSMA, and detecting the formation of the complex to the target epitope. The amount of the complex in the subject suspected of having or previously diagnosed with PSMA-mediated disease is indicative of the effectiveness of the treatment.

In certain embodiments of these two aspects of the invention, the PSMA-mediated disease is prostate cancer. In other embodiments, the PSMA-mediated disease is a non-prostate cancer. In those embodiments, the non-prostate cancer preferably is selected from the group consisting of bladder cancer including transitional cell carcinoma; pancreatic cancer including pancreatic duct carcinoma; lung cancer including non-small cell lung carcinoma; kidney cancer including conventional renal cell carcinoma; sarcoma including soft tissue sarcoma; breast cancer including breast carcinoma; brain cancer including glioblastoma multiforme; neuroendocrine carcinoma; colon cancer including colonic carcinoma; testicular cancer including testicular embryonal carcinoma; and melanoma including malignant melanoma. In still other embodiments, the antibody or antigen-binding fragment thereof is labeled. In further embodiments, a second antibody is administered to detect the first antibody or antigen-binding fragment thereof.

According to yet another aspect of the invention, methods for inhibiting the growth of a cell expressing PSMA are provided. The methods include contacting a cell expressing PSMA with an amount of at least one of the foregoing antibodies or antigen-binding fragments thereof which specifically binds to an extracellular domain of PSMA effective to inhibit the growth of the cell expressing PSMA.

According to another aspect of the invention, methods for inducing cytolysis of a cell expressing PSMA are provided. The methods include contacting a cell expressing PSMA with an amount of at least one of the foregoing antibodies or antigen-binding fragments thereof which specifically binds to an extracellular domain of PSMA effective to induce cytolysis of the cell expressing PSMA. In certain embodiments, the cytolysis occurs in the presence of an effector cell. In other embodiments, the cytolysis is complement mediated.

According to still another aspect of the invention, methods for treating or preventing a PSMA-mediated disease are provided. The methods include administering to a subject having a PSMA-mediated disease an effective amount of at least one of the forgoing antibodies or antigen-binding fragments thereof to treat or prevent the PSMA-mediated disease. In some embodiments, the PSMA-mediated disease is a cancer, such as prostate cancer or a non-prostate cancer (including the nonprostate cancers described elsewhere herein).

In yet a further aspect of the invention, methods for treating or preventing a PSMA-mediated disease are provided. The methods include administering to a subject having a PSMA-mediated disease or at risk of having a PSMA-mediated disease an amount of at least one of the foregoing antibodies or antigen-binding fragments thereof effective to treat or prevent the PSMA-mediated disease.

In some embodiments, the PSMA-mediated disease is a cancer, such as prostate cancer or a non-prostate cancer (including the nonprostate cancers described elsewhere herein).

In other embodiments, the method also includes administering another therapeutic agent to treat or prevent the PSMA-mediated disease at any time before, during or after the administration of the antibody or antigen-binding fragment thereof. In some of these embodiments, the therapeutic agent is a vaccine, and preferably the vaccine immunizes the subject against PSMA.

In still other embodiments, the antibody or antigen-binding fragment thereof is bound to at least one therapeutic moiety, preferably a cytotoxic drug, a drug which acts on the tumor neovasculature and combinations thereof. Preferred cytotoxic drugs are selected from the group consisting of: calicheamicin, esperamicin, methotrexate, doxorubicin, melphalan, chlorambucil, ARA-C, vindesine, mitomycin C, cis-platinum, etoposide, bleomycin, 5-fluorouracil, estramustine, vincristine, etoposide, doxorubicin, paclitaxel, docetaxel, dolastatin 10, auristatin E and auristatin PHE.

In other embodiments, the antibody or antigen-binding fragment thereof is bound to a radioisotope and the radiations emitted by the radioisotope is selected from the group consisting of α, β and γ radiations. Preferably, the radioisotope is selected from the group consisting of ²²⁵Ac, ²¹¹At, ²¹²Bi, ²¹³Bi, ¹⁸⁶Rh, ¹⁸⁸Rh, ¹⁷⁷Lu, ⁹⁰Y, ¹³¹I, ⁶⁷Cu, ¹²⁵I, ¹²³I, ⁷⁷Br, ¹⁵³Sm, ¹⁶⁶Ho, ⁶⁴Cu, ²¹²Pb, ²²⁴Ra and ²²³Ra.

The present invention provides methods for modulating at least one enzymatic activity of PSMA. As used in preferred embodiments of the methods, “modulating” an enzymatic activity of PSMA means enhancing or inhibiting the enzymatic activity. Thus in certain aspects of the invention, methods for inhibiting an enzymatic activity of PSMA are provided, and in other aspects of the invention, methods for enhancing an enzymatic activity of PSMA are provided. The terms “enhancing” and “inhibiting” in this context indicate that the enzymatic activity of PSMA is enhanced or inhibited in the presence of an antibody that specifically binds PSMA, or antigen-binding fragment thereof, relative to the level of activity in the absence of such an antibody or antigen-binding fragment thereof. Enzymatic activities of PSMA include folate hydrolase activity, N-acetylated α-linked acidic dipeptidase (NAALADase) activity, dipeptidyl dipeptidase IV activity and γ-glutamyl hydrolase activity.

Thus the invention in another aspect provides methods for modulating folate hydrolase activity. In certain embodiments of these methods, the activity is inhibited and in other embodiments, the activity is enhanced. The methods include contacting a folate hydrolase polypeptide with an amount of the foregoing isolated antibody or antigen-binding fragment thereof, under conditions wherein the isolated antibody or antigen-binding fragment thereof modulates the folate hydrolase activity. The folate hydrolase polypeptide can be isolated, contained in a sample such as a cell, a cell homogenate, a tissue, or a tissue homogenate, or contained in an organism. The organism preferably is an animal, particularly preferably a mammal.

In another aspect of the invention, methods for modulating N-acetylated α-linked acidic dipeptidase (NAALADase) activity are provided. In certain embodiments of these methods, the activity is inhibited and in other embodiments, the activity is enhanced. The methods include contacting a NAALADase polypeptide with an amount of the foregoing isolated antibody or antigen-binding fragment thereof under conditions wherein the isolated antibody or antigen-binding fragment thereof modulates NAALADase activity. The NAALADase polypeptide can be isolated, contained in a sample such as a cell, a cell homogenate, a tissue, or a tissue homogenate, or contained in an organism. The organism preferably is an animal, particularly preferably a mammal.

In yet another aspect of the invention, methods for modulating dipeptidyl dipeptidase IV activity are provided. In certain embodiments of these methods, the activity is inhibited and in other embodiments, the activity is enhanced. The methods include contacting a dipeptidyl dipeptidase IV polypeptide with an amount of the foregoing isolated antibody or antigen-binding fragment thereof under conditions wherein the isolated antibody or antigen-binding fragment thereof modulates dipeptidyl dipeptidase IV activity. The dipeptidyl dipeptidase IV polypeptide can be isolated, contained in a sample such as a cell, a cell homogenate, a tissue, or a tissue homogenate, or contained in an organism. The organism preferably is an animal, particularly preferably a mammal.

In yet another aspect of the invention, methods for modulating γ-glutamyl hydrolase activity are provided. In certain embodiments of these methods, the activity is inhibited and in other embodiments, the activity is enhanced. The methods include contacting a γ-glutamyl hydrolase polypeptide with an amount of the foregoing isolated antibody or antigen-binding fragment thereof under conditions wherein the isolated antibody or antigen-binding fragment thereof modulates γ-glutamyl hydrolase activity. The γ-glutamyl hydrolase polypeptide can be isolated, contained in a sample such as a cell, a cell homogenate, a tissue, or a tissue homogenate, or contained in an organism. The organism preferably is an animal, particularly preferably a mammal.

Methods of specific delivery of at least one therapeutic agent to PSMA-expressing cells are provided according to another aspect of the invention. The methods include administering an effective amount of at least one of the foregoing antibodies or antigen-binding fragments thereof conjugated to the at least one therapeutic agent. In some embodiments, the therapeutic agent is a nucleic acid molecule, an antitumor drug, a toxin or a fragment thereof, an enzyme or a fragment thereof, a replication-selective virus, or an immunostimulatory or immunomodulating agent. Preferred antitumor drugs include cytotoxic drugs, drugs which act on the tumor neovasculature and combinations thereof. Preferred cytotoxic drugs include calicheamicin, esperamicin, methotrexate, doxorubicin, melphalan, chlorambucil, ARA-C, vindesine, mitomycin C, cis-platinum, etoposide, bleomycin, 5-fluorouracil, estramustine, vincristine, etoposide, doxorubicin, paclitaxel, docetaxel, dolastatin 10, auristatin E and auristatin PHE. Preferred immunostimulatory or immunomodulating agent included cytokines, chemokines and adjuvants.

In still another aspect of the invention, isolated antibodies that selectively bind a PSMA protein multimer are provided. In preferred embodiments, the PSMA protein multimer is a dimer, and preferably at least one of the PSMA proteins forming the multimer is a recombinant, soluble PSMA (rsPSMA) polypeptide. Preferably the rsPSMA polypeptide consists essentially of amino acids 44-750 of SEQ ID NO: 1.

In a further aspect of the invention, isolated antibodies are provided that selectively bind a PSMA protein multimer and modulate one or more enzymatic activities of the PSMA protein multimer. As used in preferred embodiments of this aspect of the invention, “modulating” an enzymatic activity of a PSMA multimer means enhancing or inhibiting the enzymatic activity. Thus in certain aspects of the invention, antibodies that inhibit an enzymatic activity of PSMA multimers are provided, and in other aspects of the invention, antibodies that inhibit an enzymatic activity of PSMA multimers are provided. The terms “enhancing” and “inhibiting” in this context indicate that the enzymatic activity of a PSMA multimer is enhanced or inhibited in the presence of an antibody that specifically binds the PSMA multimers, or antigen-binding fragment thereof, relative to the level of activity in the absence of such an antibody or antigen-binding fragment thereof. In some embodiments, the enzymatic activity is selected from the group consisting of folate hydrolase activity, NAALADase activity, dipeptidyl dipeptidase IV activity and γ-glutamyl hydrolase activity. In other embodiments, the enzymatic activity is in the extracellular domain of the PSMA molecule. In still other embodiments, the antibody or antigen-binding fragment thereof specifically binds to an extracellular domain of PSMA.

In a further aspect, an isolated antibody or antigen-binding fragment thereof is provided that selectively binds a PSMA protein multimer. In this aspect, the isolated antibody is raised by immunizing an animal with a preparation comprising a PSMA protein multimer. Preferred preparations used in raising the antibody include those having at least about 10%, 20%, 30%, 40%, 50%, 75%, 90%, or 95% PSMA protein multimer. Preferably the PSMA protein multimer is a dimer.

In yet another aspect of the invention, compositions are provided that include one or more of the foregoing isolated antibodies, and an immunostimulatory molecule, such as an adjuvant and/or and a cytokine. Preferably the immunostimulatory molecule is IL-2 or an immunostimulatory oligonucleotide. In certain embodiments, the foregoing compositions also include a pharmaceutically-acceptable carrier.

The invention also includes methods for inducing an immune response, including administering to a subject in need of such treatment an effective amount of the foregoing isolated antibodies or compositions.

The invention provides, in another aspect, isolated antibodies or antigen-binding fragments thereof that selectively bind a PSMA protein multimer and modulate at least one enzymatic activity of PSMA. As used in preferred embodiments of this aspect of the invention, “modulating” an enzymatic activity of a PSMA means enhancing or inhibiting the enzymatic activity. Thus in certain aspects of the invention, antibodies that inhibit an enzymatic activity of PSMA are provided, and in other aspects of the invention, antibodies that inhibit an enzymatic activity of PSMA are provided. The terms “enhancing” and “inhibiting” in this context indicate that the enzymatic activity of PSMA is enhanced or inhibited in the presence of an antibody that specifically binds PSMA, or antigen-binding fragment thereof, relative to the level of activity in the absence of such an antibody or antigen-binding fragment thereof. The enzyme, in certain embodiments, is selected from the group consisting of hydrolases and peptidases. Preferred hydrolases include folate hydrolase and γ-glutamyl hydrolase. In a particularly preferred embodiment of PSMA inhibition, the hydrolase is folate hydrolase and the antibody is mAb 5.4 or mAb 3.9. Preferred peptidases include NAALADase and dipeptidyl dipeptidase IV. In some embodiments, the enzyme is active in cancer cells and has lesser activity in normal cells than in cancer cells or, preferably, no activity in normal cells. In preferred embodiments, the cancer cells in which the enzyme is active are prostate cancer cells. Compositions including the foregoing isolated antibodies or antigen-binding fragments thereof, and a pharmaceutically acceptable carrier, also are provided by the invention.

In another aspect of the invention, compositions are provided that include an isolated PSMA protein multimer. Preferably the PSMA protein multimer is a dimer. In certain embodiments, the compositions include at least about 10%, 20%, 30%, 40%, 50%, 75%, 90%, or 95% PSMA protein multimer. In other embodiments, the PSMA protein multimer comprises noncovalently associated PSMA proteins. The PSMA proteins preferably are noncovalently associated under nondenaturing conditions.

In certain embodiments of the foregoing compositions, at least one of the PSMA proteins forming the multimer is a recombinant, soluble PSMA (rsPSMA) polypeptide. In other embodiments, the PSMA protein multimer is reactive with a conformation-specific antibody that specifically recognizes PSMA. Preferably, the PSMA protein multimer comprises PSMA proteins in a native conformation and/or the PSMA multimer is enzymatically active. In preferred embodiments, the enzymatic activity is folate hydrolase activity, NAALADase activity, dipeptidyl dipeptidase IV activity and/or γ-glutamyl hydrolase activity.

In still other embodiments, the foregoing compositions also include an adjuvant and/or a cytokine or other immunostimulatory molecule. Preferred cytokines include IL-2, IL-12, IL-18 and GM-CSF. In further embodiments, the foregoing compositions also include a pharmaceutically acceptable carrier.

According to yet another aspect of the invention, methods for inducing an immune response are provided. The methods include administering to a subject in need of such treatment an effective amount of one or more of the foregoing compositions.

In a further aspect, the invention includes isolated recombinant soluble PSMA (rsPSMA) protein multimers, and isolated rsPSMA protein dimers. In some embodiments, the dimer includes noncovalently associated rsPSMA proteins, and preferably the rsPSMA proteins are noncovalently associated under nondenaturing conditions. In other embodiments, the isolated rsPSMA dimer is reactive with a conformation-specific antibody that specifically recognizes PSMA.

In a certain preferred embodiment, the isolated rsPSMA dimer is enzymatically active, with the enzymatic activity selected from the group consisting of folate hydrolase activity, NAALADase activity, dipeptidyl dipeptidase IV activity and γ-glutamyl hydrolase activity.

In still another aspect of the invention, methods of screening for a candidate agent that modulates at least one enzymatic activity of a PSMA enzyme are provided. As used in preferred embodiments of the methods, “modulating” an enzymatic activity of PSMA means enhancing or inhibiting the enzymatic activity. Thus in certain aspects of the invention, methods for screening for a candidate agent that inhibits an enzymatic activity of PSMA are provided, and in other aspects of the invention, methods for screening for a candidate agent that enhances an enzymatic activity of PSMA are provided. The terms “enhancing” and “inhibiting” in this context indicate that the enzymatic activity of PSMA is enhanced or inhibited in the presence of a candidate agent relative to the level of activity in the absence of such an agent. The methods include mixing the candidate agent with an isolated PSMA protein multimer to form a reaction mixture, followed by adding a substrate for the PSMA enzyme to the reaction mixture, and determining the amount of a product formed from the substrate by the PSMA enzyme. A change in the amount of product formed in comparison to a control is indicative of an agent capable of modulating at least one enzymatic activity of the PSMA enzyme. A decrease in the amount of product formed in comparison to a control is indicative of an agent capable of inhibiting at least one enzymatic activity of the PSMA enzyme. An increase in the amount of product formed in comparison to a control is indicative of an agent capable of enhancing at least one enzymatic activity of the PSMA enzyme. In some embodiments the PSMA enzyme is selected from the group consisting of NAALADase, folate hydrolase, dipeptidyl dipeptidase IV and γ-glutamyl hydrolase. In other embodiments the PSMA multimer comprises recombinant, soluble PSMA. In yet other embodiments the candidate agent is selected from the group consisting of an antibody, a small organic compound, or a peptide.

In another aspect of the invention, candidate agents that modulate at least one enzymatic activity of PSMA are provided. The candidate agents are identified according to the foregoing methods. Thus in certain aspects of the invention, candidate agents that inhibit an enzymatic activity of PSMA are provided, and in other aspects of the invention, candidate agents that enhance an enzymatic activity of PSMA are provided. In certain embodiments, the agent is selected from a combinatorial antibody library, a combinatorial protein library, or a small organic molecule library.

The invention also provides methods for identifying compounds that promote dissociation of PSMA dimers. The methods include contacting a PSMA dimer with a compound under conditions that do not promote dissociation of the PSMA dimer in the absence of the compound, measuring the amount of PSMA monomer and/or dimer; and comparing the amount of PSMA monomer and/or dimer measured in the presence of the compound with that observed in the absence of the compound. An increase in the amount of PSMA monomer measured in the presence of the compound indicates that the compound is capable of promoting dissociation of the PSMA dimer. A decrease in the amount of PSMA dimer measured in the presence of the compound indicates that the compound is capable of promoting dissociation of the PSMA dimer. When the amounts of PSMA monomer and PSMA dimer are measured, the methods can include calculating a ratio of PSMA monomer to PSMA dimer and comparing the ratio obtained in the presence of the compound with that obtained in the absence of the compound. In such methods, an increase in the ratio measured in the presence of the compound indicates that the compound is capable of promoting dissociation of the PSMA dimer.

The use of the foregoing compositions, molecules and agents in the preparation of medicaments also is provided. In preferred embodiments, the medicaments are useful in the treatment of conditions related to hyperproliferative diseases including cancer, and diseases of inappropriate NAALADase activity, folate hydrolase activity, dipeptidyl dipeptidase IV activity and/or γ-glutamyl hydrolase activity.

These and other aspects of the invention will be described in further detail in connection with the detailed description of the invention.

### BRIEF DESCRIPTION OF THE DRAWINGS

FIG. 1 depicts PSMA reactivity of mAbs as determined by flow cytometry. Anti-PSMA mAbs (3.7, 3.9, 3.11, 3.12, 5.4, and 10.3) incubated with either parental 3T3 cells (denoted by black lines) or 3T3 cells engineered to express cell-surface PSMA (3T3-PSMA; gray lines).

FIG. 2 shows a digitized image of immunoprecipitation of PSMA by mAbs. Lysates from 3T3-PSMA cells or parental 3T3 cells were incubated with each mAb and then precipitated using Protein A/G agarose beads. After washing, proteins were resolved on a polyacrylamide gel, blotted onto nitrocellulose membranes and visualized using the MAB544 anti-PSMA mAb.

FIG. 3 shows the recognition of non-denatured PSMA by several PSMA antibodies that recognize PSMA conformation.

FIG. 4 is a digitized image of a Western blot that shows the recognition of denatured PSMA by two PSMA antibodies and shows that antibodies that recognize PSMA conformation do not recognize denatured PSMA.

FIG. 5 is a digitized image of a polyacrylamide gel that shows an analysis of purified recombinant, soluble PSMA (rsPSMA) and of full-length PSMA from 3T3 cells (3T3 PSMA) or LNCaP cells (LNCaP PSMA) by reduced and non-reduced SDS-PAGE.

FIG. 6 provides the results for the determination of the dimeric structure of PSMA. FIG. 6A is a digitized image of a polyacrylamide gel that depicts a Blue Native PAGE analysis of purified recombinant, soluble PSMA (Purified rsPSMA) and of full-length PSMA extracted from 3T3 cells (3T3 PSMA) or LNCaP cells (LNCaP PSMA). FIG. 6B shows the results of the analytical size exclusion chromatography (SEC) of purified rsPSMA in neutral PBS buffer. The arrows indicate the retention times of protein standards. The retention time of 260 kDa for rsPSMA is consistent with that of a homodimer.

FIG. 7 illustrates that the dimeric but not monomeric rsPSMA (also referred to as PSMA_{ECTO}) is enzymatically active. Dimeric and monomeric PSMA were tested for folate hydrolase activity (FIG. 7A) and NAALADase activity (FIG. 7B). The background activity observed for PSMA monomer is consistent with residual amount (approximately 4%) of dimer present in the preparation.

FIG. 8 shows the effect of four antibodies (mAb 3.9, mAb 5.4, mAb 7.3 and mAb J591) on the enzymatic activity of folate hydrolase through measuring the rate of cleavage of glutamate from methotrexate di-gamma glutamate by folate hydrolase present in 0.0002 μg rsPSMA #7.

FIG. 9 shows the effect of four antibodies (mAb 3.9, mAb 5.4, mAb 7.3 and mAb J591) on the enzymatic activity of folate hydrolase through measuring the rate of cleavage of glutamate from methotrexate di-gamma glutamate by folate hydrolase present in 0.0002 μg rsPSMA #8.

FIG. 10 shows the effect of four antibodies (mAb 3.9, mAb 5.4, mAb 7.3 and mAb J591) on the enzymatic activity of folate hydrolase through measuring the rate of cleavage of glutamate from methotrexate di-gamma glutamate by folate hydrolase present in lysates of C4-2 cells.

FIG. 11 shows the impact of four antibodies (human mAbs 006, 026 and 4.40.2 as well as murine mAb 5.4) on PSMA folate hydrolase activity.

FIG. 12 illustrates the rapid and efficient internalization of ¹¹¹In labeled mAb 026 incubated with C4-2 cells.

FIG. 13 depicts the cloning protocol for IgG1 antibody cloning into pcDNA.

FIG. 14 provides the plasmid map of a nucleic acid molecule encoding the heavy chain of antibody AB-PG1-XG1-006.

FIG. 15 provides the plasmid map of a nucleic acid molecule encoding the heavy chain of antibody AB-PG1-XG1-026.

FIG. 16 provides the plasmid map of a nucleic acid molecule encoding the heavy chain of antibody AB-PG1-XG1-051.

FIG. 17 provides the plasmid map of a nucleic acid molecule encoding the heavy chain of antibody AB-PG1-XG1-069.

FIG. 18 provides the plasmid map of a nucleic acid molecule encoding the heavy chain of antibody AB-PG1-XG1-077.

FIG. 19 provides the plasmid map of a nucleic acid molecule encoding the heavy chain of antibody PSMA 10.3.

FIG. 20 provides the plasmid map of a nucleic acid molecule encoding the light chain of antibody AB-PG1-XG1-006.

FIG. 21 provides the plasmid map of a nucleic acid molecule encoding the light chain of antibody AB-PG1-XG1-026.

FIG. 22 provides the plasmid map of a nucleic acid molecule encoding the light chain of antibody AB-PG1-XG1-051.

FIG. 23 provides the plasmid map of a nucleic acid molecule encoding the light chain of antibody AB-PG1-XG1-069.

FIG. 24 provides the plasmid map of a nucleic acid molecule encoding the light chain of antibody AB-PG1-XG1-077.

FIG. 25 provides the plasmid map of a nucleic acid molecule encoding the light chain of antibody PSMA 10.3.

FIG. 26 depicts the cytotoxicity of ²²⁵Ac-3.9 on LNCaP target cells.

FIG. 27 illustrates the reactivity of anti-PSMA monoclonal antibodies XG-006, XG-051, 4.40.1, 4.49.1, 4.292.1 and 4.304.1 incubated with either parent 3T3 cells (black histogram) or 3T3 cells engineered to express cell-surface human PSMA (red histogram) and analyzed by flow cytometry.

FIG. 28 illustrates the binding of the anti-PSMA Abs. FIG. 28A shows that anti-PSMA mAbs bind to 3T3-PSMA cells and not 3T3 cells. One representative experiment from at least ten determinations is shown. FIG. 28B illustrates that binding to cell-surface PSMA using serial dilutions of anti-PSMA mAb-containing culture supernatants occurred. One representative experiment from five is shown. FIG. 28C shows binding to cell-surface PSMA using serial dilutions of purified anti-PSMA mAbs, XG-006 and 10.3 One representative experiment is shown.

FIG. 29 illustrates the immunotoxin cytotoxicity of murine anti-PSMA antibodies on C4-2 prostate cancer cells. SJ25C-1 as a control antibody is a murine anti-CD19 IgG. The LD 50 s (M) for 5.4, 3.9, and mJ591 antibodies were 2.27×10⁻¹¹, 2.29×10⁻¹¹ and 8.82×10⁻¹¹, respectively.

FIG. 30 illustrates the immunotoxin cytotoxicity of murine anti-PSMA antibodies on PSMA-3T3 cells. SJ25C-1 as a control antibody is a murine anti-CD19 IgG. The LD 50s (M) for 5.4, 3.9, and mJ591 antibodies were 1.64×10⁻¹¹, 1.96×10⁻¹¹ and 8.90×10⁻¹¹, respectively.

FIG. 31 provides the cytotoxicity of direct conjugated human 4.304 anti-PSMA antibodies with saporin on PSMA-3T3. The LD50 was 1.48×10⁻¹¹ M for direct conjugated 4.304 anti-PSMA antibodies with saporin.

FIG. 32 illustrates the results of the competition assay of unmodified 4.304, 4.40, mJ591 anti-PSMA antibodies used to compete with In-111 radiolabeled 4.40 and 4.304 anti-PSMA antibodies.

FIG. 33 illustrates the results of the competition assay of unmodified 4.304, mJ591 anti-PSMA antibodies used to compete with In-111 radiolabeled mJ591 anti-PSMA antibodies.

FIG. 34 shows an analysis of antibody PRGX1-XG-006 in association phase and dissociation phase at different concentrations of rsPSMA from 100 nM to 6.25 nM.

FIG. 35 shows the results of the comparison of the fully human anti-PSMA antibodies 4.40.1, 4.49.1, 051 and 006 and the murine anti-PSMA antibody 3.9 performed using Biacore analysis.

FIG. 36 provides results from the Scatchard analysis using In-111 labeled anti-PSMA antibody 3.9 of the PSMA-3T3, LNCaP and C4-2 cell lines.

FIG. 37 shows in vitro cytotoxicity of Ac-225 labeled human anti-PSMA antibody 4.40 on prostate cancer cells.

FIG. 38 shows the specific killing of PSMA expressing cells (C4-2) vs. PSMA non-expressing cells (PC-3) treated with ²²⁵Ac labeled mAb 026.

FIG. 39 shows the in vitro cytotoxicity of ²²⁵Ac labeled mAb 026 on human prostate cancer cell lines (C4-2 and LNCaP).

FIG. 40 shows the in vitro cytotoxicity of ²²⁵Ac labeled mAb 026 on human prostate cancer cell line, C4-2, evaluated by ³H thymidine incorporation.

FIG. 41 shows the results of in vivo radioimmunotherapy with Lu-177 labeled human anti-PSMA antibodies.

FIG. 42 provides the radio-HPLC profile and cell-based immunoreactivity of ¹⁷⁷Lu labeled antibodies (006, 026, mJ591 and HuIgG (control)).

FIG. 43 shows the specific binding of ¹⁷⁷Lu labeled antibodies (006, 026, mJ591 and IgG (control)) to PSMA positive tumors in vivo.

FIG. 44 shows the preferential retention of radiolabeled antibodies (006, 026, mJ591 and HuIgG) in PSMA+ tumors vs. PSMA− tumors as assessed by the percent activity in the tumors.

FIG. 45 provides data for normal organ (blood, liver, spleen, lungs, bone, heart and muscle) uptake (injected dose per gram of tissue, % ID/g) for the antibodies (006, 026, mJ591 and HuIgG).

FIG. 46 illustrates the therapeutic efficacy of ¹⁷⁷Lu labeled mAb 026 in PSMA-3T3 and 3T3 tumor-bearing mice.

FIG. 47 shows the preferential binding of mAb 006 to rsPSMA dimer.

FIG. 48 shows the preferential binding of mAb 026 to rsPSMA dimer.

FIG. 49 shows the binding of mAb 4.40 to rsPSMA dimer and monomer.

FIG. 50 shows the binding of mAb mJ591 to rsPSMA dimer and monomer.

FIG. 51 is a series of graphs that show flow cytometry data for the binding of anti-PSMA antisera to PSMA-3T3 cells. Antisera from mice immunized with a rsPSMA dimer preparation (ABIM151, ABIM152, ABIM153, ABIM154 and ABIM155) exhibited strong binding to PSMA-expressing cells. Antisera from mice immunized with a rsPSMA monomer preparation (ABIM156, ABIM157, ABIM158, ABIM159 and ABIM160) exhibited little or no binding to PSMA-expressing cells.

FIG. 52 provides the results showing antibody dependent cell-mediated cytotoxicity (ADCC) of human prostate cancer cells mediated by mAbs 006 and 026.

FIG. 53 shows the results of PSMA monomer-dimer equilibrium analysis. Purified dimeric (FIG. 53A) and monomeric (FIG. 53B) rsPSMA were subjected to various buffer conditions and analyzed for size by analytical size exclusion chromatography (SEC). The percentages of monomer (M) and dimer (D) are indicated. The monomer and dimer were initially contained in PBS+ buffer at a concentration of 0.2 mg/ml. The buffer conditions were adjusted as indicated, and the proteins were incubated at ambient temperature for the indicated time periods before SEC analysis.

### DETAILED DESCRIPTION OF THE INVENTION

The present invention provides, in part, multimeric, particularly dimeric, forms of PSMA protein, compositions and kits containing dimeric PSMA protein as well as methods of producing, purifying, processing and using these compositions. Such methods include methods for eliciting or enhancing an immune response to PSMA and/or cells expressing PSMA. Such methods include methods of producing antibodies to dimeric PSMA as well as methods of treating cancer, such as prostate cancer.

Prostate specific membrane antigen (PSMA) is a 100 kD Type II membrane glycoprotein expressed in prostate tissues and was originally identified by reactivity with a monoclonal antibody designated 7E11-C5 (Horoszewicz et al., 1987, Anticancer Res. 7:927-935; U.S. Pat. No. 5,162,504). PSMA was obtained in purified form (Wright et al., 1990, Antibody Immunoconjugates and Radio Pharmaceuticals 3:Abstract 193) and characterized as a type II transmembrane protein having sequence identity with the transferrin receptor (Israeli et al., 1994, Cancer Res. 54:1807-1811) and with NAALADase activity (Carter et al., 1996, Proc. Natl. Acad. Sci. U.S.A. 93:749-753). More importantly, PSMA is expressed in increased amounts in prostate cancer, and elevated levels of PSMA are also detectable in the sera of these patients (Horoszewicz et al., 1987; Rochon et al., 1994, Prostate 25:219-223; Murphy et al., 1995, Prostate 26:164-168; and Murphy et al., 1995, Anticancer Res. 15:1473-1479). PSMA expression increases with disease progression, becoming highest in metastatic, hormone-refractory disease for which there is no present therapy. Provocative recent data indicates that PSMA is also abundantly expressed on the neovasculature of a variety of other important tumors, including bladder, pancreas, sarcoma, melanoma, lung, and kidney tumor cells, but not on normal vasculature.

It has been discovered that PSMA in its native form is a homodimer. When ordinary isolation techniques are followed, however, the native form of PSMA is not typically maintained. Compositions of isolated PSMA protein that include isolated multimeric PSMA, particularly dimeric PSMA, therefore, are provided. These compositions include isolated PSMA protein, wherein at least about 5% of the isolated PSMA protein is in multimeric form. Other compositions are provided where at least about 10%, 15%, 20%, 25%, 30%, 35%, 40%, 45%, 50%, 55%, 60%, 65%, 70%, 75%, 80%, 85%, 90%, 95% or more of the isolated PSMA protein is in multimeric form. It has further been discovered that certain agents preserve or promote the multimeric, particularly the dimeric, association of isolated PSMA. Compositions of isolated PSMA protein that include these agents as well as methods of purifying and processing isolated PSMA protein compositions are, therefore, also provided.

As used herein “PSMA protein” includes the full-length PSMA protein (provided as SEQ ID NO: 1) or a portion thereof. These proteins are capable of forming multimers or aggregates of PSMA protein. As used herein, a “multimer or aggregate of PSMA protein” refers to the association of two or more PSMA proteins. Preferably, the PSMA proteins described herein are those that are capable of forming a dimer like that of native PSMA by non-covalent interactions or engineered to form a stable native-like dimer through covalent bonds, such as disulfide bonds. “A dimer like that of native PSMA” includes two PSMA proteins that are associated in the same way as the protein as found in nature or in such a way as to allow for the generation of antibodies that recognize at least one antigenic epitope of the native dimer (i.e., associate in a way such as to form an antigenic region as found in the native PSMA dimer or one capable of generating cross-reacting antibodies). The antibodies generated to the dimers provided herein are, therefore, capable of recognizing the native dimer. Preferably, the antibodies generated recognize native PSMA dimer but not PSMA monomer or have greater specificity for the native PSMA dimer than the monomer. In some embodiments, the PSMA proteins provided herein are larger aggregates of PSMA (i.e., three or more PSMA protein that are associated). These aggregates are likewise capable of generating antibodies that recognize PSMA. In some embodiments, these antibodies do not recognize PSMA monomer but do recognize native PSMA dimer. In other embodiments, these antibodies have greater specificity for the native PSMA dimer rather than PSMA monomer.

PSMA multimers are typically homomultimers (i.e., the associated PSMA proteins are the same). However, in some embodiments the PSMA multimers can be heteromultimers, particularly heterodimers. As used herein a “PSMA heteromultimer” is a multimer of PSMA proteins that is composed of at least two different PSMA proteins. Examples include two PSMA fragments, where one is slightly longer than the other or when one has a conservative amino acid substitution and the other does not. The heteromultimers provided herein, like homomultimers, are capable of generating antibodies that recognize native PSMA dimer. In preferred embodiments the antibodies raised against the PSMA heteromultimers recognize native PSMA dimer but not PSMA monomer. In still other preferred embodiments these antibodies have greater specificity for native PSMA dimer rather than PSMA monomer.

PMSA protein capable of forming multimers, particularly dimers, include the full-length protein (SEQ ID NO: 1). In some embodiments the PSMA protein capable of forming a multimer is the extracellular portion of PSMA (amino acids 44-750 of SEQ ID NO: 1). In other embodiments the PSMA protein capable of forming a multimer is PSM′ (amino acids 58-750 of SEQ ID NO: 1), an alternatively spliced form of PSMA. In yet other embodiments fragments of the full-length protein, the extracellular portion or PSM′ are capable of forming multimers. For example, these fragments include truncated PSMA proteins that begin at amino acid 44, 45, 46, 47, 48, 49, 50, 51, 52, 53, 54, 55, 56, 57, 58, 59, 60, 61, 62, 63, 64, 65, etc. of SEQ ID NO: 1 and end at amino acid 750 of SEQ ID NO: 1. Other such truncated proteins begin at amino acid 44 of SEQ ID NO: 1 and end at amino acid 749, 748, 747, 746, 745, 744, 743, 742, 741, 740, etc. of SEQ ID NO: 1. Still other truncated proteins include those that begin at amino acid 44, 45, 46, 47, 48, 49, 50, 51, 52, 53, 54, 55, 56, 57, 58, 59, 60, 61, 62, 63, 64, 65, etc. of SEQ ID NO: 1 and end at amino acid 749, 748, 747, 746, 745, 744, 743, 742, 741, 740, etc. of SEQ ID NO: 1. In some embodiments the truncated PSMA protein includes the amino acids 601-750 of SEQ ID NO: 1 or a functional portion thereof capable of forming dimers. As provided herein, the PSMA proteins are intended to encompass any fragment of the PSMA protein that is capable of forming a multimer as provided herein. Therefore, any portion of SEQ ID NO: 1 is included in this definition as well as its functional variant. Functional variants are described further herein below.

In some embodiments the isolated PSMA protein is not full-length PSM′ (amino acids 58-750 of SEQ ID NO: 1) or the full-length extracellular portion of PSMA (amino acids 44-750 of SEQ ID NO: 1). In other instances, the isolated PSMA protein is not full-length PSMA (SEQ ID NO: 1). The fragment can have a size of at least about 25, 50, 100, 125, 150, 175, 200, 250, 300, 350, 400, 450, 500, 550, 600, 650, 700, or 749 amino acids and every integer length therebetween. In some embodiments, these fragments include amino acids 63-68, 132-137 or 482-487 of SEQ ID NO:1. In some other preferred instances, the PSMA protein is not membrane-bound.

Compositions of PSMA protein with agents and/or solutions that preserve or promote the multimeric association, particularly the dimeric association, of PSMA also are provided. In some instances the agents are in a solution along with the PSMA protein but are not necessarily so. An agent or solution that “preserves or promotes the dimeric assocation of PSMA” is one that either maintains the dimeric association (dimerization) of PSMA over time or facilitates the the dimeric association of monomeric forms of the PSMA protein. For example, any solution that increases the amount of PSMA dimers, maintains the amount of PSMA dimers or retards the disassociation of PSMA dimers is encompassed by the above definition. Although the dimeric state is specifically recited, these terms are also intended to encompass other multimeric states of PSMA, and therefore, compositions, kits and methods of production and use of other multimers of PSMA.

Preferably, the “preservation or promotion of dimeric PSMA” refers to the maintenance of the dimeric state of PSMA protein for at least about 5%, 10%, 20%, 30%, 40%, 50%, 60%, 70%, 80%, 90%, 95% or more of the PSMA dimers initially present in a composition. Preferred compositions comprising the dimeric form of PSMA have less than about 35% of the monomeric form of PSMA, preferably less than about 20%, more preferably less than about 15% of the monomeric form. In one embodiment the composition has less than about 5% of the monomeric PSMA protein. The preservation or promotion of dimeric PSMA also refers to the conversion of about 5%, 10%, 20%, 30%, 40%, 50%, 60%, 70%, 80%, 90%, 95% or more of the initially monomeric PSMA to dimeric form.

The promotion of dimerization or maintenance of the dimeric state can occur at any of a number of experimental or storage temperatures. In some instances the promotion or preservation of dimeric PSMA occurs at a temperature of about 45° C. or lower. In other instances the promotion or preservation of dimeric PSMA is at a temperature of about 37° C. The promotion or preservation can also be at a temperature range of about 20° C. to about 30° C. or about or below room temperature. In other instances the promotion or preservation is at a range of about 4° C. to about 20° C. In still other instances the promotion or preservation is at about −20° C. to about 4° C. or about −80° C. to about −20° C. The promotion or preservation of the dimeric state of PSMA can also occur in a composition of PSMA protein that is in solution or in a freeze-dried form, e.g., lyophilized form. The dimeric state can also be promoted or preserved over any period of time. In some instances the period of time is at least about 1, 2, 3, 4, 5, 6, 10, 15, 20, 24, 48, 72 or more hours. In other instances the period of time is at least about 1, 2, 3, 4, 5, 10, 15, 20, 25, 30 or more days. In still other instances the period of time is at least about 1, 2, 3, 4, 5, 6, 7, 8, 9, 10, 12, 15, 20, 25, 30 or more weeks. In yet other instances, the period of time is at least about 1, 2, 3, 6, 9, 12 or more months or as long as 2 years or more. The formulations provided herein are stable during long-term storage, i.e., the formulations preserve or promote the dimeric PSMA state.

It was surprisingly discovered that pH alone can influence the dimeric state of PSMA. As described below in the Examples section, the pH at which a PSMA solution is incubated can influence the multimeric form of PSMA as well as its recovery. Incubation at various pHs for 4 days at a temperature of about 45° C. influenced the dimerization or aggregation of PSMA protein as well as the recovery of PSMA protein by analytical TSK gel filtration chromatography. The benefits of pH on the preservation of dimeric rsPSMA (2 mg/ml in PBS+) are retained when the protein solution is diluted 10-fold in a variety of buffer solutions, each containing 2 mM glycine, 2 mM citric acid, 2 mM Hepes, 2 mM MES and 2 mM Tris Base.

The dimeric structure of PSMA according to the invention is preserved at a pH in the range of about 4 to about 8. Therefore, a solution that preserves or promotes the dimerization of PSMA is one with a pH of about 4, 4.5, 5, 5.5, 6, 6.5, 7, 7.5, or 8. Recovery of dimeric PSMA from a column was better at a pH in the range of about 5 to about 7, and these pH values are preferred. In some instances, a pH of about 6 is preferred. Thus, the invention provides formulations of PSMA in solution, wherein the pH is in the range from about 4 to about 8, preferably in the range from about 5 to about 7, more preferably in the range from about 5.5 to about 7, and most preferably in the range from about 6 to about 7.

An “agent that preserves or promotes the dimeric association of PSMA” is meant to encompass an agent that promotes or maintains the dimerization of PSMA. Such agents have been found to include pH adjusting agents (as discussed above), metal ions and salts. It has been discovered that these agents, individually or in combination, are able to preserve or promote dimeric association of PSMA. In some embodiments it is the combination of the metal ion, salt or pH adjusting agent that can promote or preserve dimeric association of PSMA, while the individual metal ion, salt or pH adjusting agent cannot. As provided in the Examples, the use of chelating agents, such as EDTA, converted dimeric PSMA into the monomer. This result indicated that the presence of metal ions can positively affect the stability of the dimer. Additionally, PSMA shares modest sequence and structural homology with human transferrin receptor (TfR), which contains additional metal-binding sites within its helical domains (Lawrence, C. M., et al. (1999) Science 286, 779-782). Therefore, metal ions are considered to be agents which promote or preserve the dimeric state of PSMA protein. Such metals ions include, but are not limited to, zinc ions (e.g., Zn²⁺), calcium ions (e.g., Ca²⁺), magnesium ions (e.g., Mg²⁺). cobalt ions (e.g., Co²⁺), manganese ions (e.g., Mn²⁺) or combinations thereof.

In some instances these metal ions can be added to a composition of PSMA protein in the form of a salt. Such salts include zinc chloride, calcium chloride, magnesium chloride, cobalt chloride or manganese chloride. It has been further determined that compositions of PSMA protein, wherein the dimeric state is promoted or preserved, include at least about 0.1, 0.2, 0.3, 0.4, 0.5, 0.6, 0.7, 0.8, 0.9, 1, 2, 3, 4, 5 or more molar equivalents of metal ion to PSMA protein (total PSMA protein, i.e., total amount of PSMA protein molecules). In some instances, the molar equivalent of metal ions should be in molar excess to PSMA protein. In some specific solutions of PSMA protein (2 mg/ml in PBS+; diluted 10-fold), as provided in the Examples, it has further been found, that the metal ions are preferably present at a concentration in the range of about 0.1 mM to about 5 mM. The metal ions in some instances are present at a concentration in the range of about 0.1 mM to about 1 mM. In other embodiments the metal ions are present at a concentration in the range of about 0.1 mM to about 0.5 mM. In solutions where there is a combination of one or more types of metal ions, the one or more metal ions can be at the same concentration or at a different concentration. For example, one such solution can contain a concentration of calcium ions of about 0.5 mM and a concentration of zinc ions of a concentration that is greater than 0.1 mM but less than 0.5 mM. Because of the importance of metal ions in the dimerization of PSMA in some compositions, in some instances, it is preferred that the compositions do not contain a chelating agent.

It has also been found that salts preserve or promote PSMA dimerization. As shown below in the Examples, a dimer preparation that contained approximately 5% monomer initially was converted to 100% dimer upon incubation for 72 hours at ambient temperature in PBS+ (phosphate-buffered saline containing 1 mM Ca²⁺ and 0.5 mM Mg²⁺, pH 7.2) supplemented with 2M sodium chloride. For a preparation that initially comprised >95% monomer, high salt similarly drove the equilibrium to mostly (81%) dimer within 72 hours.

Salts that preserve or promote PSMA dimerization can include those with a cationic component selected from the group consisting of sodium, potassium, ammonium, magnesium, calcium, zinc and combinations thereof, and those with an anionic component selected from the group consisting of chloride, sulfate, acetate and combinations thereof. In preferred embodiments the salt is sodium chloride, sodium sulfate, sodium acetate or ammonium sulfate. The salt can be present in a PSMA-containing composition at any concentration that preserves or promotes the dimerization of PSMA. In some instances the salt is present at a concentration in the range of about 50 mM to about 2M. The salt preferably is present at a concentration of about 100 mM to 300 mM. The salt more preferably is present at a concentration of about 150 mM.

In some cases where a high salt concentration is used to promote or preserve PSMA dimerization, the salt concentration can be diluted to within a physiologically acceptable range suitable for parenteral use prior to administration. As an example, the salt concentration can be diluted with an adjuvant or a diluent. Diluents and adjuvants are both well known in the art. An adjuvant is a substance which potentiates the immune response. Specific examples of adjuvants include monophosphoryl lipid A (MPL, SmithKline Beecham); saponins including QS21 (SmithKline Beecham); immunostimulatory oligonucleotides (e.g., CpG oligonucleotides described by Kreig et al., Nature 374:546-9, 1995); incomplete Freund's adjuvant; complete Freund's adjuvant; montanide; vitamin E and various water-in-oil emulsions prepared from biodegradable oils such as squalene and/or tocopherol, Quil A, MPL and cell wall skeleton from mycobacterium combinations such as ENHANZYN™ (Corixa Corporation, Hamilton, Mont.), CRL-1005, L-121, alpha-galactosylceramide (Fujii et al., J. Exp. Med., 2003, Jul. 21; 198(2): 267-79) and combinations thereof. A preferred adjuvant is alum. Other diluents include water suitable for injection, saline, PBS, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Therefore, in some aspects of the invention a preferred composition comprising isolated PSMA protein is a solution that promotes or preserves multimeric, particularly dimeric, association of PSMA protein comprising 5 to 20 mM sodium phosphate, sodium acetate or a combination thereof; 100 to 300 mM sodium chloride or sodium sulfate; and 0.1 to 2 mM of at least one metal ion. The metal ions can be chosen from zinc ions, calcium ions, magnesium ions, cobalt ions, manganese ions or a combination thereof. The pH of such a solution can also be adjusted to be in a range of about 4 to 8, preferable 5 to 7 and most preferable 6 to 6.5. Such a solution can also, optionally, include an adjuvant such as alum.

Agents that preserve or promote PSMA dimerization can be used in compositions of PSMA protein or methods of processing such compositions. Furthermore, a method for identifying such agents is provided herein. Such a method includes the following steps: determining the amount of form of PSMA in a sample prior to exposure to a candidate agent; exposing the sample to the candidate agent; determining the amount of the form of PSMA in the sample after the exposure; and comparing the amount of the form of PSMA in the sample prior to and after the exposure to the candidate agent. The form of PSMA can be a monomer or multimer, prederably the dimer. An agent which preserves and/or promotes dimer formation of PSMA protein is suitable for use in the compositions comprising PSMA protein dimers.

As described below the effect of buffering agent on the ability of PSMA to dimerize or maintain its dimerization was also tested. It was found that many can be used in a solution of PSMA without negatively impacting the dimeric state of PSMA. The sole exception for solutions of PSMA protein with 150 mM NaCl at a pH of 6 was citrate buffer. Interestingly, citrate buffer is known to function as a chelating agent. Therefore the formulations of PSMA described herein can include any buffer so long as the buffer is not one with a chelating effect that outweighs the preservation or promoting effect of the other properties of the formulation. Preferably optimal buffers include those with buffering capacity at a pH in the range of about 4 to about 8. More preferably, buffers are those with buffering capacity at a pH in the range of about 5 to about 7. Most preferably the buffers are those that have buffering capacity at a pH in the range of about 5.5 to about 7. Buffers in general are well known to those of ordinary skill in the art. Buffer systems include citrate buffers, acetate buffers, borate buffers, and phosphate buffers. Specific examples of buffers include citric acid, sodium citrate, sodium acetate, acetic acid, sodium phosphate and phosphoric acid, sodium ascorbate, tartartic acid, maleic acid, glycine, sodium lactate, lactic acid, ascorbic acid, imidazole, sodium bicarbonate and carbonic acid, sodium succinate and succinic acid, histidine, and sodium benzoate and benzoic acid. Buffers also include PBS and Hepes.

The effect of free amino acids on the dimeric state of rsPSMA (2 mg/ml in PBS+) dialyzed into 20 mM sodium acetate and 150 mM NaCl at a pH of about 6 was also tested. In general it was found that free amino acids did not have a strong negative effect on dimer association of PSMA and/or column recovery, with the exception of histidine, glutamic acid and aspartic acid used individually at the specific experimental conditions. Therefore, the formulations provided herein can also include a free amino acid or combination of free amino acids, provided that the free amino acid does not have a negative effect that outweighs the dimeric association promoting or preserving nature of the specific formulation. Such free amino acids can be naturally occurring, modified or non-naturally occurring free amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels). Modified or non-naturally occurring free amino acids also include but are not limited to 2-aminoadipic acid; 3-aminoadipic acid; beta-alanine, beta-aminopropionic acid; 2-aminobutyric acid; 4-aminobutyric acid, piperidinic acid; 6-aminocaproic acid; 2-aminoheptanoic acid; 2-aminoisobutyric acid; 3-aminoisobutyric acid; 2-aminopimelic acid; 2, 4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine, sarcosine; N-methylisoleucine; 6-N-methyllysine; N-methylvaline; norvaline; norleucine and ornithine. In particular, free amino acids that do not have a negative effect on dimeric association of PSMA and/or column recovery include those that are non-acidic. Examples of these non-acidic free amino acids include glycine, proline, isoleucine, leucine, alanine and arginine.

In addition to free amino acids, surfactants and other excipients were also found not to have a negative impact on the dimeric state of PSMA. Therefore, surfactants as well as other excipients can be included in the compositions provided herein. Examples of surfactants include those known in the art and described herein. For example, surfactants include Triton X-100, dodecylmaltoside, cholic acid and CHAPS.

Examples of excipients include binders, coatings, compression/encapsulation aids, disintegrants, creams and lotions, lubricants, materials for chewable tablets, parenterals, plasticizers, powder lubricants, soft gelatin capsules, spheres for coating, spheronization agents, suspending/gelling agents, sweeteners and wet granulation agents. Specific examples of such excipients include acetyltriethyl citrate (ATEC); acetyltri-n-butyl citrate (ATBC); aspartame; aspartame and lactose; alginates; calcium carbonate; carbopol; carrageenan; cellulose acetate phthalate-based coatings; cellulose-based coatings; cellulose and lactose combinations; colorants for film coating systems; croscarmellose sodium; crospovidone; dextrose; dibutyl sebacate; ethylcellulose-based coatings; fructose; gellan gum; glyceryl behenate; honey; lactose; anhydrous; lactose; monohydrate; lactose and aspartame; lactose and cellulose; lactose and microcrystalline cellulose; L-HPC (Low-substituted HydroxyPryopl Cellulose); magnesium stearate; maltodextrin; maltose DC; mannitol DC; methylcellulose-based coatings; microcrystalline cellulose; methacrylate-based coatings; microcrystalline cellulose and carrageenan; microcrystalline cellulose and guar gum; microcrystalline cellulose and lactose; microcrystalline cellulose and sodium carboxymethylcellulose; molasses DC; polyvinyl acetate phathalate (PVAP); povidone; shellac; sodium starch glycolate; sorbitol, crystalline; sorbitol, special solution; starch DC; sucrose DC; sugar spheres; triacetin; triethylcitrate and xanthan gum. Other excipients include antioxidants and cryoprotectants.

Antioxidants are substances capable of inhibiting oxidation by removing free radicals from solution. Antioxidants are well known to those of ordinary skill in the art and include materials such as ascorbic acid, ascorbic acid derivatives (e.g., ascorbylpalmitate, ascorbylstearate, sodium ascorbate, calcium ascorbate, etc.), butylated hydroxy anisole, butylated hydroxy toluene, alkylgallate, dithiothreitol (DTT), sodium meta-bisulfite, sodium bisulfite, sodium dithionite, sodium thioglycollic acid, sodium formaldehyde sulfoxylate, tocopherol and derivatives thereof (e.g., d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, and sodium sulfite. Such materials are typically added in ranges from about 0.01 to about 2%.

For a lyophilized product or a product stored in the cold, one or more cryoprotectants can be added. Typical cryoprotectants for proteins include but are not limited to: sugars such as sucrose, lactose, glucose, trehalose, maltose, and the like; polyols such as inositol, ethylene glycol, glycerol, sorbitol, xylitol, mannitol, 2-methyl-2,4-pentane-diol and the like; amino acids such as Na glutamate, proline, alpha-alanine, beta-alanine, glycine, lysine-HCl, 4-hydroxyproline; polymers such as polyethylene glycol, dextran, polyvinylpyrrolidone and the like; inorganics salts such as sodium sulfate, ammonium sulfate, potassium phosphate, magnesium sulfate, and sodium fluoride and the like; organics salts such as sodium acetate, sodium polyethylene, sodium caprylate, proprionate, lactate, succinate and the like; as well as agents such as trimethylamine N-oxide, sarcosine, betaine, gamma-aminobutyric acid, octapine, alanopine, strombine, dimethylsulfoxide, and ethanol.

The invention also involves methods for preparing or processing compositions of PSMA protein. Aqueous solutions of PSMA protein are included in these methods. Some of these methods include the step of adjusting the pH so that it is in the range of about 4 to about 8. In some methods the pH is adjusted to be in the range of about about 5 to 7, and more preferably the pH is adjusted to be in the range of about 5.5 to 7. Most preferably the pH is adjusted to be about 6. The compositions can also contain any one or combination of an isotonicity agent, a buffering agent, a surfactant, an antioxidant, a cryoprotectant or other excipients. Preferably the compositions do not include a chelating agent.

According to another aspect of the invention, a composition of PSMA protein is processed by contacting the composition of PSMA protein with an agent that promotes or preserves the dimeric association of PSMA such as pH adjusting agents, metal ions and/or salts as provided above. Compositions that include these agents can also include agents selected from an isotonicity agent, a buffering agent, a surfactant, an antioxidant, a cryoprotectant and other excipients, but preferably, not a chelating agent. Such methods can also include futher steps of contacting the composition of PSMA protein with other dimer promoting or preserving agents and/or pH adjusting steps when the PSMA protein is in a solution.

Additionally, in another aspect of the invention, a method of purifying PSMA protein is also provided. The methods of purifying PSMA include the use of any of the agents and/or solutions described herein that preserve or promote the multimeric, particularly dimeric, association of PSMA in conjunction with any of the separation techniques that are known to those in the art. Such separation techniques include chromatography (e.g., TSK gel filtration chromatography) and are described in more detail in the Examples below. For instance, a separation technique encompassed within this aspect of the invention can include the steps of loading a sample onto a column, eluting or washing the sample from the column and collecting the eluted fractions. Such steps can be repeated any of a number of times to produce the desired PSMA protein composition. These steps can, optionally, also include steps whereby the sample containing PSMA protein is dialyzed. Preferably, the sample containing PSMA protein is dialyzed into a solution that preserves or promotes the multimeric association of PSMA. In one embodiment, the solutions used in these methods contain a metal ion or a salt. In other embodiments, the solution is at a pH that preserves or promotes PSMA multimerization. The metal ion and salts, including concentration ranges, as well as pH ranges that can be used in these purification methods have been provided above. In some preferred embodiments, the pH of the solution can be at about 7 or 7.5. In other preferred embodiments, the metals are calcium ions, magnesium ions or combinations thereof. The calcium and magnesium ions are present, for instance, at a concentration of about 1 mM and of about 0.5 mM, respectively. In other preferred embodiments the salt is present at a concentration of about 2M.

The amount of dimeric PSMA in the compositions provided herein is effective to elicit or enhance an immune response to cells expressing PSMA. The compositions can, therefore, be used to immunize an animal for the purpose of raising antibodies to dimeric PSMA. The compositions provided herein can also be used to treat a subject suffering from a cancer, wherein the cancer cells or proximate neovasculature express PSMA. Such cancers can include prostate, bladder, pancreas, lung, colon, kidney, melanomas and sarcomas. In a preferred embodiment the cancer cell is a prostate cancer cell. The cancer cells can be cells of a primary tumor or can be those of a metastatic tumor.

Another aspect of the invention provides an isolated antibody or an antigen-binding fragment thereof which specifically binds to an extracellular domain of PSMA wherein the antibody or the antigen-binding fragment thereof competitively inhibits the specific binding of a second antibody to its target epitope on PSMA, and wherein the second antibody is selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, 4.248.2, 4.360.3, 4.7.1, 4.4.1, 4.177.3, 4.16.1, 4.22.3, 4.28.3, 4.40.2, 4.48.3, 4.49.1, 4.209.3, 4.219.3, 4.288.1, 4.333.1, 4.54.1, 4.153.1, 4.232.3, 4.292.3, 4.304.1, 4.78.1, and 4.152.1.

Another aspect of the invention provides an isolated antibody or an antigen-binding fragment thereof that specifically binds to an epitope on PSMA defined by an antibody selected from the group consisting of PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, 4.248.2, 4.360.3, 4.7.1, 4.4.1, 4.177.3, 4.16.1, 4.22.3, 4.28.3, 4.40.2, 4.48.3, 4.49.1, 4.209.3, 4.219.3, 4.288.1, 4.333.1, 4.54.1, 4.153.1, 4.232.3, 4.292.3, 4.304.1, 4.78.1, and 4.152.1.

In particular embodiments, these antibodies are produced by hybridomas referred to herein as PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, Abgenix 4.248.2, Abgenix 4.360.3, Abgenix 4.7.1, Abgenix 4.4.1, Abgenix 4.177.3, Abgenix 4.16.1, Abgenix 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153.1, Abgenix 4.232.3, Abgenix 4.292.3, Abgenix 4.304.1, Abgenix 4.78.1, and Abgenix 4.152.1, respectively. These hybridomas were deposited with ATCC as an International Depository Authority and given the following Patent Deposit Designations (Table 1):

TABLE 1

| | | Patent | |
|---|---|---|---|
| | Hybridoma/ | Deposit | Date of |
| Antibody | Plasmid | Designation | Deposit |
| PSMA 3.7 | PSMA 3.7 | PTA-3257 | Apr. 5, 2001 |
| PSMA 3.9 | PSMA 3.9 | PTA-3258 | Apr. 5, 2001 |
| PSMA 3.11 | PSMA 3.11 | PTA-3269 | Apr. 10, 2001 |
| PSMA 5.4 | PSMA 5.4 | PTA-3268 | Apr. 10, 2001 |
| PSMA 7.1 | PSMA 7.1 | PTA-3292 | Apr. 18, 2001 |
| PSMA 7.3 | PSMA 7.3 | PTA-3293 | Apr. 18, 2001 |
| PSMA 10.3 | PSMA 10.3 | PTA-3347 | May 1, 2001 |
| | PSMA 10.3 | PTA-4413 | May 29, 2002 |
| | HC in | | |
| | pcDNA | | |
| | (SEQ ID NO: 7) | | |
| | PSMA 10.3 | PTA-4414 | May 29, 2002 |
| | Kappa in | | |
| | pcDNA | | |
| | (SEQ ID NO: 13) | | |
| PSMA 1.8.3 | PSMA 1.8.3 | PTA-3906 | Dec. 5, 2001 |
| PSMA A3.1.3 | PSMA A3.1.3 | PTA-3904 | Dec. 5, 2001 |
| PSMA A3.3.1 | PSMA A3.3.1 | PTA-3905 | Dec. 5, 2001 |
| Abgenix 4.248.2 | Abgenix 4.248.2 | PTA-4427 | Jun. 4, 2002 |
| Abgenix 4.360.3 | Abgenix 4.360.3 | PTA-4428 | Jun. 4, 2002 |
| Abgenix 4.7.1 | Abgenix 4.7.1 | PTA-4429 | Jun. 4, 2002 |
| Abgenix 4.4.1 | Abgenix 4.4.1 | PTA-4556 | Jul. 18, 2002 |
| Abgenix 4.177.3 | Abgenix 4.177.3 | PTA-4557 | Jul. 18, 2002 |
| Abgenix 4.16.1 | Abgenix 4.16.1 | PTA-4357 | May 16, 2002 |
| Abgenix 4.22.3 | Abgenix 4.22.3 | PTA-4358 | May 16, 2002 |
| Abgenix 4.28.3 | Abgenix 4.28.3 | PTA-4359 | May 16, 2002 |
| Abgenix 4.40.2 | Abgenix 4.40.2 | PTA-4360 | May 16, 2002 |
| Abgenix 4.48.3 | Abgenix 4.48.3 | PTA-4361 | May 16, 2002 |
| Abgenix 4.49.1 | Abgenix 4.49.1 | PTA-4362 | May 16, 2002 |
| Abgenix 4.209.3 | Abgenix 4.209.3 | PTA-4365 | May 16, 2002 |
| Abgenix 4.219.3 | Abgenix 4.219.3 | PTA-4366 | May 16, 2002 |
| Abgenix 4.288.1 | Abgenix 4.288.1 | PTA-4367 | May 16, 2002 |
| Abgenix 4.333.1 | Abgenix 4.333.1 | PTA-4368 | May 16, 2002 |
| Abgenix 4.54.1 | Abgenix 4.54.1 | PTA-4363 | May 16, 2002 |
| Abgenix 4.153.1 | Abgenix 4.153.1 | PTA-4388 | May 23, 2002 |
| Abgenix 4.232.3 | Abgenix 4.232.3 | PTA-4389 | May 23, 2002 |
| Abgenix 4.292.3 | Abgenix 4.292.3 | PTA-4390 | May 23, 2002 |
| Abgenix 4.304.1 | Abgenix 4.304.1 | PTA-4391 | May 23, 2002 |
| AB-PG1-XG1-006 | AB-PG1-XG1-006 | PTA-4403 | May 29, 2002 |
| | Heavy Chain | | |
| | (SEQ ID NO: 2) | | |
| | AB-PG1-XG1-006 | PTA-4404 | |
| | Light Chain | | |
| | (SEQ ID NO: 8) | | |
| AB-PG1-XG1-026 | AB-PG1-XG1-026 | PTA-4405 | May 29, 2002 |
| | Heavy Chain | | |
| | (SEQ ID NO: 3) | | |
| | AB-PG1-XG1-026 | PTA-4406 | |
| | Light Chain | | |
| | (SEQ ID NO: 9) | | |
| AB-PG1-XG1-051 | AB-PG1-XG1-051 | PTA-4407 | May 29, 2002 |
| | Heavy Chain | | |
| | (SEQ ID NO: 4) | | |
| | AB-PG1-XG1-051 | PTA-4408 | |
| | Light Chain | | |
| | (SEQ ID NO: 10) | | |
| AB-PG1-XG1-069 | AB-PG1-XG1-069 | PTA-4409 | May 29, 2002 |
| | Heavy Chain | | |
| | (SEQ ID NO: 5) | | |
| | AB-PG1-XG1-069 | PTA-4410 | |
| | Light Chain | | |
| | (SEQ ID NO: 11) | | |
| AB-PG1-XG1-077 | AB-PG1-XG1-077 | PTA-4411 | May 29, 2002 |
| | Heavy Chain | | |
| | (SEQ ID NO: 6) | | |
| | AB-PG1-XG1-077 | PTA-4412 | |
| | Light Chain | | |
| | (SEQ ID NO: 12) | | |

In another aspect of the invention, antibodies having particular sequences are provided. Specifically, the antibodies are selected from the group consisting of antibodies comprising: a heavy chain encoded by a nucleic acid molecule comprising the heavy chain coding region or regions of a nucleotide sequence selected from the group consisting of nucleotide sequences set forth as SEQ ID NOs: 2-7, and a light chain encoded by a nucleic acid molecule comprising the light chain coding region or regions of a nucleotide sequence selected from the group consisting of nucleotide sequences set forth as SEQ ID NOs: 8-13. Also provided are antigen-binding fragments of the foregoing antibodies.

The plasmids encoding the heavy and light chains of antibodies PSMA 10.3, AB-PG1-XG1-006, AB-PG1-XG1-026, AB-PG1-XG1-051, AB-PG1-XG1-069, AB-PG1-XG1-077 were also deposited with ATCC and are shown in Table 1 above. As used herein, the names of the deposited hybridomas or plasmids may be used interchangeably with the names of the antibodies. It would be clear to one of skill in the art when the name is intended to refer to the antibody or when it refers to the plasmids or hybridomas that encode or produce the antibodies, respectively. Additionally, the antibody names may be an abbreviated form of the name shown in Table 1. For instance antibody AB-PG1-XG1-006 may be referred to as AB-PG1-XG1-006, PG1-XG1-006, XG1-006, 006, etc. In another example, the antibody name PSMA 4.232.3 may be referred to as PSMA 4.232.1, 4.232.3, 4.232.1, 4.232, etc. It is intended that all of the variations in the name of the antibody refer to the same antibody and not a different one.

Antibodies are also provided that are encoded by particular sets of heavy and light chain sequences. In one embodiment an antibody (AB-PG1-XG1-006) encoded by a nucleic acid molecule which comprises the coding region or regions of the nucleic acid sequences set forth as :SEQ ID NOs: 2 and 8 is provided. In another embodiment the antibody (AB-PG1-XG1-026) is encoded by the nucleic acid molecules comprising the coding region or regions of nucleotide sequences set forth as: SEQ ID NOs: 3 and 9. In still another embodiment the antibody (AB-PG1-XG1-051) is encoded by the nucleic acid molecules comprising the coding region or regions of nucleotide sequences set forth as: SEQ ID NOs: 4 and 10. In yet another embodiment the antibody (AB-PG1-XG1-069) is encoded by the nucleic acid molecules comprising the coding region or regions of nucleotide sequences set forth as: SEQ ID NOs: 5 and 11. In another embodiment the antibody (AB-PG1-XG1-077) is encoded by the nucleic acid molecules comprising the coding region or regions of nucleotide sequences set forth as: SEQ ID NOs: 6 and 12. In yet another embodiment the antibody (PSMA 10.3) is encoded by the nucleic acid molecules comprising the coding region or regions of nucleotide sequences set forth as: SEQ ID NOs: 7 and 13.

In particularly preferred embodiments, the antibodies include a heavy chain variable region encoded by a nucleic acid molecule comprising the coding regions or regions of a nucleotide sequence selected from the group consisting of nucleotide sequences set forth as: SEQ ID NOs: 14, 18, 22, 26 and 30, and a light chain variable region encoded by a nucleic 10 acid molecule comprising the coding region or region of a nucleotide sequence selected from the group consisting of nucleotide sequences set forth as: SEQ ID NOs: 16, 20, 24, 28 and 32. As used herein, a “coding region” refers to a region of a nucleotide sequence that encodes a polypeptide sequence; the coding region can include a region coding for a portion of a protein that is later cleaved off, such as a signal peptide.

Those of skill in the art will appreciate that the invention includes nucleic acids and polypeptides that include nucleotide and amino acid sequences presented herein. In some instances, the nucleotide and amino acid sequences may include sequences that encode or that are signal peptides. The invention embraces each of these sequences with, or without, the portion of the sequence that encodes or is a signal peptide.

Antibodies also are provided that include particular sets of heavy and light chain variable sequences. In one embodiment an antibody (AB-PG1-XG1-006) includes an immunoglobulin variable sequence encoded by nucleic acid molecules which included the coding region or regions of the nucleic acid sequences set forth as :SEQ ID NOs: 14 and 16 is provided. Likewise the antibody may include an immunoglobulin variable sequence which comprises the amino acid sequences set forth as SEQ ID NOs: 15 and 17. In another embodiment the antibody (AB-PG1-XG1-026) includes an immunoglobulin variable sequence encoded by nucleic acid molecules comprising the coding region or regions of nucleotide sequences set forth as: SEQ ID NOs: 18 and 20 or includes an immunoglobulin variable sequence which comprises the amino acid sequences set forth as SEQ ID NOs: 19 and 21. In still another embodiment the antibody (AB-PG1-XG1-051) includes an immunoglobulin variable sequence encoded by the nucleic acid molecules comprising the coding region or regions of nucleotide sequences set forth as: SEQ ID NOs: 22 and 24 or includes an immunoglobulin variable sequence which comprises the amino acid sequences set forth as SEQ ID NOs: 23 and 25. In yet another embodiment the antibody (AB-PG1-XG1-069) includes an immunoglobulin variable sequence encoded by the nucleic acid molecules comprising the coding region or regions of nucleotide sequences set forth as: SEQ ID NOs: 26 and 28 or includes an immunoglobulin variable sequence which comprises the amino acid sequences set forth as SEQ ID NOs: 27 and 29. In another embodiment the antibody (AB-PG1-XG1-077) includes an immunoglobulin variable sequence encoded by the nucleic acid molecules comprising the coding region or regions of nucleotide sequences set forth as: SEQ ID NOs: 30 and 32 or includes an immunoglobulin variable sequence which comprises the amino acid sequences set forth as SEQ ID NOs: 31 and 33.

In certain embodiments, the antibody is encoded by a nucleic acid molecule that is highly homologous to the foregoing nucleic acid molecules. Preferably the homologous nucleic acid molecule comprises a nucleotide sequence that is at least about 90% identical to the nucleotide sequence provided herein. More preferably, the nucleotide sequence is at least about 95% identical, at least about 97% identical, at least about 98% identical, or at least about 99% identical to the nucleotide sequence provided herein. The homology can be calculated using various, publicly available software tools well known to one of ordinary skill in the art. Exemplary tools include the BLAST system available from the website of the National Center for Biotechnology Information (NCBI) at the National Institutes of Health.

One method of identifying highly homologous nucleotide sequences is via nucleic acid hybridization. Thus the invention also includes antibodies having the PSMA-binding properties and other functional properties described herein, which are encoded by nucleic acid molecules that hybridize under high stringency conditions to the foregoing nucleic acid molecules. Identification of related sequences can also be achieved using polymerase chain reaction (PCR) and other amplification techniques suitable for cloning related nucleic acid sequences. Preferably, PCR primers are selected to amplify portions of a nucleic acid sequence of interest, such as a CDR.

The term “high stringency conditions” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. One example of high-stringency conditions is hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68° C.

In other preferred embodiments, the antibodies include a heavy chain variable region comprising an amino acid sequence selected from the group consisting of amino acid sequences set forth as: SEQ ID NOs: 15, 19, 23, 27 and 31, and a light chain variable region comprising an amino acid sequence selected from the group consisting of nucleotide sequences set forth as: SEQ ID NOs: 17, 21, 25, 29 and 33. Antigen-binding fragments of the foregoing also are provided, as described elsewhere herein.

As used herein, the term “antibody” refers to a glycoprotein comprising at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody as used herein, refers to one or more portions of an antibody that retain the ability to specifically bind to an antigen (e.g., PSMA). It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546) which consists of a V_{H} domain; and (vi) an isolated complementarity determining region (CDR). Furthermore, although the two domains of the Fv fragment, V and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

An “isolated antibody”, as used herein, is intended to refer to an antibody which is substantially free of other antibodies having different antigenic specificities (e.g., an isolated antibody that specifically binds to PSMA is substantially free of antibodies that specifically bind antigens other than PSMA). An isolated antibody that specifically binds to an epitope, isoform or variant of PSMA may, however, have cross-reactivity to other related antigens, e.g., from other species (e.g., PSMA species homologs). Moreover, an isolated antibody may be substantially free of other cellular material and/or chemicals. As used herein, “specific binding” refers to antibody binding to a predetermined antigen. Typically, the antibody binds with an affinity that is at least two-fold greater than its affinity for binding to a non-specific antigen (e.g., BSA, casein) other than the predetermined antigen or a closely-related antigen.

The isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g. IgM or IgG1) that is encoded by heavy chain constant region genes. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment, and a Fv fragment.

The antibodies of the present invention can be polyclonal, monoclonal, or a mixture of polyclonal and monoclonal antibodies. The antibodies can be produced by a variety of techniques well known in the art. Procedures for raising polyclonal antibodies are well known. For example anti-PSMA polyclonal antibodies are raised by administering PSMA protein subcutaneously to New Zealand white rabbits which have first been bled to obtain pre-immune serum. The PSMA can be injected at a total volume of 100 μl per site at six different sites, typically with one or more adjustments. The rabbits are then bled two weeks after the first injection and periodically boosted with the same antigen three times every six weeks. A sample of serum is collected 10 days after each boost. Polyclonal antibodies are recovered from the serum, preferably by affinity chromatography using PSMA to capture the antibody. This and other procedures for raising polyclonal antibodies are disclosed in E. Harlow, et. al., editors, Antibodies: A Laboratory Manual (1988), which is hereby incorporated by reference.

Monoclonal antibody production may be effected by techniques which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

Mammalian lymphocytes typically are immunized by in vivo immunization of the animal (e.g., a mouse) with the desired protein or polypeptide, e.g., with PSMA in the present invention. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. Mouse lymphocytes give a higher percentage of stable fusions with the mouse myeloma lines described herein. Of these, the BALB/c mouse is preferred. However, other mouse strains, rabbit, hamster, sheep and frog may also be used as hosts for preparing antibody-producing cells. See; Goding (in Monoclonal Antibodies: Principles and Practice, 2d ed., pp. 60-61, Orlando, Fla., Academic Press, 1986). In particular, mouse strains that have human immunoglobulin genes inserted in the genome (and which cannot produce mouse immunoglobulins) are preferred. Examples include the HuMAb mouse strains produced by Medarex/GenPharm International, and the XenoMouse strains produced by Abgenix. Such mice produce fully human immunoglobulin molecules in response to immunization.

Those antibody-producing cells that are in the dividing plasmablast stage fuse preferentially. Somatic cells may be obtained from the lymph nodes, spleens and peripheral blood of antigen-primed animals, and the lymphatic cells of choice depend to a large extent on their empirical usefulness in the particular fusion system. The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Alternatively, human somatic cells capable of producing antibody, specifically B lymphocytes, are suitable for fusion with myeloma cell lines. While B lymphocytes from biopsied spleens, tonsils or lymph nodes of an individual may be used, the more easily accessible peripheral blood B lymphocytes are preferred. The lymphocytes may be derived from patients with diagnosed prostate carcinomas or another PSMA-expressing cancer. In addition, human B cells may be directly immortalized by the Epstein-Barr virus (Cole et al., 1995, Monoclonal Antibodies and Cancer Therapy, Alan R. Liss, Inc., pp. 77-96). Although somatic cell hybridization procedures are preferred, in principle, other techniques for producing monoclonal antibodies can be employed such as viral or oncogenic transformation of B lymphocytes.

Myeloma cell lines suited for use in hybridoma-producing fusion procedures preferably are non-antibody-producing, have high fusion efficiency, and enzyme deficiencies that render them incapable of growing in certain selective media which support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elseview, 1984).

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference).

In other embodiments, the antibodies can be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the invention.

According to an alternative embodiment, the monoclonal antibodies of the present invention can be modified to be in the form of a bispecific antibody, or a multispecific antibody. The term “bispecific antibody” is intended to include any agent, e.g., a protein, peptide, or protein or peptide complex, which has two different binding specificities which bind to, or interact with (a) a cell surface antigen and (b) an Fc receptor on the surface of an effector cell. The term “multispecific antibody” is intended to include any agent, e.g., a protein, peptide, or protein or peptide complex, which has more than two different binding specificities which bind to, or interact with (a) a cell surface antigen, (b) an Fc receptor on the surface of an effector cell, and (c) at least one other component. Accordingly, the invention includes, but is not limited to, bispecific, trispecific, tetraspecific, and other multispecific antibodies which are directed to cell surface antigens, such as PSMA, and to Fc receptors on effector cells. The term “bispecific antibodies” further includes diabodies. Diabodies are bivalent, bispecific antibodies in which the V_{H} and V_{L} domains are expressed on a single polypeptide chain, but using a linker that is too short to allow for pairing between the two domains on the same chain, thereby forcing the domains to pair with complementary domains of another chain and creating two antigen-binding sites (see e.g., Holliger, P., et al. (1993) Proc. Natl. Acad. Sci. USA 90:6444-6448; Poijak, R. J., et al. (1994) Structure 2:1121-1123).

A bispecific antibody can be formed of an antigen-binding region specific for the extracellular domain of PSMA and an antigen-binding region specific for an effector cell which has tumoricidal or tumor inhibitory activity. The two antigen-binding regions of the bispecific antibody are either chemically linked or can be expressed by a cell genetically engineered to produce the bispecific antibody. (See generally, Fanger et al., 1995 Drug News & Perspec. 8(3):133-137). Suitable effector cells having tumoricidal activity include but are not limited to cytotoxic T-cells (primarily CD8⁺ cells), natural killer cells, etc. An effective amount of a bispecific antibody according to the invention is administered to a prostrate cancer patient and the bispecific antibody kills and/or inhibits proliferation of the malignant cells after localization at sites of primary or metastatic tumors bearing PSMA.

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Human antibodies directed against PSMA are generated using transgenic mice carrying parts of the human immune system rather than the mouse system.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans.

Preferably, the mice are 6-16 weeks of age upon the first immunization. For example, a purified or enriched preparation of PSMA antigen (e.g., recombinant PSMA, PSMA-expressing cells, dimeric PSMA) is used to immunize the mice intraperitoneally (IP), although other routes of immunization known to one of ordinary skill in the art are also possible. PSMA antigen is injected in combination with an adjuvant, such as complete Freund's adjuvant, and preferably the initial injection is followed by booster immunizations with antigen in an adjuvant, such as incomplete Freund's adjuvant. The immune response is monitored over the course of the immunization protocol with plasma samples obtained by, for example, retroorbital bleeds. The plasma is screened by ELISA (as described below), and mice with sufficient titers of anti-PSMA human immunoglobulin are used for fusions. Mice are boosted intravenously with antigen 3 days before sacrifice and removal of the spleen.

In particular embodiments, the antibodies are produced by hybridomas referred to herein as PSMA 3.7 (PTA-3257), PSMA 3.8, PSMA 3.9 (PTA-3258), PSMA 3.11 (PTA-3269), PSMA 5.4 (PTA-3268), PSMA 7.1 (PTA-3292), PSMA 7.3 (PTA-3293), PSMA 10.3 (PTA-3247), PSMA 1.8.3 (PTA-3906), PSMA A3.1.3 (PTA-3904), PSMA A3.3.1 (PTA-3905), Abgenix 4.248.2 (PTA-4427), Abgenix 4.360.3 (PTA-4428), Abgenix 4.7.1 (PTA-4429), Abgenix 4.4.1 (PTA-4556), Abgenix 4.177.3 (PTA-4557), Abgenix 4.16.1 (PTA-4357), Abgenix 4.22.3 (PTA-4358), Abgenix 4.28.3 (PTA-4359), Abgenix 4.40.2 (PTA-4360), Abgenix 4.48.3 (PTA-4361), Abgenix 4.49.1 (PTA-4362), Abgenix 4.209.3 (PTA-4365), Abgenix 4.219.3 (PTA-4366), Abgenix 4.288.1 (PTA-4367), Abgenix 4.333.1 (PTA-4368), Abgenix 4.54.1 (PTA-4363), Abgenix 4.153.1 (PTA-4388), Abgenix 4.232.3 (PTA-4389), Abgenix 4.292.3 (PTA-4390), Abgenix 4.304.1 (PTA-4391), Abgenix 4.78.1 (PTA-4652), and Abgenix 4.152.1 (PTA-4653). These hybridomas were deposited pursuant to, and in satisfaction of, the requirements of the Budapest Treaty on the International Recognition of the Deposit of Microorganisms for the Purposes of Patent Procedure with the American Type Culture Collection (“ATCC”) as an International Depository Authority and given the Patent Deposit Designations shown above and in Table 1.

The present invention further provides nucleic acid molecules encoding anti-PSMA antibodies and vectors comprising the nucleic acid molecules as described herein. The vectors provided can be used to transform or transfect host cells for producing anti-PSMA antibodies with the specificity of antibodies described herein. In a preferred embodiment the antibodies produced will have the specificity of the antibodies AB-PG1-XG1-006, AB-PG1-XG1-026, AB-PG1-XG1-051, AB-PG1, XG1-069, AB-PG1-XG1-077 and PSMA 10.3. In one embodiment the vectors can comprise an isolated nucleic acid molecule encoding the heavy chain of the antibodies listed above encoded by a nucleic acid molecules comprising the coding region or regions of the nucleic acid sequences set forth as SEQ ID NO: 2-7. In another embodiment, the vectors can comprise the nucleic acid sequences encoding the light chain of the antibodies set forth as SEQ ID NOs: 8-13. In a further embodiment the vectors of the invention may comprise a heavy chain and a light chain sequence. In a further embodiment, plasmids are given which produce the antibodies or antigen binding fragments described herein. Plasmids of the invention include plasmids selected from the group consisting of: AB-PG1-XG1-006 Heavy Chain (SEQ ID NO: 2), AB-PG1-XG1-006 Light Chain (SEQ ID NO: 8), AB-PG1-XG1-026 Heavy Chain (SEQ ID NO: 3), AB-PG1-XG1-026 Light Chain (SEQ ID NO: 9), AB-PG1-XG1-051 Heavy Chain (SEQ ID NO: 4), AB-PG1-XG1-051 Light Chain (SEQ ID NO: 10), AB-PG1-XG1-069 Heavy Chain (SEQ ID NO: 5), AB-PG1-XG1-069 Light Chain (SEQ ID NO: 11), AB-PG1-XG1-077 Heavy Chain (SEQ ID NO: 6), AB-PG1-XG1-077 Light Chain (SEQ ID NO: 12), PSMA 10.3 Heavy Chain (SEQ ID NO: 7), and PSMA 10.3 Kappa (SEQ ID NO: 13).

The isolated antibody or antigen-binding fragment thereof preferably is selected for its ability to bind live cells expressing PSMA. In order to demonstrate binding of monoclonal antibodies to live cells expressing the PSMA, flow cytometry can be used. For example, cell lines expressing PSMA (grown under standard growth conditions) or prostate cancer cells that express PSMA are mixed with various concentrations of monoclonal antibodies in PBS containing 0.1% Tween 80 and 20% mouse serum, and incubated at 37° C. for 1 hour. After washing, the cells are reacted with fluorescein-labeled anti-human IgG secondary antibody (if human anti-PSMA antibodies were used) under the same conditions as the primary antibody staining. The samples can be analyzed by a fluorescence activated cell sorter (FACS) instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

Binding of the antibody or antigen-binding fragment thereof to live cells expressing PSMA can inhibit the growth of the cells or mediate cytolysis of the cells. Cytolysis can be complement mediated or can be mediated by effector cells. In a preferred embodiment, the cytolysis is carried out in a living organism, preferably a mammal, and the live cell is a tumor cell. Examples of tumors which can be targeted by the antibodies of the invention include, any tumor that expresses PSMA, such as, prostate, bladder, pancreas, lung, colon, kidney, melanomas and sarcomas. In a preferred embodiment the tumor cell is a prostate cancer cell.

The testing of antibody cytolytic activity in vitro by chromium release assay can provide an initial screening prior to testing in vivo models. This testing can be carried out using standard chromium release assays. Briefly, polymorphonuclear cells (PMN), or other effector cells, from healthy donors can be purified by Ficoll Hypaque density centrifugation, followed by lysis of contaminating erythrocytes. Washed PMNs can be suspended in RPMI supplemented with 10% heat-inactivated fetal calf serum and mixed with ⁵¹Cr labeled cells expressing PSMA, at various ratios of effector cells to tumor cells (effector cells:tumor cells). Purified anti-PSMA IgGs can then be added at various concentrations. Irrelevant IgG can be used as negative control. Assays can be carried out for 0-120 minutes at 37° C. Samples can be assayed for cytolysis by measuring ⁵¹Cr release into the culture supernatant. Anti-PSMA monoclonal antibodies can also be tested in combinations with each other to determine whether cytolysis is enhanced with multiple monoclonal antibodies.

Antibodies which bind to PSMA also can be tested in an in vivo model (e.g., in mice) to determine their efficacy in mediating cytolysis and killing of cells expressing PSMA, e.g., tumor cells. These antibodies can be selected, for example, based on the following criteria, which are not intended to be exclusive:

1) binding to live cells expressing PSMA;

2) high affinity of binding to PSMA;

3) binding to a unique epitope on PSMA (to eliminate the possibility that antibodies with complimentary activities when used in combination would compete for binding to the same epitope);

4) opsonization of cells expressing PSMA;

5) mediation of growth inhibition, phagocytosis and/or killing of cells expressing PSMA in the presence of effector cells;

6) modulation (inhibition or enhancement) of NAALADase, folate hydrolase, dipeptidyl peptidase IV and/or γ-glutamyl hydrolase activities;

7) growth inhibition, cell cycle arrest and/or cytotoxicity in the absence of effector cells;

8) internalization of PSMA;

9) binding to a conformational epitope on PSMA;

10) minimal cross-reactivity with cells or tissues that do not express PSMA; and

11) preferential binding to dimeric forms of PSMA rather than monomeric forms of PSMA.

Preferred antibodies of the invention meet one or more, and preferably all, of these criteria. In a particular embodiment, the antibodies are used in combination, e.g., as a pharmaceutical composition comprising two or more different anti-PSMA antibodies or binding fragments thereof. For example, anti-PSMA antibodies having different, but complementary activities can be combined in a single therapy to achieve a desired therapeutic or diagnostic effect. An illustration of this would be a composition containing an anti-PSMA antibody that mediates highly effective killing of target cells in the presence of effector cells, combined with another anti-PSMA antibody that inhibits the growth of cells expressing PSMA.

In a preferred aspect of the invention, the antibody or antigen-binding fragment thereof binds to a conformational epitope within the extracellular domain of the PSMA molecule. To determine if the selected human anti-PSMA antibodies bind to conformational epitopes, each antibody can be tested in assays using native protein (e.g., non-denaturing immunoprecipitation, flow cytometric analysis of cell surface binding) and denatured protein (e.g., Western blot, immunoprecipitation of denatured proteins). A comparison of the results will indicate whether the antibodies bind conformational epitopes. Antibodies that bind to native protein but not denatured protein are those antibodies that bind conformational epitopes, and are preferred antibodies.

In another preferred aspect of the invention, the antibody or antigen-binding fragment thereof binds to a dimer-specific epitope on PSMA. Generally, antibodies or antigen-binding fragments thereof which bind to a dimer-specific epitope preferentially bind the PSMA dimer rather than the PSMA monomer. To determine if the selected human anti-PSMA antibodies bind preferentially (i.e., selectively and/or specifically) to a PSMA dimer, each antibody can be tested in assays (e.g., immunoprecipitation followed by Western blotting) using native dimeric PSMA protein and dissociated monomeric PSMA protein. A comparison of the results will indicate whether the antibodies bind preferentially to the dimer or to the monomer. Antibodies that bind to the PSMA dimer but not to the monomeric PSMA protein are preferred antibodies.

Preferred antibodies include antibodies that competitively inhibit the specific binding of a second antibody to its target epitope on PSMA. To determine competitive inhibition, a variety of assays known to one of ordinary skill in the art can be employed. For example, the cross-competition assays set forth in Examples 4 and 21 can be used to determine if an antibody competitively inhibits binding to PSMA by another antibody. These examples provide cell-based methods employing flow cytometry or solid phase binding analysis. Other assays that evaluate the ability of antibodies to cross-compete for PSMA molecules that are not expressed on the surface of cells, in solid phase or in solution phase, also can be used. These assays preferably use the PSMA multimers described herein.

Certain preferred antibodies competitively inhibit the specific binding of a second antibody to its target epitope on PSMA by at least about 10%, 20%, 30%, 40%, 50%, 60%, 70%, 80%, 90%, 95%, or 99%. Inhibition can be assessed at various molar ratios or mass ratios; for example competitive binding experiments can be conducted with a 2-fold, 3-fold, 4-fold, 5-fold, 7-fold, 10-fold or more molar excess of the first antibody over the second antibody.

Other preferred antibodies include antibodies that specifically (i.e., selectively) bind to an epitope on PSMA defined by a second antibody. To determine the epitope, one can use standard epitope mapping methods known in the art. For example, fragments (peptides) of PSMA antigen (preferably synthetic peptides) that bind the second antibody can be used to determine whether a candidate antibody binds the same epitope. For linear epitopes, overlapping peptides of a defined length (e.g., 8 or more amino acids) are synthesized. The peptides preferably are offset by 1 amino acid, such that a series of peptides covering every 8 amino acid fragment of the PSMA protein sequence are prepared. Fewer peptides can be prepared by using larger offsets, e.g., 2 or 3 amino acids. In addition, longer peptides (e.g., 9-, 10- or 11-mers) can be synthesized. Binding of peptides to antibodies can be determined using standard methodologies including surface plasmon resonance (BIACORE; see Example 22) and ELISA assays. For examination of conformational epitopes, larger PSMA fragments can be used. Other methods that use mass spectrometry to define conformational epitopes have been described and can be used (see, e.g., Baerga-Ortiz et al., Protein Science 11: 1300-1308, 2002 and references cited therein). Still other methods for epitope determination are provided in standard laboratory reference works, such as Unit 6.8 (“Phage Display Selection and Analysis of B-cell Epitopes”) and Unit 9.8 (“Identification of Antigenic Determinants Using Synthetic Peptide Combinatorial Libraries”) of Current Protocols in Immunology, Coligan et al., eds., John Wiley & Sons. Epitopes can be confirmed by introducing point mutations or deletions into a known epitope, and then testing binding with one or more antibodies to determine which mutations reduce binding of the antibodies.

In one embodiment of the invention the antibody or antigen-binding fragment thereof binds to and is internalized with PSMA expressed on cells. The mechanism by which the antibody or antigen-binding fragment thereof is internalized with the prostate specific membrane antigen is not critical to the practice of the present invention. For example, the antibody or antigen-binding fragment thereof can induce internalization of PSMA. Alternatively, internalization of the antibody or antigen-binding fragment thereof can be the result of routine internalization of PSMA. The antibody or antigen-binding fragment thereof can be used in an unmodified form, alone or in combination with other compositions. Alternatively, the antibody or antigen-binding fragment thereof can be bound to a substance effective to kill the cells upon binding of the antibody or antigen-binding fragment thereof to prostate specific membrane antigen and upon internalization of the biological agent with the prostate specific membrane antigen.

The human PSMA antibodies of the present invention specifically bind cell-surface PSMA and/or rsPSMA with sub-nanomolar affinity. The human PSMA antibodies of the present invention have binding affinities of about 1×10⁻⁹M or less, preferably about 1×10⁻¹⁰M or less, more preferably 1×10⁻¹¹M or less. In a particular embodiment the binding affinity is less than about 5×10⁻¹⁰M.

An antibody can be linked to a detectable marker, an antitumor agent or an immunomodulator. Antitumor agents can include cytotoxic agents and agents that act on tumor neovasculature. Detectable markers include, for example, radioactive or fluorescent markers. Cytotoxic agents include cytotoxic radionuclides, chemical toxins and protein toxins.

The cytotoxic radionuclide or radiotherapeutic isotope preferably is an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide may a beta-emitting isotope such as ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents that may be conjugated to the anti-PSMA antibodies of the present invention include dolastatins (U.S. Pat. Nos. 6,034,065 and 6,239,104) and derivatives thereof. Of particular interest is dolastatin 10 (dolavaline-valine-dolaisoleuine-dolaproine-dolaphenine) and the derivatives auristatin PHE (dolavaline-valine-dolaisoleuine-dolaproine-phenylalanine-methyl ester) (Pettit, G. R. et al., Anticancer Drug Des. 13(4):243-277, 1998; Woyke, T. et al., Antimicrob. Agents Chemother. 45(12):3580-3584, 2001), and aurastatin E and the like. Toxins that are less preferred in the compositions and methods of the invention include poisonous lectins, plant toxins such as ricin, abrin, modeccin, botulina and diphtheria toxins. Of course, combinations of the various toxins could also be coupled to one antibody molecule thereby accommodating variable cytotoxicity. Other chemotherapeutic agents are known to those skilled in the art.

Toxin-conjugated forms of the PSMA antibodies of the present invention mediate specific cell killing of PSMA-expressing cells at picomolar concentrations. The toxin-conjugated PSMA antibodies of the present invention exhibit IC₅₀s at concentrations of less than about 1×10⁻¹⁰M, preferably less than about 1×10⁻¹¹M, more preferably less than about 1×10⁻¹²M. In a particular embodiment an IC₅₀ is achieved at a concetration of less than about 1.5×10⁻¹¹M.

Agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein) and interferon inducible protein 10 (U.S. Pat. No. 5,994,292). A number of antiangiogenic agents currently in clinical trials are also contemplated. Agents currently in clinical trials include: 2ME2, Angiostatin, Angiozyme, Anti-VEGF RhuMAb, Apra (CT-2584), Avicine, Benefin, BMS275291, Carboxyamidotriazole, CC4047, CC5013, CC7085, CDC801, CGP-41251 (PKC 412), CM11, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD1839), Marimastat, Metastat (Col-3), Neovastat, Octreotide, Paclitaxel, Penicillamine, Photofrin, Photopoint, PI-88, Prinomastat (AG-3340), PTK787 (ZK22584), RO317453, Solimastat, Squalamine, SU 101, SU 5416, SU-6668, Suradista (FCE 26644), Suramin (Metaret), Tetrathiomolybdate, Thalidomide, TNP-470 and Vitaxin. additional antiangiogenic agents are described by Kerbel, J. Clin. Oncol. 19(18s):45s-51s, 2001, which is incorporated by reference herein. Immunomodulators suitable for conjugation to anti-PSMA antibodies include α-interferon, γ-interferon, and tumor necrosis factor alpha (TNFα).

The coupling of one or more toxin molecules to the anti-PSMA antibody is envisioned to include many chemical mechanisms, for instance covalent binding, affinity binding, intercalation, coordinate binding, and complexation. The toxic compounds used to prepare the anti-PSMA immunotoxins are attached to the antibodies or PSMA-binding fragments thereof by standard protocols known in the art.

The covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In preferred embodiments, it is contemplated that one may wish to first derivatize the antibody, and then attach the toxin component to the derivatized product. Suitable cross-linking agents for use in this manner include, for example, SPDP (N-succinimidyl-3-(2-pyridyldithio)propionate), and SMPT, 4-succinimidyl-oxycarbonyl-methyl-(2-pyridyldithio)toluene.

In addition, protein toxins can be fused to the anti-PSMA antibody or PSMA binding fragment by genetic methods to form a hybrid immunotoxin fusion protein. To make a fusion immunotoxin protein in accordance with the invention, a nucleic acid molecule is generated that encodes an anti-PSMA antibody, a fragment of an anti-PSMA antibody, a single chain anti-PSMA antibody, or a subunit of an anti-PSMA antibody linked to a protein toxin. Such fusion proteins contain at least a targeting agent (e.g., anti-PSMA antibody subunit) and a toxin of the invention, operatively attached. The fusion proteins may also include additional peptide sequences, such as peptide spacers which operatively attach the targeting agent and toxin compound, as long as such additional sequences do not appreciably affect the targeting or toxin activities of the fusion protein. The two proteins can be attached by a peptide linker or spacer, such as a glycine-serine spacer peptide, or a peptide hinge, as is well known in the art. Thus, for example, the C-terminus of an anti-PSMA antibody or fragment thereof can be fused to the N-terminus of the protein toxin molecule to form an immunotoxin that retains the binding properties of the anti-PSMA antibody. Other fusion arrangements will be known to one of ordinary skill in the art.

To express the fusion immunotoxin, the nucleic acid encoding the fusion protein is inserted into an expression vector in accordance with standard methods, for stable expression of the fusion protein, preferably in mammalian cells, such as CHO cells. The fusion protein can be isolated and purified from the cells or culture supernatant using standard methodology, such as a PSMA affinity column.

Radionuclides typically are coupled to an antibody by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the diethylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed p-SCN-Bz-HEHA (1,4,7,10,13,16-hexaazacyclo-octadecane-N,N′,N″,N′″,N″″,N″″-hexaacetic acid) (Deal et al., J. Med. Chem. 42:2988, 1999), which is an effective chelator of radiometals such as ²²⁵Ac. Yet another example is DOTA (1,4,7,10-tetraazacyclododecane N,N′,N″,N′″-tetraacetic acid), which is a bifunctional chelating agent (see McDevitt et al., Science 294:1537-1540, 2001) that can be used in a two-step method for labeling followed by conjugation.

In another aspect, the invention provides compositions comprising a multimeric (e.g., dimeric) PSMA protein, an isolated antibody, an antibody derivatized or linked to other functional moieties, or an antigen-binding fragment thereof or a combination of one or more of the aforementioned multimeric PSMA proteins, antibodies or antigen-binding fragments thereof. The compositions include a physiologically or pharmaceutically acceptable carrier, excipient, or stabilizer mixed with the isolated multimeric PSMA protein, antibody or antigen-binding fragment thereof. In a preferred embodiment, the compositions include a combination of multiple (e.g., two or more) isolated multimeric PSMA proteins, antibodies or antigen-binding portions thereof of the invention. Preferably, each of the antibodies or antigen-binding portions thereof of the composition binds to a distinct conformational epitope of PSMA. In one embodiment, anti-PSMA antibodies having complementary activities are used in combination, e.g., as a pharmaceutical composition, comprising two or more anti-PSMA antibodies. For example, an antibody that mediates highly effective cytolysis of target cells in the presence of effector cells can be combined with another antibody that inhibits the growth of cells expressing PSMA. As used herein, “target cell” shall mean any undesirable cell in a subject (e.g., a human or animal) that can be targeted by a composition of the invention. In preferred embodiments, the target cell is a cell expressing or overexpressing PSMA. Cells expressing PSMA typically include tumor cells, such as prostate, bladder, pancreas, lung, kidney, colon tumor cells, melanomas, and sarcomas.

Pharmaceutical compositions of the invention also can be administered in combination therapy, i.e., combined with other agents. For example, the combination therapy can include a composition of the present invention with at least one anti-tumor agent, immunomodulator, immunostimulatory agent, or other conventional therapy. The agent may be bound or conjugated to or formed as a recombinant fusion molecule with the PSMA antibodies of the present invention for directed targeting of the agent to PSMA-expressing cells.

The PSMA antibodies of the present invention may be used as a targeting moiety for delivery of replication-selective virus to PSMA-expressing cells for tumor therapy. Replication-competent virus such as the p53 pathway targeting adenovirus mutant dll520, ONYX-015, kill tumor cells selectively (Biederer, C. et al., J. Mol. Med. 80(3):163-175, 2002).

The compositions of the present invention may include or be diluted into a pharmaceutically-acceptable carrier. As used herein, “pharmaceutically acceptable carrier” or “physiologically acceptable carrier” means one or more compatible solid or liquid fillers, diluents or encapsulating substances which are suitable for administration to a human or other mammal such as a primate, dog, cat, horse, cow, sheep, or goat. Such carriers include any and all salts, solvents, dispersion media, coatings, antibacterial and antifungal agents, isotonic and absorption delaying agents, and the like that are physiologically compatible. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Preferably, the carrier is suitable for oral, intranasal, intravenous, intramuscular, subcutaneous, parenteral, spinal, intradermal or epidermal administration (e.g., by injection or infusion). Suitable carriers can be found in Remington's Pharmaceutical Sciences, Mack Publishing Company, Easton, Pa. Depending on the route of administration, the active compound, i.e., antibody or PSMA multimer may be coated in a material to protect the compound from the action of acids and other natural conditions that may inactivate the compound.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. The components of the pharmaceutical compositions also are capable of being co-mingled with the molecules of the present invention, and with each other, in a manner such that there is no interaction which would substantially impair the desired pharmaceutical efficacy. Such preparations may routinely contain salts, buffering agents, preservatives, compatible carriers, and optionally other therapeutic agents, such as supplementary immune potentiating agents including adjuvants, chemokines and cytokines. When used in medicine, the salts should be pharmaceutically acceptable, but non-pharmaceutically acceptable salts may conveniently be used to prepare pharmaceutically-acceptable salts thereof and are not excluded from the scope of the invention.

A salt retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The pharmaceutical preparations of the invention also may include isotonicity agents. This term is used in the art interchangeably with iso-osmotic agent, and is known as a compound which is added to the pharmaceutical preparation to increase the osmotic pressure to that of 0.9% sodium chloride solution, which is iso-osmotic with human extracellular fluids, such as plasma. Preferred isotonicity agents are sodium chloride, mannitol, sorbitol, lactose, dextrose and glycerol.

Optionally, the pharmaceutical preparations of the invention may further comprise a preservative, such as benzalkonium chloride. Suitable preservatives also include but are not limited to: chlorobutanol (0.3-0.9% W/V), parabens (0.01-5.0%), thimerosal (0.004-0.2%), benzyl alcohol (0.5-5%), phenol (0.1-1.0%), and the like.

The formulations provided herein also include those that are sterile. Sterilization processes or techniques as used herein include aseptic techniques such as one or more filtration (0.45 or 0.22 micron filters) steps.

An anti-PSMA antibody composition may be combined, if desired, with a pharmaceutically-acceptable carrier.

The pharmaceutical compositions may contain suitable buffering agents, including: acetic acid in a salt; citric acid in a salt; boric acid in a salt; and phosphoric acid in a salt.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of PSMA multimers and/or anti-PSMA antibodies, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulations suitable for oral, subcutaneous, intravenous, intramuscular, etc. administration can be found in Remington 's Pharmaceutical Sciences, Mack Publishing Co., Easton, Pa.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The therapeutics of the invention can be administered by any conventional route, including injection or by gradual infusion over time. The administration may, for example, be oral, intravenous, intraperitoneal, intramuscular, intracavity, intratumor, or transdermal. When antibodies are used therapeutically, preferred routes of administration include intravenous and by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing antibody aerosols without resorting to undue experimentation.

The pharmaceutical preparations of the invention, when used in alone or in cocktails, are administered in therapeutically effective amounts. Effective amounts are well known to those of ordinary skill in the art and are described in the literature. A therapeutically effective amount will be determined by the parameters discussed below; but, in any event, is that amount which establishes a level of the drug(s) effective for treating a subject, such as a human subject, having one of the conditions described herein. An effective amount means that amount alone or with multiple doses, necessary to delay the onset of, inhibit completely or lessen the progression of or halt altogether the onset or progression of the condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

An “effective amount” is that amount of an anti-PSMA antibody or PSMA multimer that alone, or together with further doses, produces the desired response, e.g. treats a malignancy in a subject. This may involve only slowing the progression of the disease temporarily, although more preferably, it involves halting the progression of the disease permanently. This can be monitored by routine methods. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of anti-PSMA antibodies or PSMA multimers for producing the desired response in a unit of weight or volume suitable for administration to a patient. The response can, for example, be measured by determining the physiological effects of the anti-PSMA antibody or PSMA multimer, such as regression of a tumor or decrease of disease symptoms. Other assays will be known to one of ordinary skill in the art and can be employed for measuring the level of the response.

The doses of anti-PSMA antibodies or PSMA multimers administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, or infusion.

In general, doses can range from about 10 μg/kg to about 100,000 μg/kg. Based upon the composition, the dose can be delivered continuously, such as by continuous pump, or at periodic intervals. Desired time intervals of multiple doses of a particular composition can be determined without undue experimentation by one skilled in the art. Other protocols for the administration of anti-PSMA antibody or PSMA multimers will be known to one of ordinary skill in the art, in which the dose amount, schedule of administration, sites of administration, mode of administration and the like vary from the foregoing.

Dosage may be adjusted appropriately to achieve desired drug levels, locally or systemically. Generally, daily oral doses of active compounds will be from about 0.1 mg/kg per day to 30 mg/kg per day. It is expected that IV doses in the range of 0.01-1.00 mg/kg will be effective. In the event that the response in a subject is insufficient at such doses, even higher doses (or effective higher doses by a different, more localized delivery route) may be employed to the extent that patient tolerance permits. Continuous IV dosing over, for example, 24 hours or multiple doses per day also are contemplated to achieve appropriate systemic levels of compounds.

In general, doses of radionuclide delivered by the anti-PSMA antibodies of the invention can range from about 0.01 mCi/Kg to about 10 mCi/kg. Preferably the dose of radionuclide ranges from about 0.1 mCi/Kg to about 1.0 mCi/kg. The optimal dose of a given isotope can be determined empirically by simple routine titration experiments well known to one of ordinary skill in the art.

Administration of anti-PSMA antibody or PSMA multimer compositions to mammals other than humans, e.g. for testing purposes or veterinary therapeutic purposes, is carried out under substantially the same conditions as described above.

The compositions (antibodies to PSMA and derivatives/conjugates thereof and PSMA multimers) of the present invention have in vitro and in vivo diagnostic and therapeutic utilities. For example, these molecules can be administered to cells in culture, e.g. in vitro or ex vivo, or in a subject, e.g., in vivo, to treat, prevent or diagnose a variety of disorders. As used herein, the term “subject” is intended to include humans and non-human animals. Preferred subjects include a human patient having a disorder characterized by expression, typically aberrant expression (e.g., overexpression) of PSMA. Other preferred subjects include subjects that are treatable with the compositions of the invention. This includes those who have or are at risk of having a cancer or who would otherwise would benefit from an enhanced or elicited immune response to cells expressing PSMA. In preferred embodiments these cells express PSMA on their surface.

One aspect of the present invention relates to a method of detecting cancerous cells or portions thereof in a biological sample (e.g., histological or cytological specimens, biopsies and the like), and, in particular, to distinguish malignant tumors from normal tissues and non-malignant tumors. This method involves providing an antibody or an antigen-binding binding fragment thereof, probe, or ligand, which binds to an extracellular domain of PSMA of such cells, e.g., an anti-PSMA antibody. The anti-PSMA antibody is bound to a label that permits the detection of the cells or portions thereof (e.g., PSMA or fragments thereof liberated from such cancerous cells) upon binding of the anti-PSMA antibody to the cells or portions thereof. The biological sample is contacted with the labeled anti-PSMA antibody under conditions effective to permit binding of the anti-PSMA antibody to the extracellular domain of PSMA of any of the cells or portions thereof in the biological sample. The presence of any cells or portions thereof in the biological sample is detected by detection of the label. In one preferred form, the contact between the anti-PSMA antibody and the biological sample is carried out in a living mammal and involves administering the anti-PSMA antibody to the mammal under conditions that permit binding of the anti-PSMA antibody to PSMA of any of the cells or portions thereof in the biological sample. Again, such administration can be carried out by any suitable method known to one of ordinary skill in the art.

In addition, the anti-PSMA antibodies of the present invention can be used in immunofluorescence techniques to examine human tissue, cell and bodily fluid specimens. In a typical protocol, slides containing cryostat sections of frozen, unfixed tissue biopsy samples or cytological smears are air dried, formalin or acetone fixed, and incubated with the monoclonal antibody preparation in a humidified chamber at room temperature. The slides are then washed and further incubated with a preparation of a secondary antibody directed against the monoclonal antibody, usually some type of anti-mouse immunoglobulin if the monoclonal antibodies used are derived from the fusion of a mouse spleen lymphocyte and a mouse myeloma cell line. This secondary antibody is tagged with a compound, for instance rhodamine or fluorescein isothiocyanate, that fluoresces at a particular wavelength. The staining pattern and intensities within the sample are then determined by fluorescent light microscopy and optionally photographically recorded.

As yet another alternative, computer enhanced fluorescence image analysis or flow cytometry can be used to examine tissue specimens or exfoliated cells, i.e., single cell preparations from aspiration biopsies of tumors using the anti-PSMA antibodies of this invention. The anti-PSMA antibodies of the invention are particularly useful in quantitation of live tumor cells, i.e., single cell preparations from aspiration biopsies of prostate tumors by computer enhanced fluorescence image analyzer or with a flow cytometer. The antibodies of the invention are particularly useful in such assays to differentiate benign from malignant prostate tumors since the PSMA protein to which the anti-PSMA antibodies bind is expressed in increased amounts by malignant tumors as compared to benign prostate tumors. The percent PSMA positive cell population, alone or in conjunction with determination of other attributes of the cells (e.g., DNA ploidy of these cells), may, additionally, provide very useful prognostic information by providing an early indicator of disease progression.

In yet another alternative embodiment, the antibodies of the present invention can be used in combination with other known antibodies to provide additional information regarding the malignant phenotype of a cancer.

The method of the present invention can be used to screen patients for diseases associated with the presence of cancerous cells or portions thereof. Alternatively, it can be used to identify the recurrence of such diseases, particularly when the disease is localized in a particular biological material of the patient. For example, recurrence of prostatic disease in the prostatic fossa may be encountered following radical prostatectomy. Using the method of the present invention, this recurrence can be detected by administering a short range radiolabeled antibody to the mammal and then detecting the label rectally, such as with a transrectal detector probe.

Alternatively, the contacting step can be carried out in a sample of serum or urine or other body fluids, including but not limited to seminal fluid, prostatic fluid, ejaculate, and the like, such as to detect the presence of PSMA in the body fluid. When the contacting is carried out in a serum or urine sample, it is preferred that the biological agent recognize substantially no antigens circulating in the blood other than PSMA. Since intact cells do not excrete or secrete PSMA into the extracellular environment, detecting PSMA in serum, urine, or other body fluids generally indicates that cells are being lysed or shed. Thus, the biological agents and methods of the present invention can be used to determine the effectiveness of a cancer treatment protocol by monitoring the level of PSMA in serum, urine or other body fluids.

In a particularly preferred embodiment of the method of detecting cancerous cells in accordance with the present invention, the anti-PSMA antibodies or an antigen-binding fragment thereof, binds to and is internalized with the prostate specific membrane antigen of such cells. Again, the biological agent is bound to a label effective to permit detection of the cells or portions thereof upon binding of the biological agent to and internalization of the biological agent with the prostate specific membrane antigen.

Biological agents suitable for detecting cancerous cells include anti-PSMA antibodies, such as monoclonal or polyclonal antibodies. In addition, antibody fragments, half-antibodies, hybrid derivatives, probes, and other molecular constructs may be utilized. These biological agents, such as antibodies, antigen-binding fragments thereof, probes, or ligands, bind to extracellular domains of prostate specific membrane antigens or portions thereof in cancerous cells. As a result, the biological agents bind not only to cells which are fixed or cells whose intracellular antigenic domains are otherwise exposed to the extracellular environment. Consequently, binding of the biological agents is concentrated in areas where there are prostate cells, irrespective of whether these cells are fixed or unfixed, viable or necrotic. Additionally or alternatively, these biological agents bind to and are internalized with prostate specific membrane antigens or portions thereof in normal, benign hyperplastic, and to a greater degree in cancerous cells.

The PSMA multimers and antibodies or antigen-binding fragments thereof can also be utilized in in vivo therapy of cancer. The PSMA multimers and antibodies or antigen-binding fragments thereof can be used with a compound which kills and/or inhibits proliferation of malignant cells or tissues. For instance, the antibodies can be covalently attached, either directly or via linker, to such a compound following administration and localization of the conjugates. When the antibody is used by itself, it may mediate tumor destruction by complement fixation or antibody-dependent cellular cytotoxicity. Alternatively, the PSMA multimer or antibody may be administered in combination with a chemotherapeutic drug to result in synergistic therapeutic effects (Baslya and Mendelsohn, 1994 Breast Cancer Res. and Treatment 29:127-138). A variety of different types of substances can be directly conjugated for therapeutic uses, including radioactive metal and non-metal isotopes, chemotherapeutic drugs, toxins, etc. as described above and known in the art (see, e.g., Vitetta and Uhr, 1985, Annu. Rev. Immunol. 3:197).

The antibodies or antigen-binding fragments thereof of the invention can also be administered together with complement. Accordingly, within the scope of the invention are compositions comprising antibodies or antigen-binding fragments thereof and serum or complement. These compositions are advantageous in that the complement is located in close proximity to the human antibodies or antigen-binding fragments thereof. Alternatively, the antibodies or antigen-binding fragments thereof of the invention and the complement or serum can be administered separately.

The PSMA multimers or antibodies can be administered with one or more immunostimulatory agents to induce or enhance an immune response, such as IL-2 and immunostimulatory oligonucleotides (e.g., those containing CpG motifs). Preferred immunostimulatory agents stimulate specific arms of the immune system, such as natural killer (NK) cells that mediate antibody-dependent cell cytotoxicity (ADCC).

Antigens, such as the PSMA dimers described herein, can be administered with one or more adjuvants to induce or enhance an immune response. An adjuvant is a substance which potentiates the immune response. Adjuvants of many kinds are well known in the art. Specific examples of adjuvants include monophosphoryl lipid A (MPL, SmithKline Beecham); saponins including QS21 (SmithKline Beecham); immunostimulatory oligonucleotides (e.g., CpG oligonucleotides described by Kreig et al., Nature 374:546-9, 1995); incomplete Freund's adjuvant; complete Freund's adjuvant; montanide; vitamin E and various water-in-oil emulsions prepared from biodegradable oils such as squalene and/or tocopherol, Quil A, Ribi Detox, CRL-1005, L-121, and combinations thereof.

Other agents which stimulate the immune response of the subject to PSMA multimer antigens can also be administered to the subject. For example, cytokines are also useful in vaccination protocols as a result of their lymphocyte regulatory properties. Many cytokines useful for such purposes will be known to one of ordinary skill in the art, including interleukin-2 (IL-2); IL-4; IL-5; IL-12, which has been shown to enhance the protective effects of vaccines (see, e.g., Science 268: 1432-1434, 1995); GM-CSF; IL-15; IL-18; combinations thereof, and the like. Thus cytokines can be administered in conjunction with antibodies, antigens, chemokines and/or adjuvants to increase an immune response.

Chemokines useful in increasing immune responses include but are not limited to SLC, ELC, MIP3α, MIP3β, IP-10, MIG, and combinations thereof.

The PSMA multimers or antibodies or antigen-binding fragments thereof of the present invention can be used in conjunction with other therapeutic treatment modalities. Such other treatments include surgery, radiation, cryosurgery, thermotherapy, hormone treatment, chemotherapy, vaccines, and other immunotherapies.

Also encompassed by the present invention is a method which involves using the PSMA multimers or antibodies or antigen-binding fragments thereof for prophylaxis. For example, these materials can be used to prevent or delay development or progression of cancer.

Use of the cancer therapy of the present invention has a number of benefits. Since the anti-PSMA antibodies or antigen-binding fragments thereof according to the present invention preferentially target prostate cancer cells, other tissue is spared. As a result, treatment with such biological agents is safer, particularly for elderly patients. Treatment according to the present invention is expected to be particularly effective, because it directs high levels of anti-PSMA antibodies or antigen-binding fragments thereof to the bone marrow and lymph nodes where prostate cancer metastases predominate. Moreover, tumor sites for prostate cancer tend to be small in size and, therefore, easily destroyed by cytotoxic agents. Treatment in accordance with the present invention can be effectively monitored with clinical parameters such as serum prostate specific antigen and/or pathological features of a patient's cancer, including stage, Gleason score, extracapsular, seminal, vesicle or perineural invasion, positive margins, involved lymph nodes, etc. Alternatively, these parameters can be used to indicate when such treatment should be employed.

Because the antibodies or antigen-binding fragments thereof of the present invention bind to living cells, therapeutic methods using these biological agents are much more effective than those which target lysed cells. For the same reasons, diagnostic and imaging methods which determine the location of living normal, benign hyperplastic, or cancerous cells are much improved by employing the antibodies or antigen-binding fragments thereof of the present invention. In addition, the ability to differentiate between living and dead cells can be advantageous, especially to monitor the effectiveness of a particular treatment regimen.

Also within the scope of the invention are kits comprising the compositions of the invention and instructions for use. The kits can further contain at least one additional reagent, such as complement, or one or more additional antibodies of the invention (e.g., an antibody having a complementary activity which binds to an epitope in PSMA antigen distinct from the first antibody). Other kits can include the PSMA multimers described herein below.

The kits provided herein include any of the compositions described and instructions for the use of these compositions. The instructions can include instructions for mixing a particular amount of a diluent with a particular amount of a PSMA dimeric composition, whereby a final formulation for injection or infusion is prepared. Therefore, kits are also provided, which include the compositions of the invention and an adjuvant (e.g., alum) or diluent and instructions for mixing. Kits are also provided wherein the compositions of the inventions are provided in a vial or ampoule with a septum or a syringe. Other kits where the composition is in lyophilized form are also provided. The instructions, therefore, would take a variety of forms depending on the presence or absence of diluent or other agents (e.g., therapeutic agents). The instructions can include instructions for treating a patient with an effective amount of dimeric PSMA. It also will be understood that the containers containing the pharmaceutical preparation, whether the container is a bottle, a vial with a septum, an ampoule with a septum, an infusion bag, and the like, can contain indicia such as conventional markings which change color when the pharmaceutical preparation has been autoclaved or otherwise sterilized.

Kits containing the antibodies or antigen-binding fragments thereof of the invention can be prepared for in vitro diagnosis, prognosis and/or monitoring cancer by the immunohistological, immunocytological and immunoserological methods described above. The components of the kits can be packaged either in aqueous medium or in lyophilized form. When the antibodies or antigen-binding fragments thereof are used in the kits in the form of conjugates in which a label moiety is attached, such as an enzyme or a radioactive metal ion, the components of such conjugates can be supplied either in fully conjugated form, in the form of intermediates or as separate moieties to be conjugated by the user or the kit.

A kit may comprise a carrier being compartmentalized to receive in close confinement therein one or more container means or series of container means such as test tubes, vials, flasks, bottles, syringes, or the like. A first of said container means or series of container means may contain one or more anti-PSMA antibodies or antigen-binding fragments thereof or PSMA. A second container means or series of container means may contain a label or linker-label intermediate capable of binding to the primary anti-PSMA antibodies (or fragment thereof.

It should be understood that the pharmaceutical preparations of the invention will typically be held in bottles, vials, ampoules, infusion bags, and the like, any one of which may be sparged to eliminate oxygen or purged with nitrogen. In some embodiments, the bottles vials and ampoules are opaque, such as when amber in color. Such sparging and purging protocols are well known to those of ordinary skill in the art and should contribute to maintaining the stability of the pharmaceutical preparations. The pharmaceutical preparations also, in certain embodiments, are expected to be contained within syringes.

Kits for use in in vivo tumor localization and therapy method containing the anti-PSMA antibodies or antigen-binding fragments thereof conjugated to other compounds or substances can be prepared. The components of the kits can be packaged either in aqueous medium or in lyophilized form. When the antibodies or antigen-binding fragments thereof are used in the kits in the form of conjugates in which a label or a therapeutic moiety is attached, such as a radioactive metal ion or a therapeutic drug moiety, the components of such conjugates can be supplied either in fully conjugated form, in the form of intermediates or as separate moieties to be conjugated by the user of the kit.

In one aspect of the invention, a method for modulating at least one enzymatic activity of PSMA, the activity selected from the group consisting of N-acetylated α-linked acidic dipeptidase (NAALADase), folate hydrolase, dipeptidyl dipeptidase IV and -γ-glutamyl hydrolase activity or combination thereof in vitro or in vivo. The modulation may be enhancement or inhibition of at least one enzymatic activity of PSMA.

In a preferred embodiment, the invention provides methods for inhibiting at least one enzymatic activity of PSMA, the activity selected from the group consisting of N-acetylated α-linked acidic dipeptidase (NAALADase), folate hydrolase, dipeptidyl dipeptidase IV and γ-glutamyl hydrolase activity or combination thereof in vitro or in vivo. The method comprises contacting a NAALADase, a folate hydrolase, a dipeptidyl dipeptidase IV and/or a γ-glutamyl hydrolase with an amount of an isolated antibody or antigen-binding fragment thereof of the invention under conditions wherein the isolated monoclonal antibody or antigen-binding fragment thereof inhibits NAALADase, folate hydrolase, dipeptidyl dipeptidase IV or γ-glutamyl hydrolase activity.

Tissue levels of NAALADase can be determined by detergent solubilizing homogenizing tissues, pelleting the insoluble material by centrifugation and measuring the NAALADase activity in the remaining supernatant. Likewise, the NAALADase activity in bodily fluids can also be measured by first pelleting the cellular material by centrifugation and performing a typical enzyme assay for NAALADase activity on the supernatant. NAALADase enzyme assays have been described by Frieden, 1959, J. Biol, Chem., 234:2891. In this assay, the reaction product of the NAALADase enzyme is glutamic acid. This is derived from the enzyme catalyzed cleavage of N-acetylaspartylglutamate to yield N-acetylaspartic acid and glutamic acid. Glutamic acid, in a NAD(P)⁺ requiring step, yields 2-oxoglutarate plus NAD(P)H in a reaction catalyzed by glutamate dehydrogenase. Progress of the reaction can easily and conveniently be measured by the change in absorbance at 340 nm due to the conversion of NAD(P)⁺ to NAD(P)H.

Folate hydrolase activity of PSMA can be measured by performing enzyme assays as described by Heston and others (e.g., Clin. Cancer Res. 2(9):1445-51, 1996; Urology 49(3A Suppl):104-12,1997). Folate hydrolases such as PSMA remove the gamma-linked glutamates from polyglutamated folates. Folate hydrolase activity can be measured using substrates such as methotrexate tri-gamma glutamate (MTXGlu3), methotrexate di-gamma glutamate (MTXGlu2) and pteroylpentaglutamate (PteGlu5), for example using capillary electrophoresis (see Clin. Cancer Res. 2(9):1445-51, 1996). Timed incubations of PSMA with polyglutamated substrates is followed by separation and detection of hydrolysis products.

The invention also includes isolated antibodies and binding fragments thereof that selectively bind PSMA multimers. As used herein, particularly with respect to the binding of PSMA multimers by the anti-PSMA antibodies and binding fragments, “selectively binds” means that an antibody preferentially binds to a PSMA protein multimer (e.g., with greater avidity, greater binding affinity) rather than to a PSMA protein monomer. In preferred embodiments, the antibodies of the invention bind to a PSMA protein multimer with an avidity and/or binding affinity that is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 7-fold, 10-fold, 20-fold, 30-fold, 40-fold, 50-fold, 70-fold, 100-fold, 200-fold, 300-fold, 500-fold, 1000-fold or more than that exhibited by the antibody for a PSMA protein monomer. Preferably, the antibody selectively binds a PSMA protein multimer, and not a PSMA protein monomer, i.e., substantially exclusively binds to a PSMA protein multimer. Most preferably, the antibody selectively binds a PSMA protein dimer.

The isolated antibody or binding fragment that selectively binds a PSMA protein multimer can, in some embodiments, modulate enzymatic activity of the PSMA protein multimer. In one such embodiment, the antibody inhibits at least one enzymatic activity such as NAALADase activity, folate hydrolase activity, dipeptidyl dipeptidase IV activity, γ-glutamyl hydrolase activity, or combinations thereof. In another embodiment, the antibody enhances at least one enzymatic activity such as NAALADase activity, folate hydrolase activity, dipeptidyl dipeptidase IV activity, γ-glutamyl hydrolase activity, or combinations thereof.

A PSMA protein multimer, as used herein, is a protein complex of at least two PSMA proteins or fragments thereof. The PSMA protein multimers can be composed of various combinations of full-length PSMA proteins (e.g., SEQ ID NO: 1), recombinant soluble PSMA (rsPSMA, e.g., amino acids 44-750 of SEQ ID NO: 1) and fragments of the foregoing that form multimers (i.e., that retain the protein domain required for forming dimers and/or higher order multimers of PSMA). In preferred embodiments, at least one of the PSMA proteins forming the multimer is a recombinant, soluble PSMA (rsPSMA) polypeptide. Preferred PSMA protein multimers are dimers, particularly those formed from recombinant soluble PSMA protein. A particularly preferred embodiment is a rsPSMA homodimer.

The PSMA protein multimers referred to herein are believed to assume a native conformation and preferably have such a conformation. The PSMA proteins in certain embodiments are noncovalently bound together to form the PSMA protein multimer. For example, it has been discovered that PSMA protein noncovalently associates to form dimers under non-denaturing conditions, as described in the Examples below.

The PSMA protein multimers can, and preferably do, retain the activities of PSMA. The PSMA activity may be an enzymatic activity, such as folate hydrolase activity, NAALADase activity, dipeptidyl peptidase IV activity and γ-glutamyl hydrolase activity. Methods for testing the PSMA activity of multimers are well known in the art (reviewed by O'Keefe et al. in: Prostate Cancer: Biology, Genetics, and the New Therapeutics, L. W. K. Chung, W. B. Isaacs and J. W. Simons (eds.) Humana Press, Totowa, N.J., 2000, pp. 307-326), some of which are described in the Examples herein below.

In certain aspects, the invention also includes compositions including one or more of the isolated PSMA protein multimers described herein, such as the PSMA protein dimer. In preferred embodiments, a PSMA protein multimer composition contains at least about 10% PSMA protein multimer. In other embodiments, the PSMA protein multimer composition contains at least about 20%, 30%, 40%, 50%, 60%, 70%, 75%, 80%, 85%, 90%, 95%, 99% or 99.5% PSMA protein multimer. In a preferred embodiment, the PSMA protein multimer composition contains substantially pure PSMA protein multimer, with substantially no PSMA protein monomer. It is understood that the list of specific percentages includes by inference all of the unnamed percentages between the recited percentages.

As used herein with respect to polypeptides, proteins or fragments thereof, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

Fragments of a PSMA protein preferably are those fragments which retain a distinct functional capability of the PSMA protein. Functional capabilities which can be retained in a fragment include binding of other PSMA molecules to form dimers and higher order multimers, interaction with antibodies, interaction with other polypeptides or fragments thereof, and enzymatic activity. Other PSMA protein fragments, e.g., other recombinant soluble fragments of SEQ ID NO: 1, can be selected according to their functional properties. For example, one of ordinary skill in the art can prepare PSMA fragments recombinantly and test those fragments according to the methods exemplified below.

Modifications to a PSMA polypeptide are typically made to the nucleic acid which encodes the PSMA polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the PSMA amino acid sequence.

In general, modified PSMA polypeptides include polypeptides which are modified specifically to alter a feature of the polypeptide unrelated to its physiological activity. For example, cysteine residues can be substituted or deleted to prevent unwanted disulfide linkages. Similarly, certain amino acids can be changed to enhance expression of a PSMA polypeptide by eliminating proteolysis by proteases in an expression system (e.g., dibasic amino acid residues in yeast expression systems in which KEX2 protease activity is present).

Modifications conveniently are prepared by altering a nucleic acid molecule that encodes the PSMA polypeptide. Mutations of a nucleic acid which encode a PSMA polypeptide preferably preserve the amino acid reading frame of the coding sequence, and preferably do not create regions in the nucleic acid which are likely to hybridize to form secondary structures, such a hairpins or loops, which can be deleterious to expression of the modified polypeptide.

Modifications can be made by selecting an amino acid substitution, or by random mutagenesis of a selected site in a nucleic acid which encodes the PSMA polypeptide. Modified PSMA polypeptides then can be expressed and tested for one or more activities (e.g., antibody binding, enzymatic activity, multimeric stability) to determine which mutation provides a modified polypeptide with the desired properties. Further mutations can be made to modified PSMA polypeptides (or to non-modified PSMA polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a PSMA coding sequence or cDNA clone to enhance expression of the polypeptide. The activity of modified PSMA polypeptides can be tested by cloning the gene encoding the modified PSMA polypeptide into a bacterial or mammalian expression vector, introducing the vector into an appropriate host cell, expressing the modified PSMA polypeptide, and testing for a functional capability of the PSMA polypeptides as disclosed herein. The foregoing procedures are well known to one of ordinary skill in the art.

The skilled artisan will also realize that conservative amino acid substitutions may be made in PSMA polypeptides to provide functionally equivalent PSMA polypeptides, i.e., modified PSMA polypeptides that retain the functional capabilities of PSMA polypeptides. These functionally equivalent PSMA polypeptides include those PSMA polypeptides or proteins that are capable of associating to form multimers, particularly dimers. As used herein, a “conservative amino acid substitution” refers to an amino acid substitution which does not alter the relative charge or size characteristics of the protein in which the amino acid substitution is made. Modified PSMA polypeptides can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent PSMA polypeptides include conservative amino acid substitutions of SEQ ID NO: 1, or fragments thereof, such as the recombinant soluble PSMA polypeptide (amino acids 44-750 of SEQ ID NO: 1). Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in PSMA polypeptides typically are made by alteration of a nucleic acid encoding a PSMA polypeptide. Conservatively substituted PSMA polypeptides include those with 1, 2, 3, 4, 5, 6, 7, 8, 9, 10, 15, 20, or more substitutions. Such substitutions can be made by a variety of methods known to one of ordinary skill in the art. For example, amino acid substitutions may be made by PCR-directed mutation, site-directed mutagenesis, or by chemical synthesis of a gene encoding a PSMA polypeptide. Where amino acid substitutions are made to a small fragment of a PSMA polypeptide, the substitutions can be made by directly synthesizing the peptide. The activity of functionally equivalent fragments of PSMA polypeptides can be tested by cloning the gene encoding the altered PSMA polypeptide into a bacterial or mammalian expression vector, introducing the vector into an appropriate host cell, expressing the altered PSMA polypeptide, and testing for a functional capability of the PSMA polypeptides as disclosed herein.

The PSMA protein multimers as described herein have a number of uses, some of which are described elsewhere herein. The multimers are useful for testing of compounds that modulate PSMA enzymatic activity or PSMA multimerization. The multimers can be used to isolate antibodies that selectively bind PSMA, including those selective for conformational epitopes, those selective for binding PSMA multimers and not PSMA monomers, and those that selectively modulate an enzymatic activity of PSMA. The multimers, particularly dimeric PSMA, also can be used to induce or increase immune responses to PSMA, as vaccine compositions.

Agents that selectively modulate an enzymatic activity of PSMA include agents that inhibit or enhance at least one enzymatic activity of PSMA, such as NAALADase activity, folate hydrolase activity, dipeptidyl dipeptidase IV activity, γ-glutamyl hydrolase activity, or combinations thereof.

Thus methods of screening for candidate agents that modulate at least one enzymatic activity of a PSMA enzyme are provided in accordance with the invention. The methods can include mixing the candidate agent with an isolated PSMA protein multimer to form a reaction mixture, thereby contacting the PSMA enzyme with the candidate agent. The methods also include adding a substrate for the PSMA enzyme to the reaction mixture, and determining the amount of a product formed from the substrate by the PSMA enzyme. Such methods are adaptable to automated, high-throughput screening of compounds. A decrease in the amount of product formed in comparison to a control is indicative of an agent capable of inhibiting at least one enzymatic activity of the PSMA enzyme. An increase in the amount of product formed in comparison to a control is indicative of an agent capable of enhancing at least one enzymatic activity of the PSMA enzyme. The PSMA enzyme can be NAALADase, folate hydrolase, dipeptidyl dipeptidase IV and/or γ-glutamyl hydrolase. The PSMA enzyme preferably is a PSMA multimer that includes recombinant soluble PSMA, most preferably a noncovalently associated dimer of PSMA in a native conformation.

The reaction mixture comprises a candidate agent. The candidate agent is preferably an antibody, a small organic compound, or a peptide, and accordingly can be selected from combinatorial antibody libraries, combinatorial protein libraries, or small organic molecule libraries. Typically, a plurality of reaction mixtures are run in parallel with different agent concentrations to obtain a different response to the various concentrations. Typically, one of these concentrations serves as a negative control, i.e., at zero concentration of agent or at a concentration of agent below the limits of assay detection.

Candidate agents encompass numerous chemical classes, although typically they are organic compounds, proteins or antibodies (and fragments thereof that bind antigen). In some preferred embodiments, the candidate agents are small organic compounds, i.e., those having a molecular weight of more than 50 yet less than about 2500, preferably less than about 1000 and, more preferably, less than about 500. Candidate agents comprise functional chemical groups necessary for structural interactions with polypeptides and/or nucleic acids, and typically include at least an amine, carbonyl, hydroxyl, or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random or non-random peptides, combinatorial libraries of proteins or antibodies, and the like. Alternatively, libraries of natural compounds in the form of bacterial, fungal, plant, and animal extracts are available or readily produced. Additionally, natural and synthetically produced libraries and compounds can be readily be modified through conventional chemical, physical, and biochemical means. Further, known agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-agent binding. Such a reagent may also reduce non-specific or background interactions of the reaction components. Other reagents that improve the efficiency of the assay such as protease inhibitors, nuclease inhibitors, antimicrobial agents, and the like may also be used.

The mixture of the foregoing reaction materials is incubated under conditions whereby, the candidate agent interacts with the PSMA enzyme. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of PSMA enzyme activity is detected by any convenient method available to the user. For example, the reaction mixture can contain a substrate for the PSMA enzyme. Preferably the substrate and/or the product formed by the action of the PSMA enzyme are detectable. The substrate usually comprises, or is coupled to, a detectable label. A wide variety of labels can be used, such as those that provide direct detection (e.g., radioactivity, luminescence, optical, or electron density, etc) or indirect detection (e.g., epitope tag such as the FLAG epitope, enzyme tag such as horseradish peroxidase, etc.). The label may be bound to the substrate, or incorporated into the structure of the substrate.

A variety of methods may be used to detect the label, depending on the nature of the label and other assay components. For example, the label may be detected while bound to the substrate or subsequent to separation from the substrate. Labels may be directly detected through optical or electron density, radioactive emissions, nonradiative energy transfers, etc. or indirectly detected with antibody conjugates, strepavidin-biotin conjugates, etc. Methods for detecting a variety of labels are well known in the art.

### EXAMPLES

Materials and Methods

DNA Constructs. All secreted PSMA constructs were derived from the original human PSMA clone p55A provided by Dr. W. D. W. Heston (Israeli et al., Cancer Res. 53: 227-230, 1993). The constructs were subcloned into expression vector PPI4 (Trkola et al., Nature 384: 184-187, 1996) for high-level expression and secretion in mammalian cells. Recombinant soluble PSMA (rsPSMA) corresponds to the entire extracellular domain of PSMA (amino acids 44-750 of SEQ ID NO:1 (GenBank Protein Accession number AAA60209)).

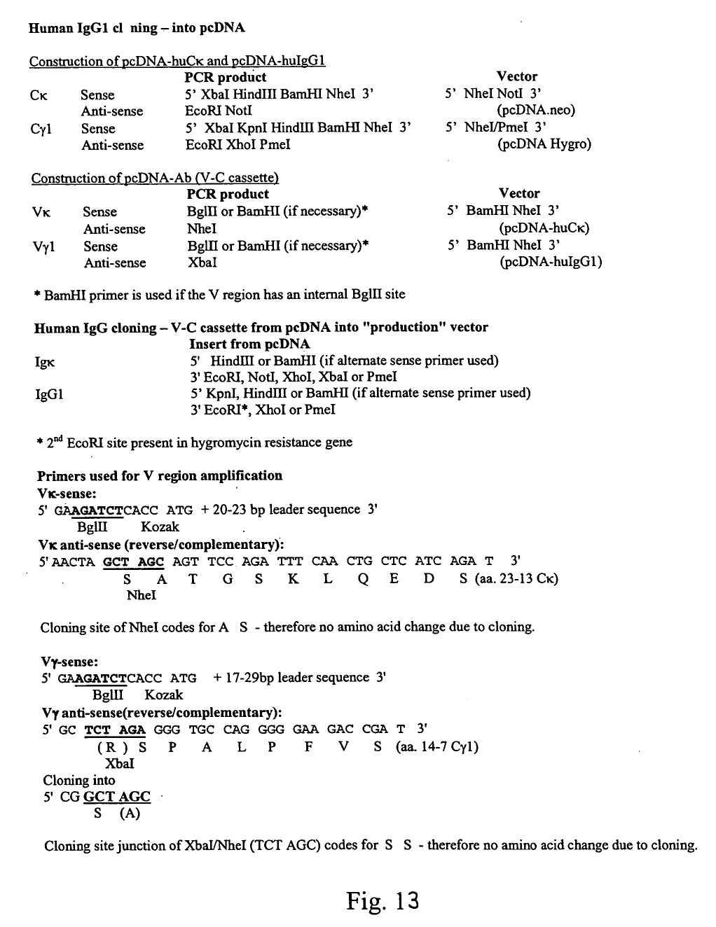

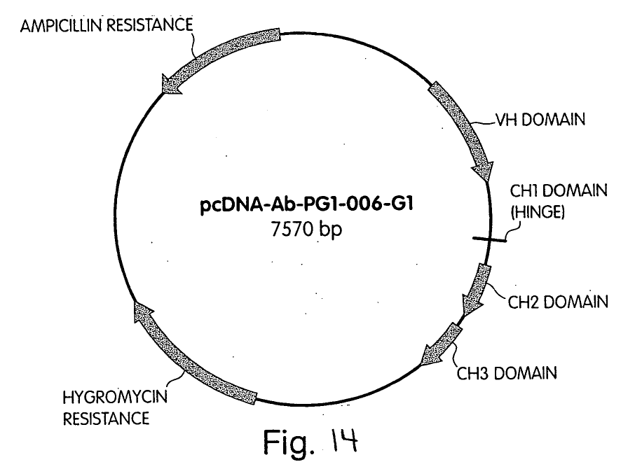

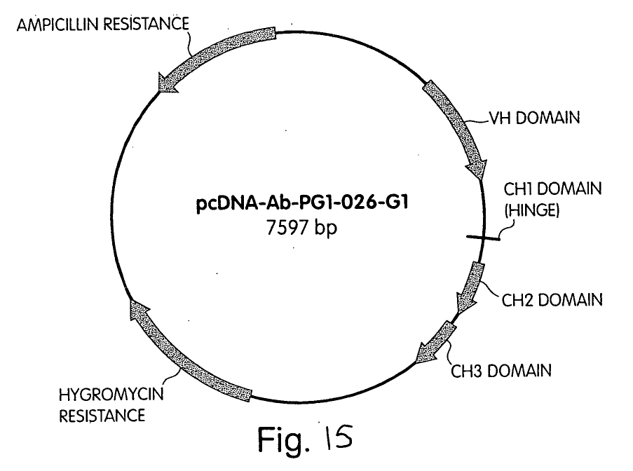

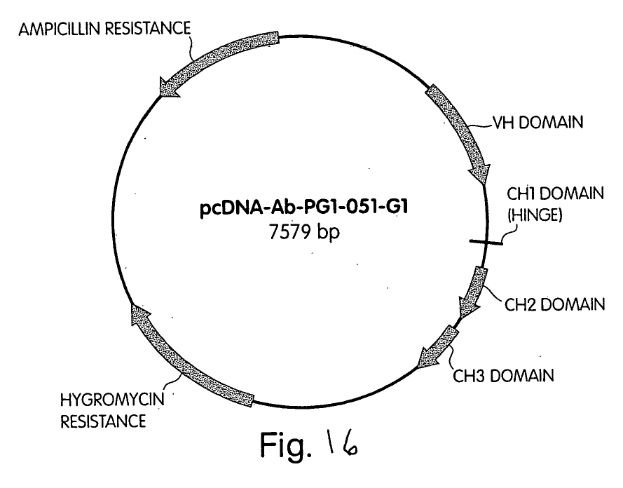

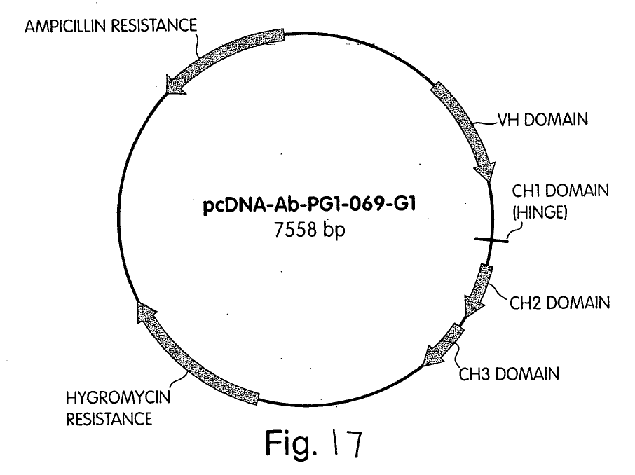

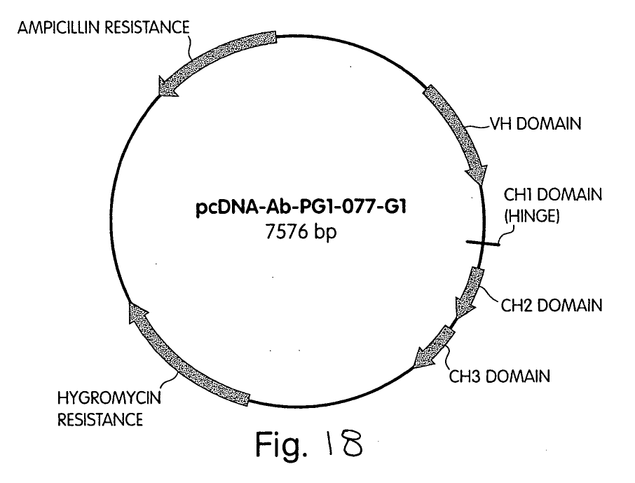

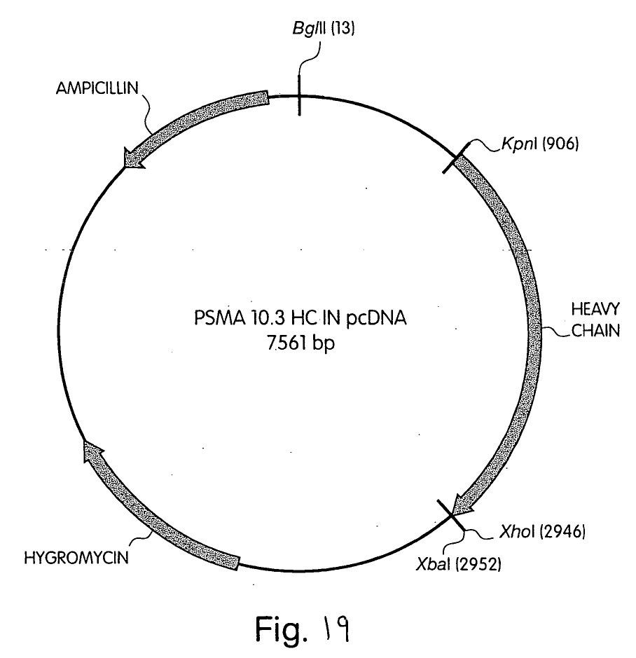

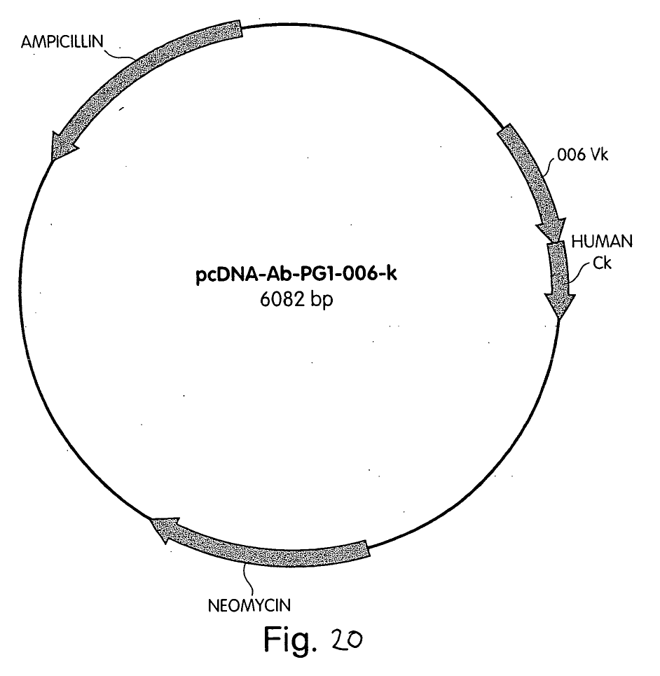

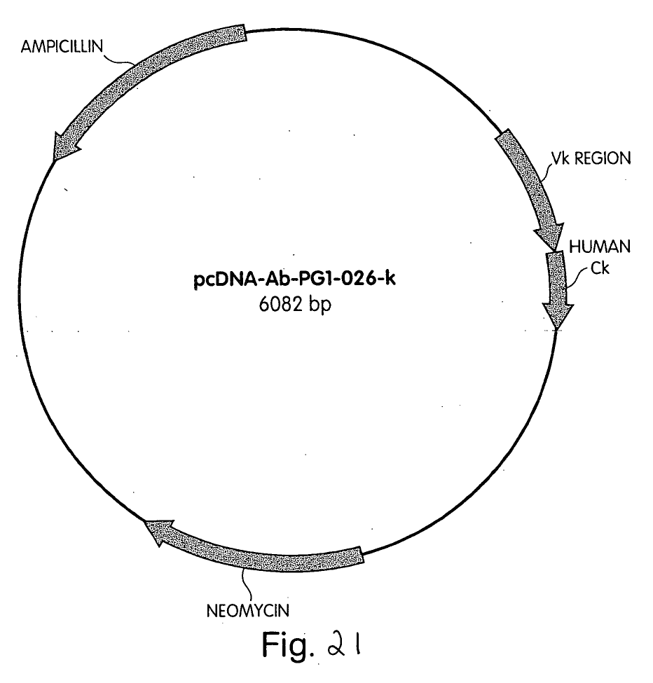

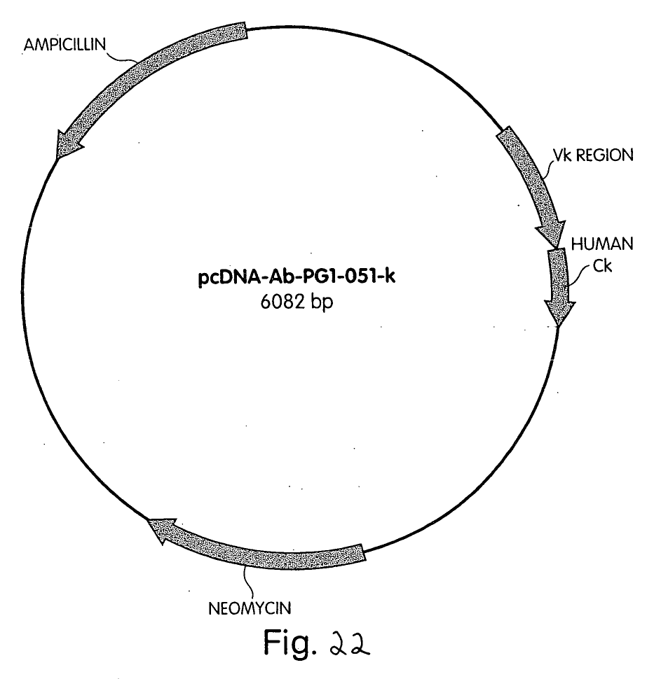

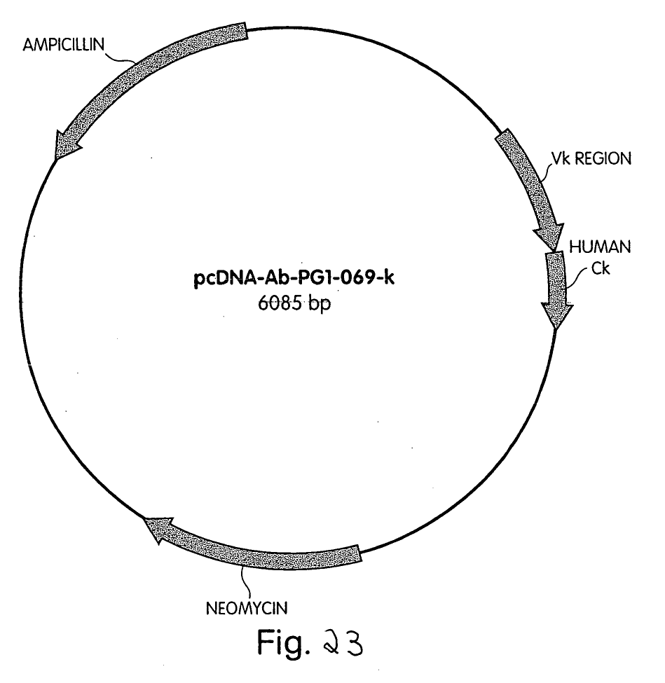

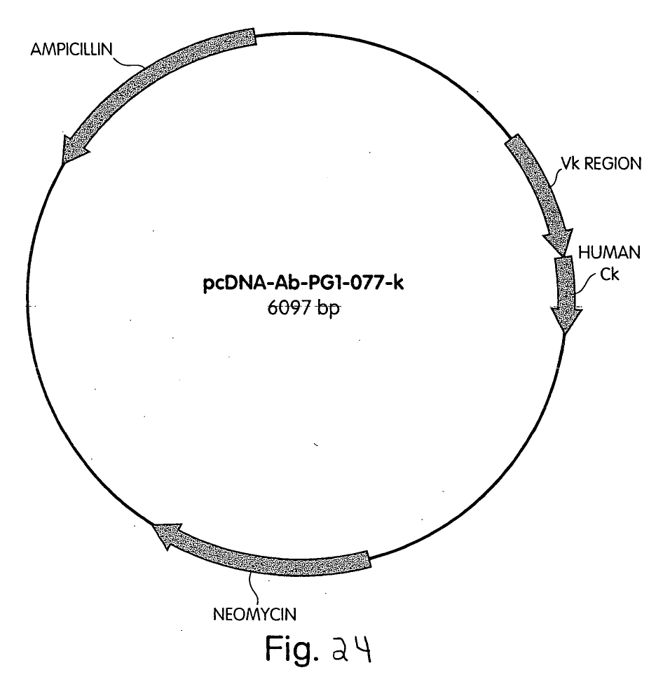

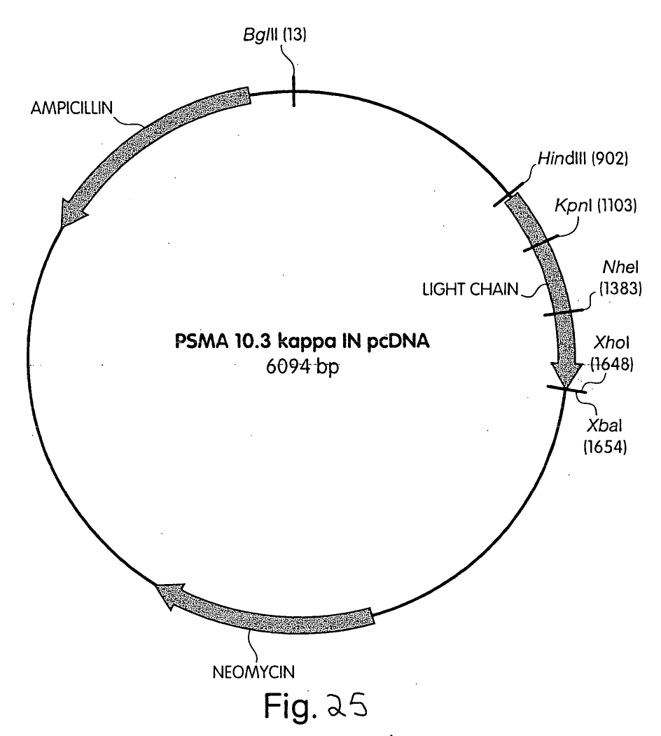

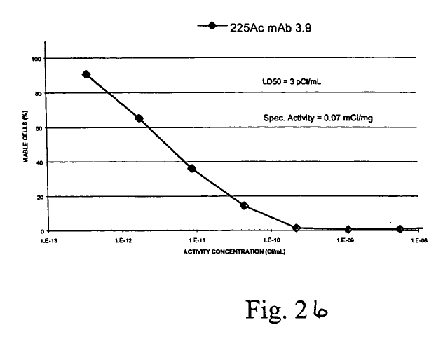

pcDNA Plasmid Constructs: Nucleic acid molecules encoding the anti-PSMA antibodies 10.3, 006, 026, 051, 069 and 077 were cloned into plasmid pcDNA. The cloning protocol is given in FIG. 13. Primers (SEQ ID NOs: 33-36, sense and anti-sense) used for the variable region amplifications are also shown. The plasmids constructed for anti-PSMA antibodies 006, 026, 051, 069, 077 and 10.3 contain nucleotide sequences encoding the heavy chain of the antibodies (SEQ ID NOs: 2-7; PTA-4403, PTA-4405, PTA-4407, PTA-4409, PTA-4411, PTA-4413, respectively) or contain nucleotide sequences encoding light chain of the antibodies (SEQ ID NOs: 8-13; PTA-4404, PTA-4406, PTA-4408, PTA-4410, PTA-4412 and PTA-4414, respectively). Plasmid maps are given in FIGS. 14-25.

Western Blots. Cells were lysed in PBS containing 1 mM EDTA, 1% NP-40, 1% Triton X-100, and 5 mg/ml aprotinin and cell debris was removed by centrifugation at 3000 g for 30 min at 4° C. Lysates were separated on a 5-20% gradient gel before transfer to nitrocellulose membranes. The resulting blots were blocked in PBS containing 5% milk, 0.02% SDS and 0.1% Triton X-100 before incubation with MAB544 primary antibody (Maine Biotechnologies) at a concentration of 2 mg/ml. After three washes, blots were incubated with a goat anti-mouse HRP-conjugated secondary antibody at a concentration of 0.2 mg/ml. Blots are visualized using the Renaissance chemiluminescence system (Perkin-Elmer Life Sciences, Boston, Mass.).

ELISA. Cells were lysed in PBS containing 1 mM EDTA, 1% NP-40, 1% Triton X-100, and 5 mg/ml aprotinin. The resulting cell membranes were plated onto 96-well plates and dried in a sterile hood overnight. The plates were then blocked with PBS containing casein and Tween-20 before addition of mouse sera or hybridoma supernatants, using purified MAB544 (Maine Biotechnologies) or 7E11 (Cytogen) as a standard. After washing in PBS, an alkaline phosphatase conjugated secondary antibody (subclass specific) was incubated and subsequently washed in PBS. The pNPP substrate was then added for colorimetric detection at a wavelength of 405 nm.

Flow Cytometry. Wild-type 3T3 or PSMA-expressing 3T3 cells (10⁶ cells per condition) were washed in PBS containing 0.1% NaN₃. Antibodies or sera were then added (1:100 dilution in PBS) and incubated on ice for 30 minutes. After washing in PBS+0.1% NaN₃, the cells were incubated with anti-mouse IgG+IgM (Calbiotech) for 30 minutes on ice. Cells were washed again in PB S+0.1% NaN₃ and analyzed by flow cytometry.

### Example 1

### Generation of a Panel of Monoclonal Antibodies (mAbs) to Conformational Epitopes on PSMA

A panel of anti-PSMA mAbs that represent promising candidates for therapy was created. Briefly, the mAbs were generated as follows: BALB/c mice were immunized subcutaneously with recombinant PSMA at approximately three-week intervals. After a total of 4 injections, mice were sacrificed and their splenocytes fused with a myeloma cell line using standard techniques in order to create hybridomas. Individual hybridoma supernatants were screened by ELISA for reactivity with PSMA derived from either LNCaP human prostate tumor cells or from 3T3 cells engineered to express full-length human PSMA (3T3-PSMA cells). Positive clones were secondarily screened by flow cytometry for specific reactivity with intact 3T3-PSMA and LNCaP cells so as to select antibodies that recognize native, cell-surface PSMA and thus have the greatest therapeutic potential.

Mice having the ability to produce human antibodies (XenoMouse, Abgenix; Mendez et al., Nature Genetics 15:146, 1997) were immunized subcutaneously once or twice weekly with 5×10⁶ LNCaP cells adjuvanted with alum or Titermax Gold (Sigma Chemical Co., St. Louis, Mo.). Animals were boosted twice with 10 μg of recombinant PSMA protein immunoaffinity captured onto protein G magnetic microbeads (Miltenyi Biotec, Auburn, Calif.). PSMA mAb 3.11 was used for capture. Splenocytes were fused with NSO myeloma cells and the hybridomas that resulted were screened as above by flow cytometry to detect clones producing antibodies reactive with the extracellular portion of PSMA. One clone, 10.3 (PTA-3347), produced such antibodies.

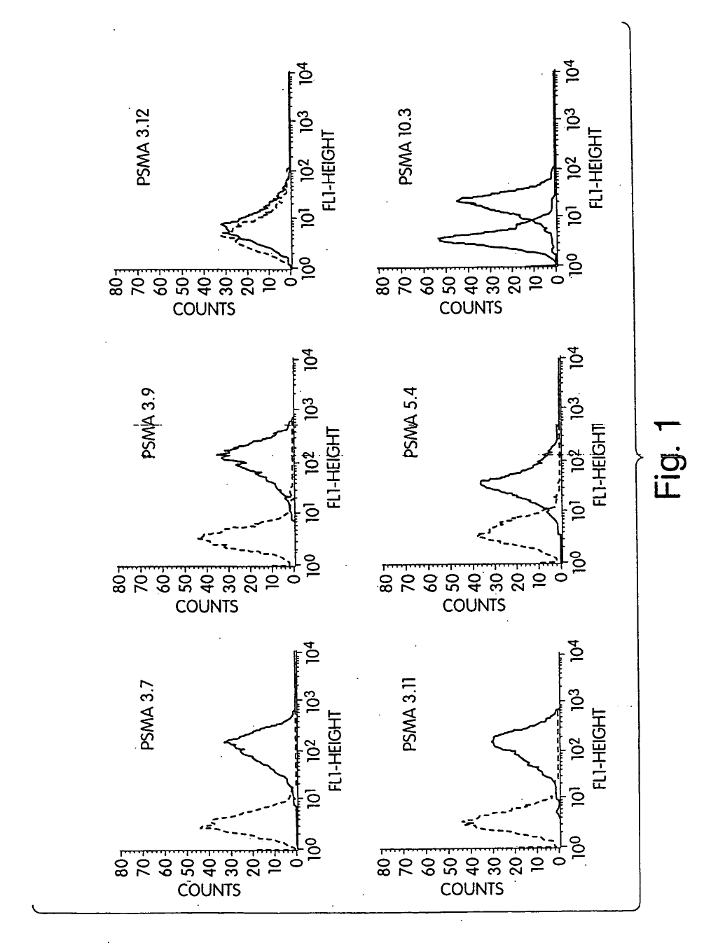

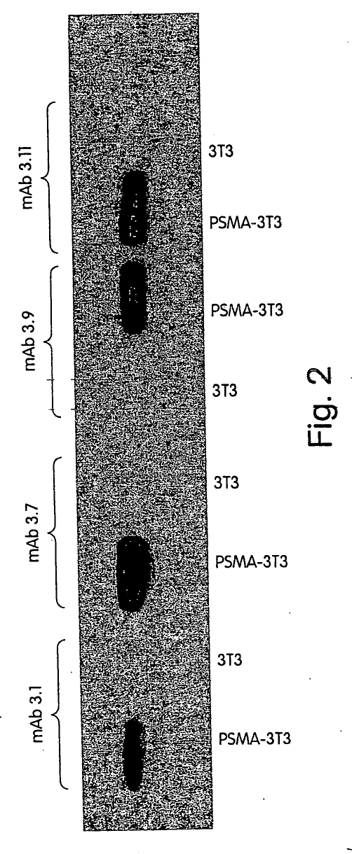

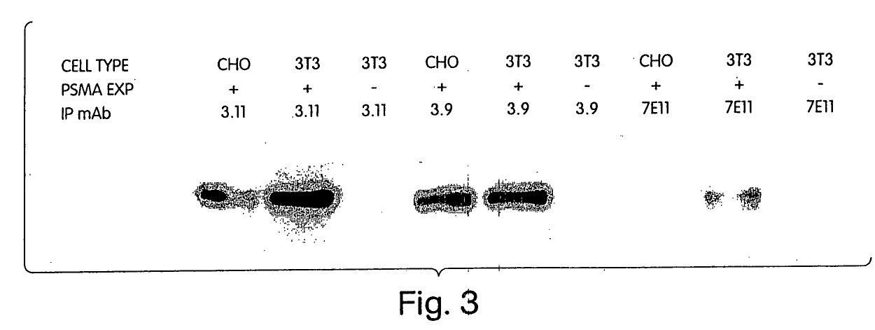

These methods have yielded a high proportion of mAbs that react exclusively with conformation-specific epitopes on cell-surface PSMA. As shown in FIG. 1, several (mAbs 3.7, 3.9, 3.11, 5.4, and 10.3) but not all (mAb 3.12) mAbs specifically bind viable PSMA-expressing cells. Using recombinant soluble PSMA proteins expressed in Chinese hamster ovary (CHO) cell lines, it further was demonstrated that the mAbs bind epitopes in the extracellular region of PSMA. The mAbs were also tested for their ability to immunoprecipitate native PSMA from 3T3-PSMA cell lysates. The mAbs positive in flow cytometry (FIG. 1) were also effective in immunoprecipitation (FIG. 2), whereas mAb 3.12 was unreactive. FIG. 3 shows the recognition of non-denatured fill-length PSMA and recombinant soluble PSMA by several PSMA antibodies that recognize PSMA conformation. This further confirms that these methods yield a preponderance of mAbs that efficiently recognize native PSMA.

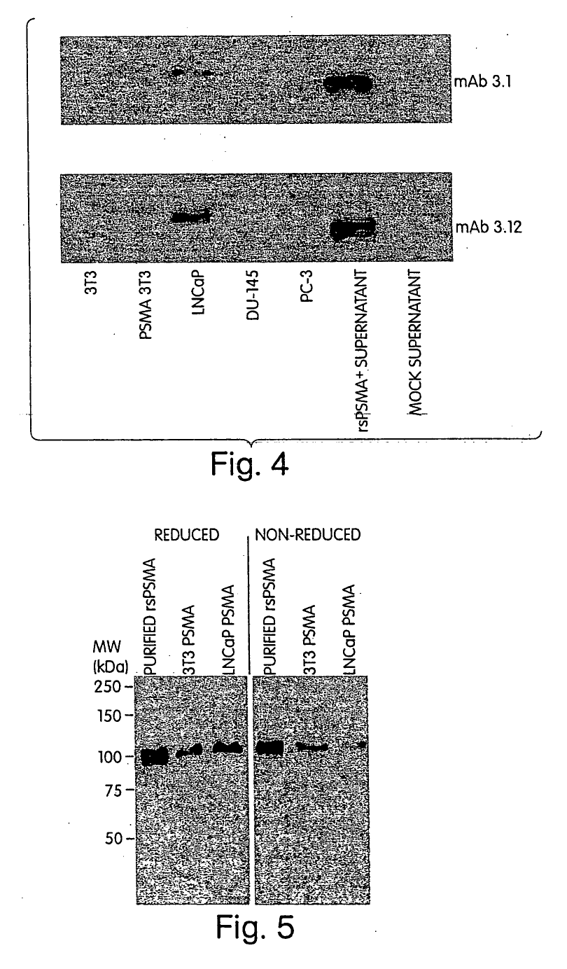

The mAbs were tested for reactivity with denatured PSMA by Western blot analysis (FIG. 4). Lysates from the indicated cells and samples (controls: 3T3 cells, PSMA-negative human prostate cell lines PC-3 and DU145, mock supernatant; PSMA-positive samples: PSMA-expressing 3T3 cells, PSMA-positive human prostate cell line LNCaP, rsPSMA-positive supernatant) were resolved by SDS-PAGE, electroblotted, and probed with anti-PSMA mAbs 3.1 and 3.12 (ATCC Patent Deposit Designations PTA-3639 and PTA-3640, respectively). Four mAbs tested in parallel (3.7, 3.8, 3.9, 3.11) showed no reactivity to either full-length or secreted rsPSMA proteins. 7E11 mAb immunoprecipitated full-length but not secreted rsPSMA.

The mAbs reactive in flow cytometry and immunoprecipitation (mAbs 3.7, 3.9, 3.11, 5.4, and 10.3) were all unreactive in Western blot analysis, indicating that the mAbs do not recognize linear epitopes. Taken together, the data strongly suggest that these 5 mAbs recognize conformation-specific epitopes located in the extracellular domain of PSMA. Since mAbs to conformational epitopes typically possess the greatest affinity and specificity for antigen, they represent preferred candidates for therapy.

The reactivities of certain anti-PSMA antibodies are described in Table 2:

TABLE 2
Anti-PSMA Antibody Properties

| | Reactivity | | | | |
|---|---|---|---|---|---|
| | | Flow | | | |
| mAb | ELISA | Cytometry | IP | Western | Epitope |
| 3.1 | + | + | + | + | Linear, Extracellular, |
| | | | | | exposed on native PSMA |
| 3.7 | + | + | + | − | Conformational, |
| | | | | | extracellular |
| 3.8 | + | + | + | − | Conformational, |
| | | | | | extracellular |
| 3.9 | + | + | + | − | Conformational, |
| | | | | | extracellular |
| 3.11 | + | + | + | − | Conformational, |
| | | | | | extracellular |
| 3.12 | + | − | − | + | Linear, Extracellular, |
| | | | | | not exposed on native |
| | | | | | PSMA |
| 5.4 | + | + | + | − | Conformational, |
| | | | | | extracellular |
| 7.1 | + | − | − | + | Linear, Extracellular, |
| | | | | | not exposed on native |
| | | | | | PSMA |
| 7.3 | + | + | + | − | Conformational, |
| | | | | | extracellular |
| 10.3 | + | + | + | − | Conformational, |
| | | | | | extracellular |
| 1.8.3 | + | + | | − | Extracellular |
| A3.1.3 | + | + | | − | Extracellular |
| A3.3.1 | + | + | | − | Extracellular |

The mAbs were determined by ELISA to be primarily of the mouse IgG2a, mouse IgG2b and human IgG1 isotypes, which mediate potent effector functions. Although a number of anti-PSMA mAbs have been described over the years and evaluated for therapeutic potential (see, e.g., Liu, H. et al. Cancer Res. 57: 3629-3634, 1997; Chang, S. S. et al. Cancer Res. 59: 3192-3198, 1999; Murphy, G. P. et al. J Urology 160: 2396-2401, 1998), none inhibit the enzymatic activity of PSMA and few recognize conformational determinants on PSMA.

### Example 2

### Production of Anti-PSMA mAbs

To accurately and quantitatively assess the therapeutic potential of these mAbs, the mAbs are produced in a quantity and quality suitable for extensive in vitro and in vivo characterization. Briefly, the mAb-secreting hybridomas are cultured in roller bottles in DMEM/F12 medium supplemented with 10% FBS that has been depleted of bovine IgG (Life Technologies). During the production phase of the culture, cells are maintained at ˜5×10⁶ cells/mL via twice-weekly exchanges of media. Collected media are clarified by filtration through a 0.22 micron filter and stored at −95° C. prior to purification. Given an average antibody expression levels of ˜25 mg/L, approximately 3L of roller bottle supernatants are required for each antibody to allow for losses in purification.

Culture supernatants from a given hybridoma are pooled and loaded onto a Protein A Sepharose affinity column. Mouse IgG2a, mouse IgG2b and human IgG1 antibodies are loaded directly, but supernatants containing mouse IgG1 antibodies are adjusted to pH 8.5 and 1M NaCl prior to loading in order to promote binding. After washing the column, the mAb is eluted with low pH buffer into fractions using 1M Tris, pH 8.0. Elution peak fractions are pooled, dialyzed against PBS buffer, concentrated to 5 mg/mL and stored in sterile aliquots at −95° C. All purification procedures are carried out using endotoxin-free buffers and sanitized chromatography columns. Purified mAbs are tested for purity by reducing and nonreducing SDS-PAGE, for PSMA binding affinity by ELISA, and for endotoxin levels by the limulus amebocyte lysate assay. These procedures routinely yield “animal-grade” antibody at >95% purity and <0.5 endotoxin units per milligram of protein.

### Example 3

### Evaluation of the Therapeutic Potential of the Unlabeled mAbs In Vitro

Purified mAbs are tested in a battery of assays for therapeutically relevant properties, including affinity, specificity, enzyme inhibitory activity and effector functions. The ideal product candidate binds and inhibits PSMA activity at subnanomolar concentrations and mediates potent cell-killing through Fc-related effector functions.

First, the mAbs' affinity for cell-surface and secreted forms of PSMA is measured by flow cytometry and ELISA, respectively. In the flow cytometry assay, varying amounts of mAbs are incubated with 5×10⁵ 3T3-PSMA cells in FACS buffer (PBS containing 1% FBS and 0.1% NaN₃) for 2 hr to allow for saturation binding. Cells are washed and incubated with a phycoerythrin-coupled goat antibody to mouse IgG (ICN/Cappel) for detection of bound mAb by flow cytometry. Specific binding is calculated by subtracting the fluorescence intensity observed with parental 3T3 cells.

For ELISA, CHO cell-derived recombinant soluble PSMA protein (rsPSMA, Progenics, Tarrytown, NY) is diluted to 1 μg/ml in 50 mM carbonate buffer, pH 9.4, and coated overnight at 4° C. onto 96-well Immulon II microtiter plates at 100 μl/well. The plates are then blocked for 2 hr with PBS buffer containing 5% BSA. mAbs are added in a range of concentrations in ELISA buffer (PBS buffer containing 2% BSA, 1% FBS and 0.5% Tween 20) for 2 hours at room temperature. The plates are washed, and horseradish peroxidase conjugated goat antibody to mouse IgG is added for 1 hr at room temperature. The plates are washed again and 3,3′,5,5′-tetramethylbenzidine dihydrochloride (TMB) substrate (Pierce, Rockford, Ill.) is added for colorimetric readout at 450 nm using an ELISA plate reader (Molecular Devices, Sunnyvale, Calif.).

### Example 4

### mAb Cross-Competition Binding Assay

To identify whether a given group of mAbs recognize distinct or overlapping epitopes on PSMA, cross-competition binding assays are performed (Liu, H. et al. Cancer Res 57: 3629-3634, 1997). In this flow cytometry assay, a biotinylated test mAb is incubated with 3T3-PSMA cells in the presence or absence of varying concentrations of unlabeled competitor mAbs as described above. Following washing, phycoerythrin-conjugated streptavidin is added to determine the amount of bound biotinylated mAb. The percent inhibition is defined relative to that observed in the presence of an isotype-matched mAb of irrelevant specificity (0% inhibition) and to that observed using excess unlabeled test mAb (100% inhibition).

### Example 5

### Effects of mAbs on PSMA Enzymatic Activity

PSMA has been shown to possess both folate hydrolase (pteroyl-glutamyl carboxypeptidase) and N-acetylated α-linked acidic dipeptidase (NAALADase) enzymatic activities, which may influence the proliferation and malignancies of the tumor cell (Heston, W. D. W. Prostate: Basic and Clinical Aspects (R. K. Naz, ed.). CRC Press, New York: 219-243, 1997). A first set of mAbs described above (mAb 3.9, mAb 5.4 and mAb 7.3) and mAb J591 (ATCC #HB-12126) were tested for folate hydrolase modulating activity using previously described assays for measuring PSMA enzymatic activity (Pinto, J. T. et al. Clinical Cancer Res 2: 1445-1451, 1996).

Briefly, folate hydrolase activity was measured as follows. Fifty μM methotrexate di-gamma glutamate and 10 μg/ml rsPSMA (premixed with anti-PSMA or irrelevant mAb) was incubated in pH 4.5 acetate buffer in a volume of 100 μl for 2 hr at 37° C. Reactions were terminated by boiling for 5 minutes prior to separation of free, mono- and di-gamma glutamate forms of methotrexate by capillary electrophoresis on a Spectra Phoresis 1000 (Thermo Separation, San Jose, Calif.). The various methotrexate derivatives were quantified based on their retention times and absorbance at 300 nm.

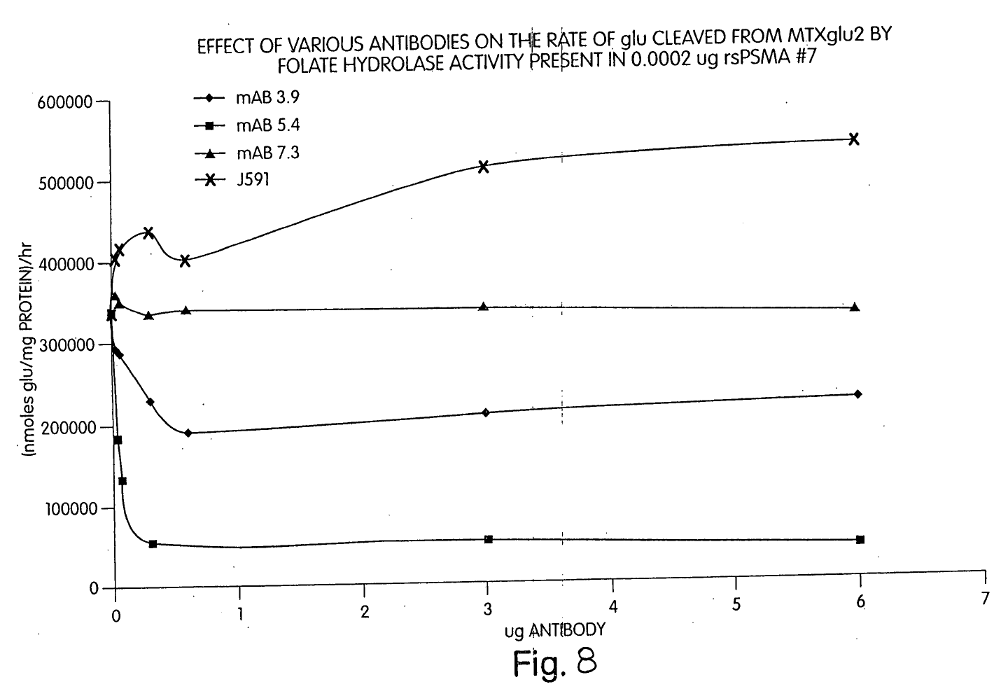

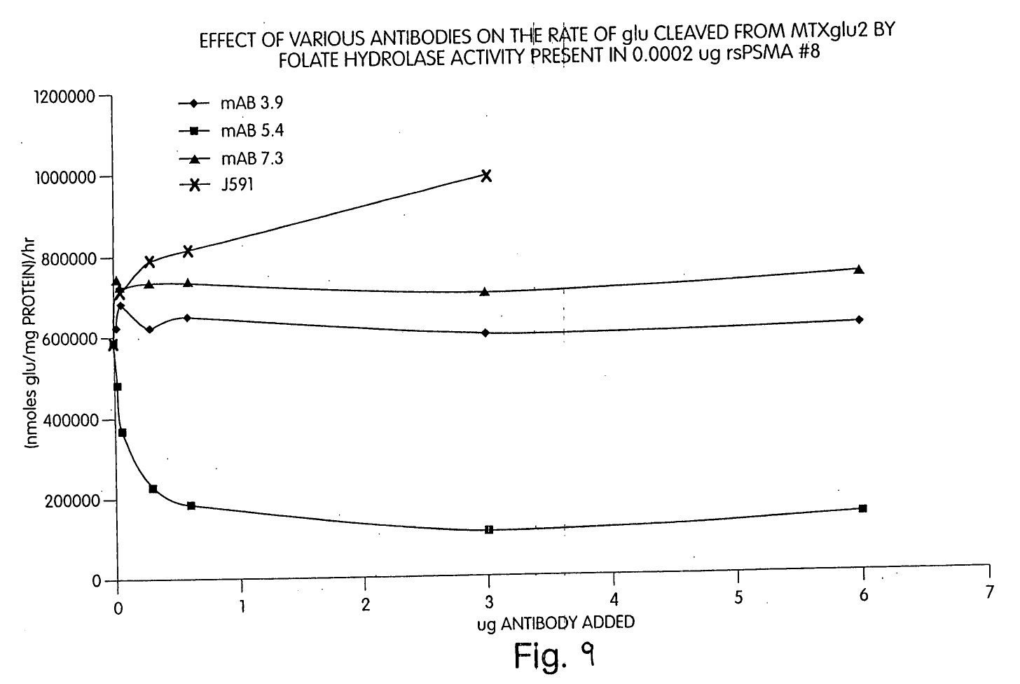

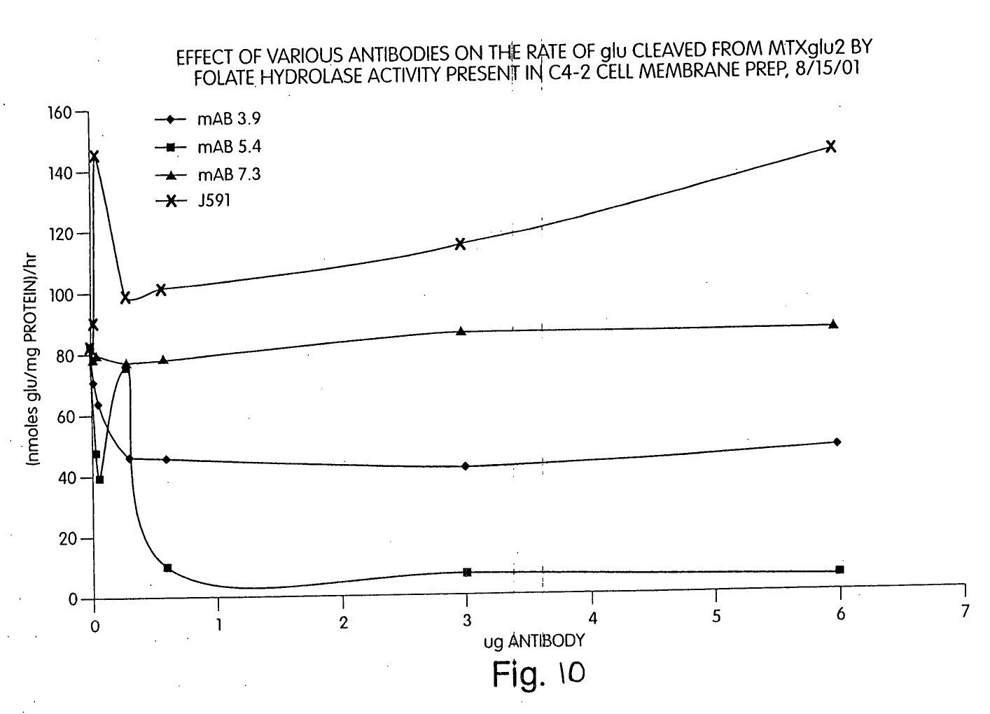

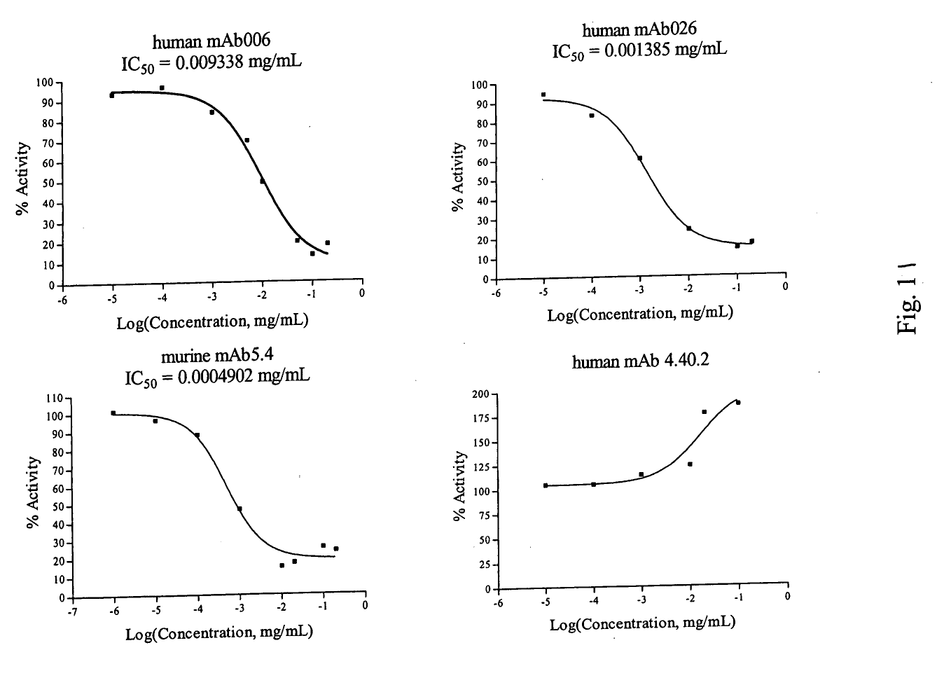

The data show that mAb 5.4 potently blocks the enzymatic activity of purified rsPSMA protein and in lysates of C4-2 cells. C4-2 is an androgen independent derivative of the LNaCP cell line (human prostate cancer line) which expresses endogenous PSMA. More details regarding the C4-2 cell line maybe found in O'Keefe D. S. et al. Prostate 45: 149-157, 2000). FIGS. 8 and 9 provide the results for two production lots of rsPSMA (rsPSMA #7 and rsPSMA #8). The results for the C4-2 cell lysates are shown in FIG. 10. The figures illustrate the effect of four antibodies (mAb 3.9, mAb 5.4, mAb 7.3 and mAb J591) on the enzymatic activity of folate hydrolase by way of the rate of cleavage of glutamate from methotrexate di-gamma glutamate (MTXGlu2) by folate hydrolase present in the two production lots of rsPSMA and in the C4-2 cell lysates. In addition to the inhibitory effects of mAb 5.4, mAb 3.9 was also found to inhibit folate hydrolase activity.

Another set of mAbs (mAb 4.40.2, mAb 006, mAb 026 and mAb 5.4) was also tested for folate hydrolase modulating activity. The data confirm that mAb 5.4 potently blocks folate hydrolase activity of PSMA (FIG. 11). The concentration of mAb 5.4 which inhibited PSMA enzymatic activity by 50% (IC50, also referred to as EC50 or “effective concentration”) was determined to be 4.902×10⁻⁴ mg/mL. The data further show that mAb 006 and mAb 026 also block PSMA folate hydrolase activity, while mAb 4.40.2 did not (FIG. 11). The IC50 values for mAb 006 and mAb 026 were 9.338×10⁻³ mg/mL and 1.385×10⁻³ mg/mL, respectively.

For NAALADase activity assays, rsPSMA protein is incubated with varying amounts of anti-PSMA or control mAbs in 50 mM Tris pH 7.4, 1 mM CoCl₂ for 10 minutes at 37° C. before adding 50 μl of 0.6 μM N-acetylaspartyl-[³H]glutamate. After 15 minutes, the reaction is stopped by adding 1 ml of 100 mM NaPO₄. Cleaved glutamate is separated from the substrate by ion exchange chromatography and detected by scintillation counting. Each measurement is performed in triplicate.

### Example 6

### Reactivity with Normal and Malignant Human Tissues by Immunohistochemistry

Anti-PSMA mAbs are tested by immunohistochemistry for reactivity with both normal and malignant human tissues using an avidin-biotin peroxidase method (Silver, D. A. et al. Clin Cancer Res 3: 81-85,1997). Frozen or paraffin-embedded tissues can be used. Paraffin-embedded tissue sections are deparaffinized and endogenous peroxidase activity is blocked by incubation with 1% H₂O₂ for 15 minutes. Sections are blocked in a 1:10 dilution of horse serum in 2% PBS-BSA (Sigma Chemical, St Louis, Mo.) for 30 minutes before overnight incubation with 2 μg/ml anti-PSMA mAb in 2% PBS-BSA. After washing, sections are incubated with biotinylated secondary antibody, washed, and incubated with avidin:biotin peroxidase complexes (Vector Laboratories, Burlingame, Calif.) diluted 1:25 in PBS for 30 minutes. After washing, sections are visualized by immersion in PBS containing 0.05% diaminobenzidine tetrachloride, 0.01% H₂O₂, and 0.5% Triton X-100. Negative control sections are incubated with isotype-matched mAbs of irrelevant specificity. As a positive control, 7E11 (Cytogen, Princeton, N.J.), a well-characterized anti-PSMA mAb, is used.

### Example 7

### Antibody-Dependent Cellular Cytotoxicity (ADCC)

In the ADCC assay, mAbs are serially diluted and combined with ⁵¹Cr-labeled 3T3-PSMA cells or human prostate PC-3 cells that have been engineered to express human PSMA (PC-3-PSMA cells). NK effector cells are purified from lymph nodes or spleens using anti-NK microbeads (Miltenyi Biotec). Sera, NK effector cells, and ⁵¹Cr-loaded target cells are co-incubated at effector:target cell ratios of 10:1, 20:1, and 40:1, with each condition performed in triplicate. Cells are incubated 4-5 hours at 37° C. before supernatants are collected for measurement of ⁵¹Cr release by gamma counting. The percent specific lysis is determined relative to that observed in the presence of isotype-matched non-specific mAb (0% lysis) to that obtained using 10% sodium dodecyl sulfate (100% lysis).

### Example 8

### Complement-Mediated Lysis (CML)

For CML, ⁵¹Cr-loaded 3T3-PSMA or PC-3-PSMA cells serve as target cells. Serial dilutions of mAbs are co-incubated with rabbit complement and target cells for 4-5 hours at 37° C., with each condition being performed in triplicate. Supernatants are then collected and counted with a gamma counter. Specific lysis is computed as previously done with the ADCC assay data.

### Example 9

### Anti-Proliferative Effects

To test anti-proliferative effects of these antibodies, anti-PSMA mAbs are serially diluted and incubated with LNCaP, PC-3-PSMA and parental PC-3 cells in log-phase growth. At 4 hr, 24 hr, and 72 hr intervals, cells are removed and analyzed for density and viability by trypan blue staining and WST-1 assay (Roche Biochemicals).

### Example 10

### Optimization of Chelation and Radiolabeling Procedures

The most promising mAbs identified using the procedures described in the foregoing examples will be optimized for biochemical and biological stability and activity after labeling prior to evaluation in animals. Success in in vitro experiments is defined as identification of a radiolabeled mAb that specifically kills PSMA-expressing tumor cells at >10-fold lower concentrations than unlabeled or similarly labeled isotype control mAb.

Because the preferred α- and β-emitting isotopes are all radiometals, each of the mAbs is first conjugated with an appropriate metal chelating agent. Based on the favorable in vivo stability data and its proven use in human clinical trials, the bifunctional chelating agent C-functionalized trans cyclohexyldiethylenetriaminepentaacetic acid (p-SCN-CHX-A″-DTPA) is the preferred agent for attaching either ⁹⁰Y or ²¹³Bi to the antibody (Brechbiel, M. W. et al. J. Chem. Soc. Chem. Commun. 1169-1170, 1991). A form of this chelate has previously been tested in more than 70 doses in humans in ongoing trials at Memorial-Sloan Kettering Cancer Center (McDevitt, M. R. et al. J. Nucl. Med. 40:1722-1727, 1999). For ²²⁵Ac, our initial studies will examine a novel bifunctional chelating agent termed p-SCN-Bz-HEHA (1,4,7,10,13,16-hexaazacyclooctadecane-N,N′,N″,N′″N″″,N′″″hexaacetic acid) (Deal, K. A. et al. J. Med. Chem. 42:2988-2992, 1999). The objective is to optimize the antibody conjugation and chelation ratios to maximize labeling yield and activity while maintaining suitable stability for in vivo utilization. Additional chelating agents also are used as they become available from the N.I.H. and other sources.

Initially, the antibody is rendered metal-free by incubation with a large molar excess of EDTA at pH=5. The EDTA and any metals scavenged from the antibody preparation are removed via continuous buffer exchange/dialysis so as to replace the pH=5 buffer with the conjugation buffer (Nikula, T. K. et al. Nucl. Med. Biol. 22:387-390, 1995). Conditions that yield optimal chelator to antibody ratio but still remain immunoreactive are identified by systematically varying the chelator: antibody ratio, reaction time, temperature, and/or buffer systems about initial conditions that employ a 40-fold molar excess of chelator to antibody in HEPES buffer, pH 8.5. The number of chelates bound per antibody is determined using an established spectrophotometric method (Pippin, C. G. et al. Bioconjugate Chemistry 3: 342-345, 1992).

For ⁹⁰Y and ²²⁵Ac constructs, labeling efficiency is measured directly. For ²¹³Bi, initial antibody constructs are tested for chelation efficiency using ¹¹¹In, which has similar chelation chemistry as ²¹³Bi but possesses the advantages of a longer half life (t_{1/2}=3 days), ready availability, and traceable γ-emission. Once optimized using ¹¹¹In, labeling efficiency is determined for ²¹³Bi.

Radiolabeled mAb is purified over a BioRad 10DG desalting column using 1% HSA as the mobile phase and evaluated by instant thin layer liquid chromatography (ITLC) and/or high performance liquid chromatography (HPLC) to determine the percent incorporation of radionuclide (Zamora, P. O. et al. Biotechniques 16: 306-311, 1994). ITLC and HPLC provide a means of establishing purity and identifying the percent of low molecular weight radiochemical impurities (i.e., metal chelates, colloids, and free metal). Duplicate ITLC strips for each mobile phase are developed, dried, and cut at the R_{f} of 0.5 mark and counted in a gamma counter. The HPLC system is equipped with both an online UV absorption detector and radioactivity detector. The HPLC elution profile directly correlates radioactivity with protein and low molecular weight species as a function of the elution time. A TSK SW3000_{XL} column (TosoHaas, Montgomeryville, Pa.) is used and calibrated using a range of protein molecular weight standards.

### Example 11

### Affinity and Immunoreactivity of Radiolabeled mAbs

Once radiolabeled constructs are obtained, purified, and assessed for biochemical and radiochemical purity, biological activity is determined. Binding activity of the radioconstruct is performed by Scatchard analysis of binding data obtained using whole LNCaP and 3T3-PSMA cells and/or membrane fractions as previously described (Scheinberg, D. A. et al. Leukemia 3: 440-445 (1991).

The immunoreactivity of the synthetic constructs is evaluated in order to correlate the chelate:antibody molar ratio with the biological activity. Briefly, 2 ng of labeled mAb is incubated with a 25-fold excess of PSMA as expressed on 3T3-PSMA cells. After a 30 min incubation at 0° C., the cells are collected by centrifugation and the supernatant containing unbound mAb is added to fresh 3T3-PSMA cells for an additional 30 min at 0° C. Both sets of cells are centrifuged and washed twice with cold PBS. The cell pellets, supernatant and wash fractions are counted for radioactivity. Immunoreactivity is defined as the amount of radioactivity in the cell pellets divided by the total radioactivity in the cell pellets, supernatant and wash fractions.

### Example 12

### mAb Internalization

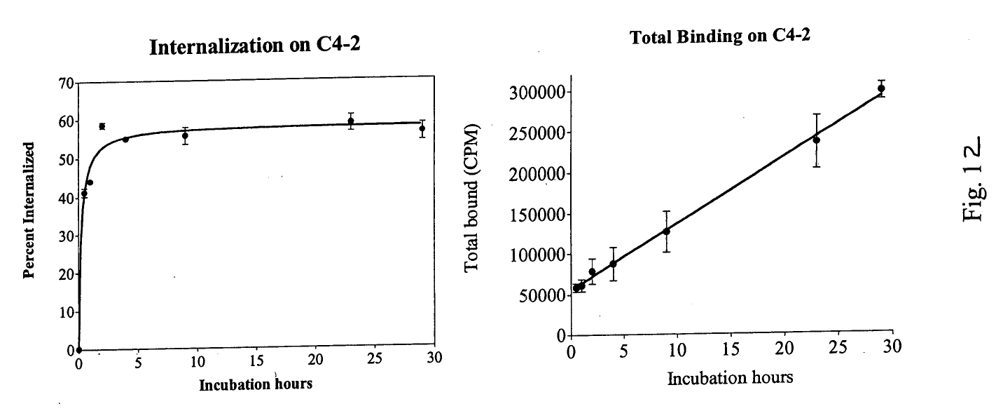

The activity of radiolabeled mAbs can be significantly modulated by their internalization rates. Based upon previous results by other groups (Smith-Jones P. M. et al. Cancer Res 60: 5237-5243, 2000), significant internalization of PSMA after binding with one or more of the mAb constructs was expected. Internalization of the cell surface antibody-antigen complex was measured using ¹¹¹In radiolabeled antibody (mAb 026) constructs (Caron, P. C. et al. Cancer Res 52: 6761-6767, 1992). Briefly, 5×10⁵ C4-2 cells were incubated at 37° C. in 5% CO₂ with ¹¹¹In radiolabeled antibody. At different times, cells were washed with PBS and cell-surface bound radiolabeled constructs were stripped with 1 ml of 50 mM glycine/150 mM NaCl, pH=2.8. Total cell-associated radioactivity and acid-resistant (internalized) radioactivity were determined by γ-counting. Percent internalization and total binding were calculated. ¹¹¹In labeled mAb 026 was found to be rapidly and efficiently internalized. FIG. 12 shows the percent internalization and total binding of ¹¹¹In labeled mAb 026 as a function of incubation time. Cells (such as parental 3T3 cells) that do not express PSMA can be used as a control to determine non-specific binding.

### Example 13

### In Vitro Cytotoxicity Studies

Assessment of in vitro cytotoxicity of α-labeled mAbs was undertaken once the immunoreactivity of the radioimmunoconjugate was established. Approximately 50,000 target cells (either LNCaP or 3T3-PSMA cells) were treated in 96 well plates and analyzed 24-96 hours later. Quantification of cell death due to ²²⁵Ac-labeled constructs (or ²¹³Bi) was accomplished by determining the uptake of ³H-thymidine by surviving cells (Nikula, T. K. et al. J. Nucl. Med. 40: 166-176, 1999). Specificity was determined by use of control cells (PSMA-negative human prostate cell lines PC-3 and DU-145, as well as control 3T3 cells), blocking with excess unlabeled antibody, and control radioconjugates.

The cytotoxic effects of antibody conjugate concentration, specific activity, and time of exposure were then assessed. Cytotoxicity was expressed relative to that seen with 1M HCl (100% cell death) and media (background cell death). LD₅₀ values were calculated by plotting cell viability as a function of the number of ²²⁵Ac atoms bound on the cells (McDevitt, M. R. et al. (1998) Eur. J. Nucl. Med. 25: 1341-1351 (1998).

Multicellular spheroids of LNCaP-FGC cells had been established and were used to investigate the potential of radioimmunotherapy (RIT) to eradicate minimal disease in vitro. These three-dimensional spheroids mimic tissue structures more accurately than monolayer cultures and thus provide a more relevant model of solid tumors (O'Connor, K. C. Pharm. Res. 16: 486-493, 1999). LNCaP-FGC is a fast growing clone of the original LNCaP cell line, and the cells were grown using a liquid overlay technique to a size of 200-600 μm (Ballangrud, A. M. et al. Clin. Cancer Res. 5: 3171s-3176s, 1999). In larger spheroids, the inner mass of cells becomes necrotic, while the outer rim consists of proliferating tumor cells. Antibody penetration was measured by confocal microscopy, and prior results suggested that an anti-PSMA antibody should penetrate to a depth of 40-50 μm (Ballangrud, A. M. et al. 7th Conference on Radioimmunodetection and Radioimmunotherapy of Cancer, Princeton N.J., 1998). The in vitro cytotoxicity of ²²⁵Ac-3.9 on LNCaP target cells is shown in FIG. 26. The percentage of viable PSMA⁺ LNCaP cells was plotted as a function of activity of the radioconjugate. Addition of a 100-fold excess of unlabeled antibody was used as a control for specificity.

### Example 14

### Evaluation of the In Vivo Efficacy of Unlabeled and Radiolabeled mAbs in Mouse Xenograft Models of Human Prostate Cancer

Antibodies that are successful in the foregoing assays demonstrate significant specificity and functional properties that suggest they will be useful for therapeutic use. The most promising of these radiolabeled and “naked” mAb constructs are evaluated in the best available mouse models of prostate cancer. The studies employ an established xenograft model in which the LNCaP human prostate tumor cell line is injected into immunocompromised nude mice and allowed to form solid tumors (Ellis, W. J. et al. Clin Cancer Res 2: 1039-1048 (1996), which then are treated with both radiolabeled and unlabeled anti-PSMA mAb constructs. Follow-on studies also utilize a mouse xenograft model, CWR22, which reproduces many of the key biological features of human prostate cancer.

Lncap Tumor Cell Xenograft Model

A construct showing high affinity and high specificity is taken into the LNCaP tumor cell xenograft in vivo model for biodistribution and pharmacokinetic analysis. ¹¹¹In-labeled anti-PSMA antibody is used for these studies due to its favorable chelation chemistry, radioactive half-life and traceable gamma emission. Timepoints are evaluated as appropriate for the half-lives of ²¹³Bi, ²²⁵Ac, ¹⁷⁷Lu and ⁹⁰Y, which are the nuclides of therapeutic interest. Labeled radioconstructs (1-5 μg) are injected i.v. into nude mice (normal and tumor bearing) and the mice are sacrificed at 5 min, 15 min, 30 min, 60 min, 2 hrs, 4 hrs, 18 hrs, and 24 hrs post-injection. Blood and major organs are taken from animals, weighed, and the percent radioactivity injected per gram of tissue is determined (Nikula, T. K. et al. J. Nucl. Med. 40: 166-176, 1999). Specificity is addressed by pre-injection with excess unlabeled construct. Macroscopic tumor volume and animal survival rates is recorded throughout the experiments.

A dose-ranging study is also conducted to determine the toxicity of the constructs when administered via i.v. or i.p. injection to normal and tumor-bearing mice. These animals are routinely examined for toxic side effects during the course of the studies by blood chemistry and physical examination. Animals are sacrificed during and at the conclusion of the study in order to collect blood and body tissues for further evaluation. Previous data has demonstrated an approximate maximum tolerated dose of 250 μCi/mouse, so total doses are kept below that level.

Once i.v. biodistribution and toxicity is documented, radiotherapy of tumors is assessed. Groups of five mice are injected with <1 μg radiolabeled anti-PSMA mAb construct both pre- and post-tumor challenge to assess anti-tumor activity. Antigen negative (RAJI or RAMOS) xenografted tumors are also used as a control. Other controls include (1) treatment with unlabeled anti-PSMA mAb only and (2) excess unlabeled anti-PSMA mAb pretreatment before ²¹³Bi, ²²⁵Ac, ¹⁷⁷Lu and/or ⁹⁰Y-labeled anti-PSMA to block specific targeting.

Groups of tumor bearing mice are injected with unlabeled anti-PSMA mAbs (at equimolar concentrations) and several dose levels of radiolabeled anti-PSMA or a similarly labeled isotype control antibody. The effect on tumor growth is assessed over time. Statistical differences between therapy groups is determined using an analysis of variance (ANOVA) method and animal survival is illustrated using Kaplan-Meier plots. The efficacy of ²¹³Bi, ²²⁵Ac, ¹⁷⁷Lu and/or ⁹⁰Y-labeled anti-PSMA constructs is correlated to the data obtained in vitro. Success in these experiments is defined as the ability to significantly (p<0.05) increase life-span and/or decrease tumor volume as compared to a radiolabeled isotype control mAb.

Furthermore, the tumor models are used to test whether predosing with unlabeled antibody prior to injection of radiolabeled antibody improves delivery of the radiolabeled antibody to the tumor. The tumor-bearing mice are injected with <1 μg radiolabeled anti-PSMA antibody with or without a prior single injection of 5-100 μg of unlabeled antibody. After several days, animals are sacrificed for evaluation of the distribution of radioactivity in the tumor, normal tissue, and blood. If predosing with unlabeled antibody improves delivery and targeting of radiolabeled antibody to the tumors, this approach is applied and optimized in toxicity and therapeutic studies.

In addition to overall survival, the role of timing of the injection after tumor transplantation (Day 1 vs 3 vs 7), the role of dosage (dose-response curves using 3-4 dose levels), the role of schedule (single vs multiple divided daily injections) and the specificity of the treatment (pre-treatment with unlabeled anti-PSMA to block targeting) is examined.

These in vivo studies are designed to address the maximum tolerated dose of radiolabeled antibody, the activity of the antibody, the optimal dosing schedule (single or multiple injections), and the effect on tumor size. Successful completion of this work enables determination of the feasibility of PSMA-targeted alpha particle radioimmunotherapy (RIT) of prostate cancer and identifies the optimal ²¹³Bi and/or ²²⁵Ac-labeled constructs to enter into clinical development.

CWR22 Mouse Xenograft Model

The most promising anti-PSMA mAbs in unlabeled, toxin-labeled and/or radiolabeled form are tested in the CWR22 human prostate cancer xenograft mouse model, (Wainstein, M. A. et al. Cancer Res 54:6049-6052 (1994); Nagabhushan, M. et al. Cancer Res 56:3042-3046 (1996); Pretlow, T. G. et al. J Natl Cancer Inst 85:394-398 (1993)). This model has many features of the human condition including a dependence on androgens, a correlation between measured levels of PSA in serum and tumor size, and high-level expression of PSMA. Following androgen withdrawal, PSA levels decrease to nearly undetectable levels and tumor volume decreases. Later, the tumor regrows as an androgen-independent neoplasm, manifest initially by a rise in PSA and later, measurable tumor growth. After androgen withdrawal, tumors regrow at variable time periods.

Four to six week old nude athymic BALB/c male mice are obtained from the National Cancer Institute-Frederick Cancer Center and maintained in pressurized ventilated caging. While immunodeficient in many respects, these mice mediate wild-type levels of ADCC and CML. The CWR22 tumor line is propagated in the animals by the injection of minced tumor tissue from an established tumor into the subcutaneous tissue of the flanks of athymic nude mice together with reconstituted basement membrane (Matrigel, Collaborative Research, Bedford, Mass.). To maintain serum androgen levels, the mice are administered 12.5-mg sustained-release testosterone pellets (Innovative Research of America, Sarasota, FL) subcutaneously before receiving tumors. Three to four weeks after inoculation, tumors of approximately 1.5×1.0×1.0 cm are measured. Androgens are withdrawn by surgical castration under pentobarbital anesthesia and removal of the sustained-release testosterone pellets. Tumor size is determined by caliper measurements of height, width and depth. PSA values are performed on the serum of the mice after tail bleeding using a Tandem-R PSA immuno-radiometric assay (Hybritech, San Diego, Calif.).

Groups of five mice are injected with anti-PSMA mAb or a similar isotype control mAb at dosages from 5-100 μg to assess anti-tumor activity. The effect of scheduling single doses vs. multiple divided daily injections is also examined. Macroscopic tumor volume and animal survival rates are recorded throughout the experiments. Statistical differences between therapy groups are determined using an analysis of variance (ANOVA) method and animal survival are illustrated using Kaplan-Meier plots, with success defined as a difference of p<0.05. Similarly, the efficacy of “naked” mAbs is compared to that seen with ⁹⁰Y, ¹⁷⁷Lu, ²¹³Bi and/or ²²⁵Ac-labeled anti-PSMA constructs.

These in vivo studies are designed to address the maximum tolerated dose of mAb, the activity of the antibody, the optimal dosage and dosing schedule (single or multiple divided injections), and the effect of treatment on tumor size. Successful completion of this work will enable determination of the feasibility of PSMA-targeted immunotherapy of prostate cancer and identification of the optimal constructs to enter into clinical development.

### Example 15

### Investigation of Native PSMA Protein Conformation

Extraction of PSMA from the Cell Surface of LNCaP and 3T3 Cells

LNCaP or 3T3 cells were grown to confluency in a T150 cell culture flask, detached using cell dissociation solution (Mediatech, Herndon, Va.) and transferred to a 15 ml conical tube. The cells were washed twice with PBS and resuspended with 2 ml of M-Per™ Mammalian Protein Extraction Reagent (Pierce, Rockford, Ill.). Following incubation for 10 min at 4° C., cell debris and insoluble aggregates were removed by centrifugation at 15,000 rpm for 30 min at 4° C. The supernatant was transferred to a cryogenic vial and stored at −80° C. until further use.

Production of Recombinant, Soluble PSMA (rsPSMA)

The extracellular domain of PSMA (amino acids 44-750 of the full-length protein, SEQ ID NO:1) was obtained as a secreted protein from a DXB11 Chinese hamster ovary (CHO) cell line, stably transfected with an rsPSMA expression vector. The cells were grown in a Celligen Plus 2.2L Packed Bed Bioreactor (New Brunswick Scientific, Edison, NJ) in protein-free media. The Bioreactor was operated in perfusion mode, and supernatant was collected aseptically into collection bags maintained at 4° C. The protease inhibitor aprotinin was added to the harvest supernatant, which was concentrated 25-fold prior to storage at −90° C. In some instances for purification, the concentrate was thawed and purified using subsequent steps of Concanavalin A lectin affinity chromatography and Butyl-Sepharose hydrophobic interaction chromatography or according to the steps shown below.

The purified rsPSMA protein is dimeric, and possesses folate hydrolase enzymatic activity when tested according to published procedures (Pinto et al., Clinical Cancer Research 2:1445, 1996) and reacts with each of a panel of conformation-specific monoclonal antibodies, indicating that rsPSMA adopts a native conformation.

Purification of Recombinant, Soluble PSMA (rsPSMA)

Cell culture supernatants were concentrated 25-fold by tangential flow ultrafiltration and adjusted to 35% saturation with ammonium sulfate. Under these conditions, rsPSMA remains in the supernatant. Precipitated proteins were removed by centrifugation (20,000×g for 30 min, SS-34, Sorvall) and the clarified supernatant was applied to a Butyl-Sepharose resin (BioRad, Hercules, Calif.) followed by a wash with 35% ammonium sulfate in neutral phosphate-buffered saline containing 1 mM Ca²⁺ and 0.5 mM Mg²⁺ (PBS+). rsPSMA eluted in the flow-through and wash fractions of the column. The fractions containing the rsPSMA protein were pooled, dialyzed into 10 mM sodium phosphate, pH 7.0, and loaded onto a Ceramic Hydroxyapatite column (BioRad, Hercules, Calif.). rsPSMA was eluted from the resin using 2M sodium chloride in 10 mM sodium phosphate, pH 7.0. The fractions containing the protein were pooled, dialyzed into 20 mM Tris, pH 7.5 containing 1 mM Ca²⁺ and 0.5 mM Mg²⁺, and applied to a Q650-Sepharose column (TosoHaas, Montgomeryville, Pa.). rsPSMA was eluted from the resin with 150 mM NaCl in 20 mM Tris, pH 7.5 containing 1 mM Ca²⁺ and 0.5 mM Mg²⁺. Monomeric and dimeric forms of rsPSMA present after this step were separated using preparative size exclusion chromatography on a Superdex 200 resin (Amersham Biosciences, Piscataway, N.J.) and PBS+ (containing 1 mM Ca²⁺ and 0.5 mM Mg²⁺) as the running buffer. Purified rsPSMA was stored at −80° C. in PBS+. Unless otherwise indicated, PSMA monomers represent spontaneously dissociated protein recovered over SEC rather than forcibly denatured material.

Polyacrylamide Gel Electrophoresis (PAGE) and Western Blotting of the Different PSMA Proteins

For each individual PAGE analysis, 15 μl of each cell lysate and 5 μl of the purified rsPSMA were used.

SDS-PAGE was performed using standard procedures. Samples were prepared by boiling for 5 minutes in the presence of Laemmli sample buffer (with or without the reducing agent dithiothreitol [DTT]). Samples were then applied on a 4-15% Tris-Glycine gel (BioRad, Hercules, Calif.). After electrophoresis for 1 h at 200V, the proteins were transferred onto nitrocellulose (BioRad) and analyzed by Western blotting.

The oligomeric nature of the different PSMA proteins was analyzed using Blue Native PAGE (BN-PAGE). Each sample was diluted with an equal volume of 2×BN-PAGE sample buffer (0.1M MOPS/0.1M Tris/40% glycerol/0.1% Coomassie G-250) prior to loading onto the gel. BN-PAGE was performed using 4-12% BisTris gels (Invitrogen, Carlsbad, Calif.) and 50 mM MOPS/50 mM Tris, pH 7.7 as running buffer. Coomassie Blue was omitted from the cathode buffer to avoid interference with protein binding during the transfer of the proteins onto nitrocellulose. Following electrophoresis for 2.5 hrs at 125V, the proteins were transferred onto a nitrocellulose membrane (BioRad) and analyzed by Western blotting.

Western blotting was performed as follows: Subsequent to transfer, the nitrocellulose membrane was blocked with 5% milk in PBS/0.1% Triton X-100/0.02% SDS, which was also used for the subsequent wash and antibody incubation steps. PSMA proteins were detected using the anti-PSMA mAbs 3.1 or 3.9 (Progenics Pharmaceuticals) as primary antibody and HRP-labeled anti-mouse IgG as secondary antibody and 1 h incubation at room temperature. The membranes were colorimetrically developed using chemiluminescence (NEN Plus, Perkin Elmer Life Sciences, Boston, Mass.).

Analytical size exclusion chromatography (SEC) was performed using a TSK G3000SW_{XL} (TosoHaas, Montgomeryville, Pa.) column equilibrated in PBS+. The column was calibrated using bovine serum albumin (67 kDa), immunoglobulin G (150 kDa), ferritin (440 kDa) and thyroglobulin (670 kDa) as standards.

Results

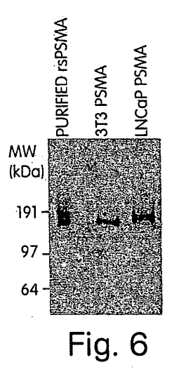

Both full-length PSMA and recombinant, soluble PSMA (rsPSMA) migrated on reducing and non-reducing SDS-PAGE with a molecular weight of 100 kDa (FIG. 5). Thus, like full-length PSMA, rsPSMA is a monomer in the presence of denaturing agents, and no disulfide or other covalent bonds are present to mediate oligomerization. The result for full-length PSMA is in accordance with prior observations (Israeli et al., U.S. Pat. No. 5,538,866; Murphy et al., U.S. Pat. No. 6,158,508; Israeli, et al., Cancer Research 54:1807, 1994; Troyer et al. Int. J. Cancer 62:552, 1995; Troyer et al., The Prostate 30:233, 1997; Grauer et al., Cancer Research 58:4787, 1998). In each of these reports, full-length PSMA migrated as a major band of 100-120 kDa, with a minor (typically <5% of the total PSMA protein) 180-200 kDa band observed in a subset of reports (U.S. Pat. No. 6,158,508; Troyer et al., 1995; Troyer et al., 1997). Troyer et al. (1995) describe the 180-200 kDa species as being a noncovalently associated PSMA dimer that can be disrupted with increasing concentrations of SDS detergent.

rsPSMA contains 94% (707 of 750) of the amino acids present in full-length PSMA, and the two proteins were not clearly resolved in this analysis, as expected.

SDS-PAGE allows the analysis of denatured proteins only. In order to examine native proteins in their native state, other techniques have to be employed, such as Blue Native PAGE (BN-PAGE). BN-PAGE is used to determine the native molecular weight of proteins and their noncovalent complexes (Schägger & v. Jagow, Anal. Biochem. 199:223-231, 1991; Schägger et al., Anal. Biochem. 217:220-230, 1994). The dye Coomassie Blue G-250 binds to the hydrophobic domains on the surface of most proteins, enhances solubility, and introduces a charge shift on the native proteins resulting in migration towards the anode at pH 7.5 irrespective of the isoelectric point of the protein. Although the migration velocity of proteins in BN-PAGE varies somewhat, the molecular mass of proteins can be determined by their respective end points of migration due to the decreasing pore size of the acrylamide gradient present in the gels.

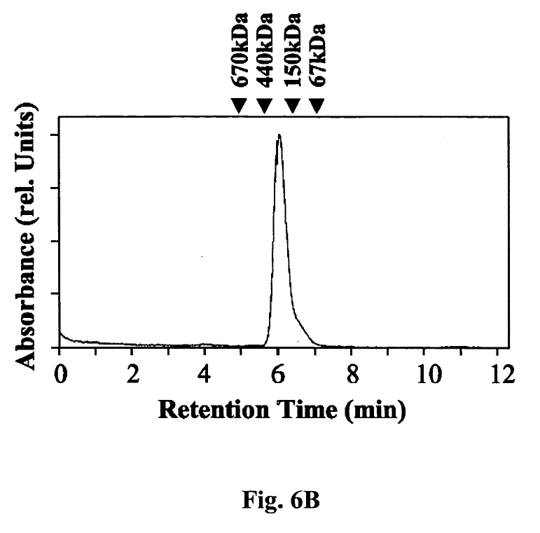

When analyzed by BN-PAGE, full-length PSMA (extracted from LNCaP or 3T3 cells with nonionic detergents) as well as purified rsPSMA migrate with a molecular weight of ˜190 kDa (FIG. 6A). This surprising observation for full-length PSMA indicates that the predominant form of cell-surface PSMA is a noncovalently associated dimer. This unexpected result can be contrasted with that of previous reports (U.S. Pat. No. 6,158,508; Troyer et al. 1995; Troyer et al., 1997), where the PSMA dimer represents a minor species in SDS-PAGE analyses. Presumably, the noncovalent PSMA dimer is largely dissociated by boiling in the presence of the denaturing detergent SDS.

Moreover, the result for the purified rsPSMA protein indicates that the dimer is stabilized via interactions between extracellular amino acids in addition to or exclusive of amino acids in the transmembrane or intracellular segments, which are not present in rsPSMA.

rsPSMA was subjected to analytical size exclusion chromatography (SEC) as a second sizing method. When analyzed in neutral PBS+buffer, purified rsPSMA eluted as a single major peak with an apparent molecular mass of 260 kDa (FIG. 6B), slightly higher than expected. However, glycoproteins (such as rsPSMA) are typically nonglobular in shape and run at higher apparent molecular mass than standard SEC calibration proteins (Schulke, N., et al. (2002) J. Virol. 76, 7760-7776). Therefore, an apparent molecular mass of 260 kDa is consistent with the proposed homodimeric structure of rsPSMA. In contrast, purified monomeric rsPSMA eluted with an apparent molecular mass of 130 kDa. The studies demonstrate that the extracellular domain of PSMA is sufficient for dimerization, and the similarities between rsPSMA (amino acids 44-750) and PSM′ (amino acids 58-750) suggest that the latter protein is likely to dimerize as well.

### Example 16

### Homodimerization is Required for Enzymatic Activity

Enzyme Assays

Pteroyl γ-glutamyl carboxypeptidase (folate hydrolase) activity was determined by monitoring the cleavage of poly γ-glutamylated methotrexate as described (Pinto, J. T., et al. (1996) Clin. Cancer Res. 2, 1445-1451) with the following exceptions. Di-γ-glutamylated methotrexate (MTXglu2) was used as substrate and HPLC was used rather than capillary electrophoresis. At the completion of the incubation (50 μM methotrexate di-gamma glutamate and 10 μg/ml rsPSMA in pH 4.5 acetate buffer in a volume of 100 μl for 2 hr at 37° C.), 100 μl of 0.5 M Na₂HPO₄ was added to stop the reaction. Samples were loaded at a flow rate of 1.25 mmin through a 50×4.6 mm, 3 μm PRISM reversed-phase column (Thermo Hypersil-Keystone, Bellefonte, Pa.) with a PRISM 10×4-mm guard column, eluted with 15% methanol in 85% 0.5M K₂HPO₄, pH 7.0, and quantitated based on relative peak area observed at a wavelength of 313 nm.

For NAALADase assays, rsPSMA was incubated with N-acetyl-α-L-aspartyl-L-glutamate for 22 h at 37° C. in the presence of 20 mM sodium phosphate, 50 mM NaCl, 10 mM ZnCl₂, pH 7.1. Released L-glutamic acid was quantitated by using a commercial kit (R-Biopharm, Marshall, Mich.); 2-(phosphonomethyl) pentanedioic acid and Gly-Pro-7-amido-4-methylcoumarin were purchased from Sigma. Porcine kidney dipeptidyl peptidase IV (DPP IV) was used according to the manufacturer's instructions (Sigma).

Results

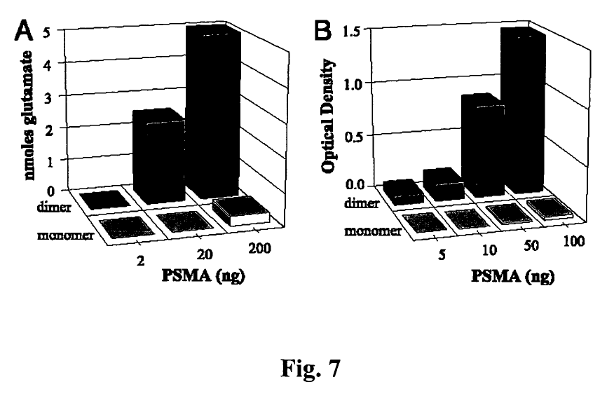

PSMA has been reported to possess folate hydrolase, NAALADase, and DPP IV activities (Pinto, J. T., et al. (1996) Clin. Cancer Res. 2, 1445-1451; Carter, R. E., et al. (1996) Proc. Natl. Acad. Sci. USA 93, 749-753; Pangalos, M. N., et al. (1999) J. Biol. Chem. 274, 8470-8483). The first two activities involve the hydrolysis of a carboxyl-terminal peptide bond to liberate a glutamic acid residue, whereas DPP IV cleaves downstream of an amino-terminal Aaa-Pro dipeptide sequence. The folate hydrolase activities of purified monomeric and dimeric forms of rsPSMA were evaluated. Whereas the dimer demonstrated high-level folate hydrolase activity, the monomer was essentially inactive (FIG. 7A). In fact, the residual activity of the monomer could be attributed to the residual amount (approximately 4%) of dimeric rsPSMA present in the preparation. High-level folate hydrolase activity was also observed for LNCaP cell lysates, consistent with prior observations (Pinto, J. T., et al. (1996) Clin. Cancer Res. 2, 1445-1451). Similarly, dimeric but not monomeric forms of rsPSMA possessed high-level NAALADase activity (FIG. 7B), which was abrogated by using 5 nM of the inhibitor 2-(phosphonomethyl)pentanedioic acid. Neither monomer nor dimer demonstrated DPP IV activity under conditions where porcine DPP IV efficiently hydrolyzed the substrate Gly-Pro-7-amido-4-methylcoumarin. This is consistent with the results reported by Barinka et al. (Barinka, C., et al. (2002) J. Neurochem. 80, 477-487), who similarly failed to confirm the DPP IV activity previously reported for PSMA (Pangalos, M. N., et al. (1999) J. Biol. Chem. 274, 8470-8483).

### Example 16

### Dissociation of PSMA Multimers

PSMA is a putative zinc metalloprotease, and site-directed mutagenesis of amino acids implicated in zinc binding results in a profound loss of enzymatic activity (Speno et al., Molecular Pharmacology, 55:179, 1999). These amino acids include His-377, Asp-387, Glu-425, Asp-453 and His-553. Ethylenediaminetetraacetic acid (EDTA) is a strong chelating agent for Zn²⁺ and other divalent cations, and thus has the potential to remove Zn²⁺ or other coordinate divalent cations from PSMA. We have determined that EDTA treatment causes the PSMA homodimer to dissociate into monomeric subunits. Similar results can be expected for other agents that possess similar chelating properties, such as ethyleneglycol-bis(beta-aminoethyl ether) (EGTA).

The purified rsPSMA protein was incubated with or without 10 mM EDTA for 16 hr at 4° C. and then analyzed by BN-PAGE. Under these conditions, the EDTA-treated protein was monomeric, whereas rsPSMA remained dimeric in the absence of EDTA. Although the dissociation of the PSMA dimer into monomer was essentially complete, any residual dimeric protein can be removed if desired by gel filtration, ultracentrifugation or other size-based separation methods that are well-known to those skilled in the art.

### Example 17

### Methods for Identifying Promoters of PSMA Dissociation

Compounds are screened for the ability to promote dissociation of PSMA dimers using a method that includes:

(a) contacting a PSMA dimer with a compound under conditions that do not promote dissociation of the PSMA dimer in the absence of the compound;

(b) measuring the amount of PSMA monomer; and

(c) comparing the amount of PSMA monomer measured in the presence of the compound with that observed in the absence of the compound.

An increase in the amount of PSMA monomer measured in the presence of the compound indicates that the compound is capable of promoting dissociation of the PSMA dimer.

In a further embodiment, compounds are screened for the ability to promote dissociation of PSMA dimers using a method that includes:

(a) contacting a PSMA dimer with a compound under conditions that do not promote dissociation of the PSMA dimer in the absence of the compound;

(b) measuring the amount of PSMA dimer; and

(c) comparing the amount of PSMA dimer measured in the presence of the compound with that observed in the absence of the compound.

A decrease in the amount of PSMA dimer measured in the presence of the compound indicates that the compound is capable of promoting dissociation of the PSMA dimer.

In a further embodiment, compounds are screened for the ability to promote dissociation of PSMA dimers using a method that includes:

(a) contacting a PSMA dimer with a compound under conditions that do not promote dissociation of the PSMA dimer in the absence of the compound;

(b) measuring the amounts of PSMA monomer and PSMA dimer;

(c) calculating a ratio of PSMA monomer to PSMA dimer; and

(d) comparing the ratio obtained in (c) with that obtained in the absence of the compound.

An increase in the ratio measured in the presence of the compound indicates that the compound is capable of promoting dissociation of the PSMA dimer.

### Example 18

### Cell Surface PSMA Binding Studies

Flow Cytometry

Parent 3T3 cells or PSMA-expressing 3T3 cells (2×10⁵ cells per condition) were washed in PBS and incubated with PBS containing goat serum (10% v/v) for 20 minutes on ice to block non-specific binding sites. Anti-PSMA monoclonal antibodies (unpurified form in supernatants or purified mAbs) were added in serial dilutions to cells in 100 μl PBS and incubated on ice for 30 minutes. Control anti-human IgG (Caltag, Burlingame, Calif.) was used to establish background binding. After two washes in PBS, the cells were incubated with anti-human IgG (BD Pharmingen, San Diego, Calif.) for 30 minutes on ice. Cells were washed twice in PBS, resuspended in 250%1 PBS and analyzed by flow cytometry using a FACScan machine (Becton Dickinson, Franklin Lakes, N.J.) and CellQuest software. Viable cells were gated by forward scatter and side scatter parameters, and binding was quantified using histogram plots of mean fluorescence intensity (MFI) levels.

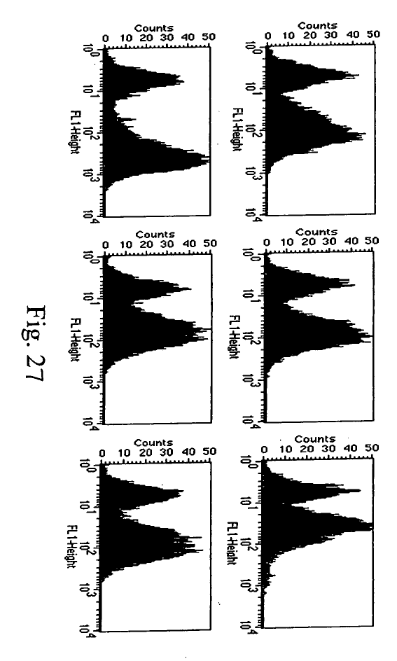

Anti-PSMA mAbs XG-006 (PTA-4403 and PTA-4404, heavy and light chain plasmids), XG-051 (PTA-4407 and PTA-4408), 4.40.1 (PTA-4360; 4.40, 4.40.1 and 4.40.2 are the same antibody that represent different stages of subcloning the hybridoma), 4.49.1, 4.292.1 (PTA-4390) and 4.304.1 were found to avidly bind to cell surface PSMA (FIG. 27).

Maximal Binding

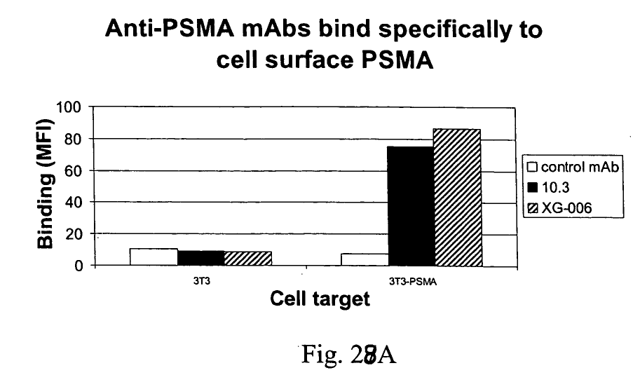

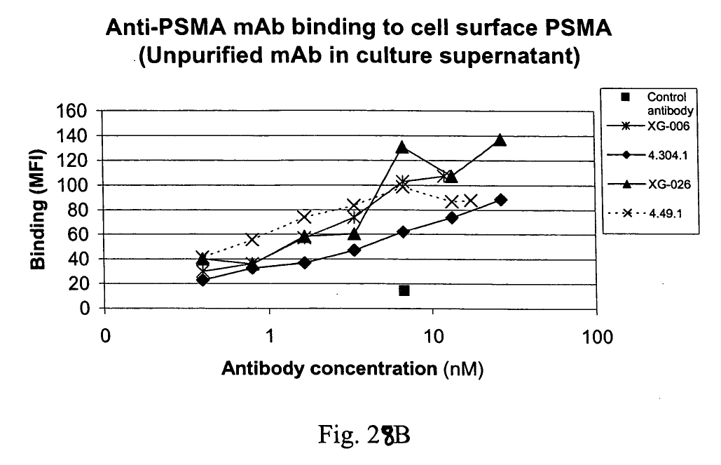

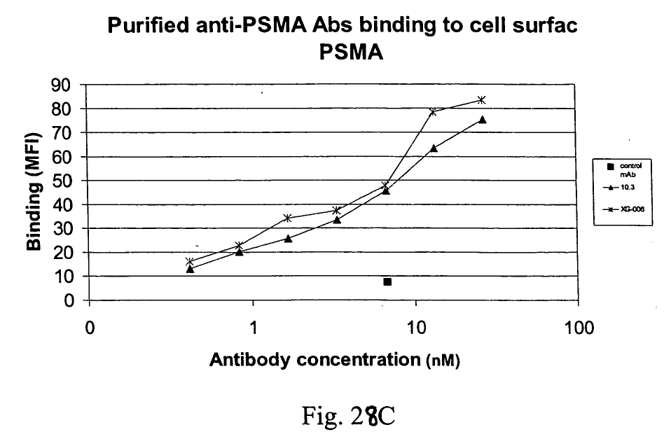

Flow cytometry data (mean fluorescence intensity v. antibody concentration) were transposed and plotted using Excel software (Microsoft, Redmond, Wash.). Results from representative experiments of at least three determinations are depicted in FIGS. 28A-28C. Binding was compared by calculation of 50% effective concentration (EC50) using the Forecast function in Excel. The EC50 value represents the concentration of antibody required for half-maximal binding.

Anti-PSMA mAbs 10.3 (PSMA 10.3) and XG-006 were found to bind to 3T3-PSMA cells and not 3T3 cells (FIG. 28A). Antibody (26 nM) was added to cells, which were analyzed by flow cytometry. Binding to cell-surface PSMA using serial dilutions of anti-PSMA mAb-containing culture supernatants of XG-006, 4.304.1, XG-026 (PTA-4405 and PTA-4406) and 4.49.1 also was demonstrated (FIG. 28B). Binding to cell-surface PSMA using serial dilutions of purified anti-PSMA mAbs XG-006 and 10.3 is represented by FIG. 28C.

### Example 19

### Cytotoxicity of Toxin-Labeled Antibody

PSMA-3T3, LNCaP, and/or C4-2 cells (and control cell lines 3T3 and PC3 that do not express PSMA) were plated at 2,500 cells/100 μL/well in 96-well microplates (Falcon) and were incubated overnight at 37° C. in the presence of 5% CO₂. The media used for PSMA-3T3 (and 3T3) and LNCaP (and C4-2 and PC3) was DMEM or RMPI 1640, respectively, containing 2 mM L-glutamine, 10% FBS, and 1% penicillin-streptomycin. 50 ng (in 50 μL) of Mab-Zap or Hum-ZAP (Advanced Targeting Systems, San Diego, Calif.) in medium was added in each well. Mab-Zap and Hum-Zap are goat anti-mouse IgG antibody or goat anti-human IgG antibody covalently linked to saporin, the most potent of the plant ribosome-inactivating proteins (RIP) from the seeds of the plant Saponaria officinalis. Saporin induces cell death by apoptosis (Bergamaschi, G., Perfetti, V., Tonon, L., Novella, A., Lucotti, C., Danova, M., Glennie, M. J., Merlini, G., Cazzola, M. Saporin, a ribosome-inactivating protein used to prepare immunotoxins, induces cell death via apoptosis. Br J Haematol 93, 789-94. (1996)). The Mab-Zap did not bind to or internalize in cells in the absence of an appropriate primary antibody.

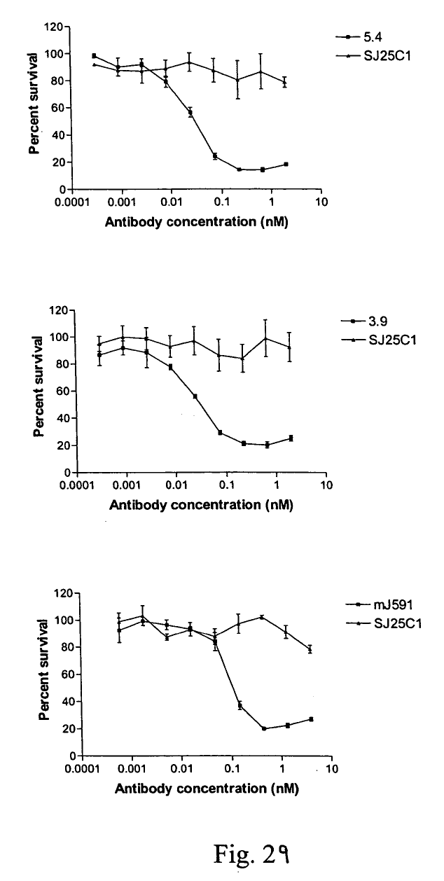

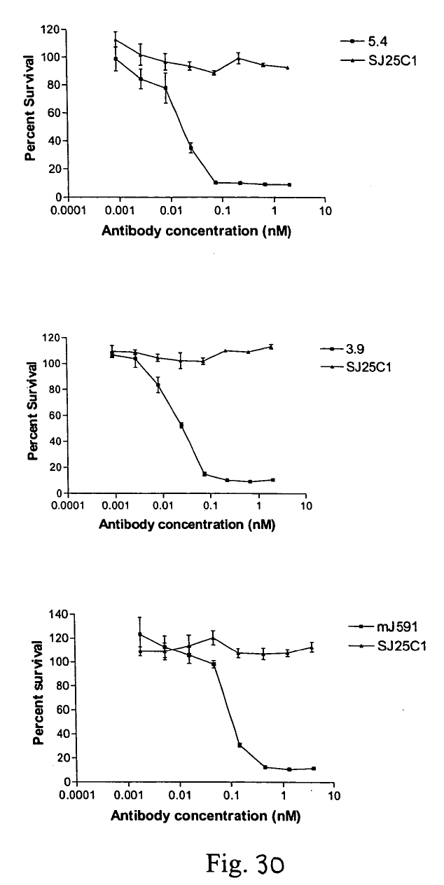

Murine 3.9, 5.4, mJ591 (ATCC# HB-12126) and human 006, 4.40, 4.304 anti-PSMA antibodies (and control IgG antibodies) were added into plates at different concentrations to bring the total volume to 200 μL in triplicate. The plates were kept cold on ice for at least 30 min to maximize Map-Zap or Hum-Zap binding to PSMA antibodies before internalization. The plates were incubated for 2 days and then the medium was changed and incubated for another 2 days. After 4 days incubation, the medium was withdrawn and fresh medium containing 10% Alamar Blue (20 μL, Bioscience, Camarillo, Calif.) was added into each well and incubated for 2 hrs. A CytoFlour plate reader was used to measure fluorescence in 96-well plates at wavelengths of 530 nm excitation and 590 nm emission. Internalization of toxin was mediated by anti-PSMA antibodies. The cell kill is illustrated in FIG. 29 on C4-2 cells and in FIG. 30 on PSMA-3T3 cells.

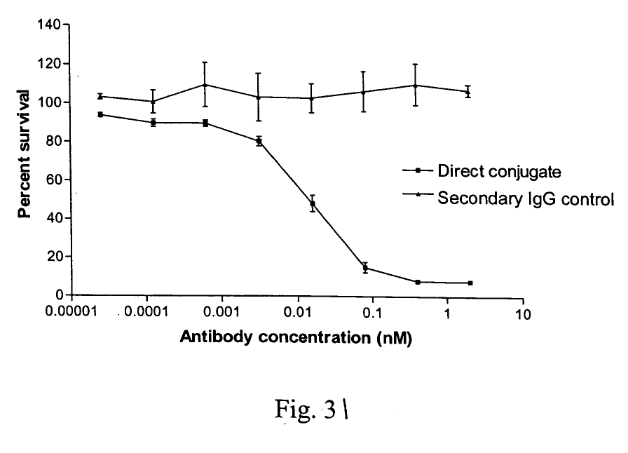

Human 4.304 anti-PSMA antibody was directly conjugated with saporin (Wrenn et al., Brain Res. 740:175-184, 1996), and its cytotoxicity was demonstrated using a similar protocol as described above (see FIG. 31).

### Example 20

### Immunoreactivity

PSMA-3T3, LNCaP and C4-2 were used as PSMA expressing cell lines and 3T3 was used as a control cell line not expressing PSMA. The cells were blocked with 10% goat serum on ice to reduce non-specific binding in this assay.

A small amount (1-5 ng) of labeled mAb was added into a cell pellet of 10 million cells and incubated at 0° C. (on ice) with gentle mixing. After a 1 hour incubation, the cells were collected by centrifugation and the supernatant containing unbound mAb was transferred to a fresh cell pellet for an additional 1 hour incubation at 0° C. Both sets of cells were centrifuged and washed twice with cold PBS. The cell pellets, supernatant and wash fractions were counted for radioactivity. Immunoreactivity is defined as the amount of radioactivity in the cell pellets divided by the total radioactivity in the cell pellets, supernatant and wash fractions. These data are shown below in Table 3.

TABLE 3
Immunoreactivity of ¹¹¹In Radiolabeled
Antibody on PSMA Expressing Cells

| Radiolabeled mAb | Immunoreactivity (%) | Cell line |
|---|---|---|
| ¹¹¹In 4.304 | 92.6 (1.4) | PSMA-3T3 (3T3) |
| | 92.6 | PSMA-3T3 |
| | 91.4 (1.7) | PSMA-3T3 (3T3) |
| | 89.1 | LNCaP |
| | 92.4 | C4-2 |
| Average = | 91.6 ± 1.5 | |
| ¹¹¹In 4.40 | 87.7 (0.5) | PSMA-3T3 (3T3) |
| | 86.8 | PSMA-3T3 |
| | 89.4 (1.5) | PSMA-3T3 (3T3) |
| Average = | 88.0 ± 1.3 | |
| ¹¹¹In mJ591 | 58.5 | PSMA-3T3 |
| | 54.9 (1.1) | PSMA-3T3 (3T3) |
| Average = | 56.7 ± 2.5 | |
| ¹¹¹In 3.9 | 88 | LNCaP |
| | 87 | C4-2 |
| | 89 (2) | PSMA-3T3 (3T3) |
| | 95.3 (0.5) | PSMA-3T3 (3T3) |
| | 88.6 | PSMA-3T3 |
| | 84.8 | C4-2 |
| | 89.3 | PSMA-3T3 |
| Average = | 88.6 ± 3.2 | |

Antibodies 4.40, 4.304 and mJ591 were conjugated to the bifunctional chelate CHX-A″-DTPA and antibody 3.9 was conjugated to C-DOTA.

Immunoreactivity of ²²⁵Ac radiolabeled antibody (026 and 4.40) was also assessed with a methodology similar to that described above for the ¹¹¹In labeled antibodies. ²²⁵Ac was chelated with the bifunctional DOTA at 50° C. for 30 minutes. The chelated ²²⁵Ac was then conjugated to antibodies 026 and 4.40 at 35° C. for 30 minutes. Unconjugated ²²⁵Ac was removed by a PD10 column (Amersham Biosciences, Picataway, N.J.). The immunoreactivity of the radiolabeled antibodies was then determined. The data are presented below in Table 4. In addition to the assessment of the immunoreactivity of these antibodies, the yield of the labeling procedure was also assessed, and these data are also provided below in Table 4.

TABLE 4
Yield and Immunoreactivity of ²²⁵Ac Radiolabeled Antibody

| Antibody | Yield | Immunoreactivity |
|---|---|---|
| 026 | 9.3 +/− 0.8 (n = 2) | 61.3 +/− 1.1 (n = 2) |
| 4.40 | 14.3 +/− 0.6 (n = 2) | 78.1 +/− 0.1 (n = 2) |

### Example 21

### Competitive Binding Assay to Identify Binding Epitopes

To identify whether a given group of mAbs recognize distinct or overlapping epitopes on PSMA, competition binding assays were performed with ¹¹¹In radiolabeled antibodies. 2×10⁵ cells (100 μL) of PSMA-3T3 were plated into 96-well microplates, and antibodies 4.40, 4.304 and mJ591 (100 μL) at different concentrations (series dilution) were added. The cells were incubated at 0° C. for 30 min. 20 μL of In-111 radiolabeled CHX-A″-DTPA antibody constructs were added into each well. After a 2 hour incubation on ice for competition binding, the cells were washed 5 times using cold PBS. The cells containing bound ¹¹¹In antibodies were recovered from microplates into test tubes and counted in a gamma counter.

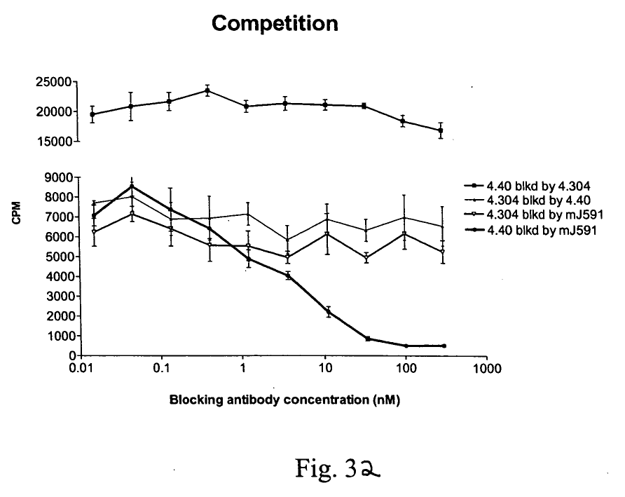

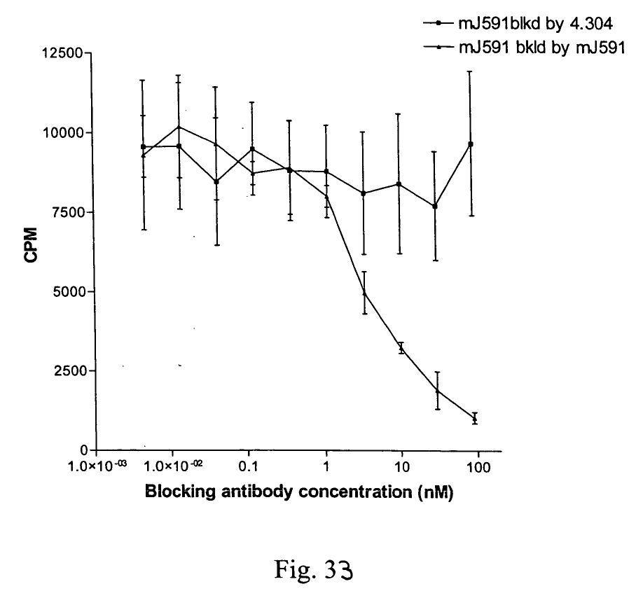

Results detailed in FIG. 32 show that mJ591 blocked ¹¹¹In 4.40 binding to PSMA-3T3 cells and did not block ¹¹¹In 4.304. In addition, 4.40 and 4.304 did not block each other. Unmodified antibodies 4.304 and mJ591 were also used to compete with ¹¹¹In radiolabeled mJ591. Human 4.304 did not compete with ¹¹¹In mJ591 for binding to PSMA-3T3 (FIG. 33).

### Example 22

### Binding Affinity Using Biacore 3000

To determine the kinetics and affinity of the antibodies, the antibodies in crude supernatants, in purified form and in bifunctional chelate modified forms were analyzed using a Biacore 3000 instrument (Biacore Inc., Piscataway, N.J.). Biacore 3000 is a fully automated surface plasmon resonance (SPR)-based biosensor system that is designed to provide real-time kinetic data from assay formats that require no tags or labeling of compounds for biomolecular interactions. It is ideal for screening crude supernatants.

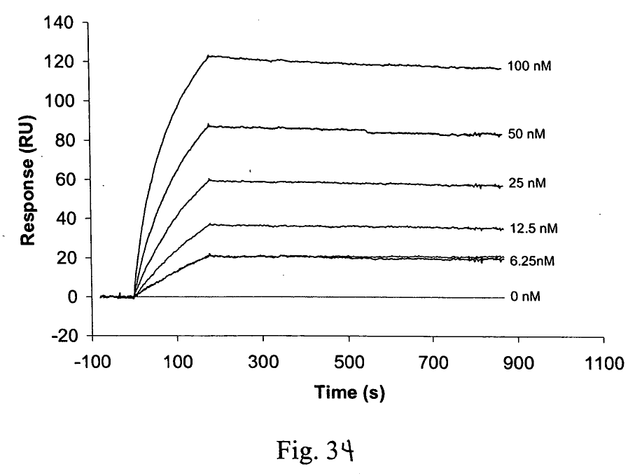

The streptavidin-coated sensor chips (SA chips, Biacore) were used to capture biotinylated anti-human IgG antibody (Sigma, St. Louis, Mo.). The entire sensor chip surface was conditioned with five injections of conditioning solution (1 M NaCl, 50 mM NaOH) and equilibrated with PBS buffer containing 0.005% polysorbate 20. Two to three thousand resonance units (RU) of biotinylated anti-human IgG antibody (Sigma) were immobilized onto the SA chip followed by an injection of regeneration buffer (glycine-HCl, pH 2.2). Antibodies in supernatants were diluted to 2 μg/mL in PBS buffer and captured onto one anti-human IgG flow cell, while isotype-matched control human antibody (Sigma) was similarly captured on a second flow cell. rsPSMA at different concentrations in PBS buffer was flowed over the cells at 30 μL/min for 3 min in an “association phase” followed by a “dissociation phase” for 10 min. SPR was monitored and displayed as a function of time. For each antibody at one concentration, the chip was regenerated and equilibrated. Examples of the analysis of antibody PRGX1-XG-006 in association phase and dissociation phase at different concentrations of rsPSMA from 100 nM to 6.25 nM are shown in FIG. 34. Thermodynamic and kinetic rate constants of binding were calculated using the Biacore Evaluation software. For example, the affinity of XG-006 antibodies in a supernatant to rsPSMA was determined to be 4.92×10⁻¹⁰ M with a Kₐ of 1.3×10⁵ M⁻¹s⁻¹ and a K_{d} of 6.4×10⁻⁵ s⁻¹. Selective data for several human PSMA antibodies in crude supernatant, purified form, and modified with bifunctional chelate is listed in Table 5 for comparison.

Binding activity of ¹¹¹In radiolabeled antibodies was determined by Scatchard analysis of binding data obtained using PSMA-expressing cells (LNCaP, C4-2, PSMA-3T3 and parental 3T3 as a control). The experimental procedures and methods of data analysis have been described previously (Scheinberg, D. A. et al. Leukemia 3: 440-445 (1991).

TABLE 5
Kinetic Rate Constants of Antibodies in Crude Supernatant,
Purified, Bifunctional Chelate Modified Forms along with KD
Determined Using¹¹¹In Radiolabeled Scatchard Analysis

| | Ka | | | |
|---|---|---|---|---|
| | (M⁻¹, | Kd | KD | |
| Antibodies | s⁻¹) | (s⁻¹) | (M⁻¹) | Avg KD |
| 006 Supernatant | 1.30E+05 | 6.40E−05 | 4.92E−10 | 4.92E−10 |
| Purified 006-1 | 2.94E+05 | 1.37E−04 | 4.66E−10 | |
| Purified 006-2 | 2.26E+05 | 1.27E−04 | 5.62E−10 | 5.14E−10 |
| 4.40 Supernatant | 2.10E+05 | 1.25E−04 | 5.95E−10 | 5.95E−10 |
| Purified 4.40-1 | 2.54E+05 | 1.52E−04 | 5.98E−10 | |
| Purified 4.40-2 | 2.43E+05 | 2.37E−04 | 9.75E−10 | 7.87E−10 |
| CHX-4.40-1 | 2.57E+05 | 1.60E−04 | 6.23E−10 | |
| CHX-4.40-2 | 2.47E+05 | 1.55E−04 | 6.28E−10 | 6.25E−10 |
| IN-111CHX-4.40-1 | | | 4.44E−09 | |
| IN-111CHX-4.40-2 | | | 4.95E−09 | 4.70E−09 |
| 4.304 Supernatant | 1.40E+05 | 1.25E−04 | 8.93E−10 | 8.93E−10 |
| Purified 4.304-1 | 8.31E+04 | 1.20E−04 | 1.44E−09 | |
| Purified 4.304-2 | 1.06E+05 | 6.33E−05 | 5.97E−10 | 1.02E−09 |
| CHX-4.304-1 | 6.19E+04 | 1.21E−04 | 1.95E−09 | |
| CHX-4.304-2 | 6.79E+04 | 1.49E−04 | 2.19E−09 | 2.07E−09 |
| IN-111CHX-4.304-1 | | | 9.63E−09 | |
| IN-111CHX-4.304-2 | | | 5.97E−09 | 7.80E−09 |
| 10.3 Supernatant | 1.90E+05 | 3.63E−04 | 1.91E−09 | 1.91E−09 |
| Purified 10.3-1 | 3.28E+05 | 6.32E−05 | 1.93E−10 | |
| Purified 10.3-2 | 2.96E+05 | 6.43E−05 | 2.17E−10 | 2.05E−10 |

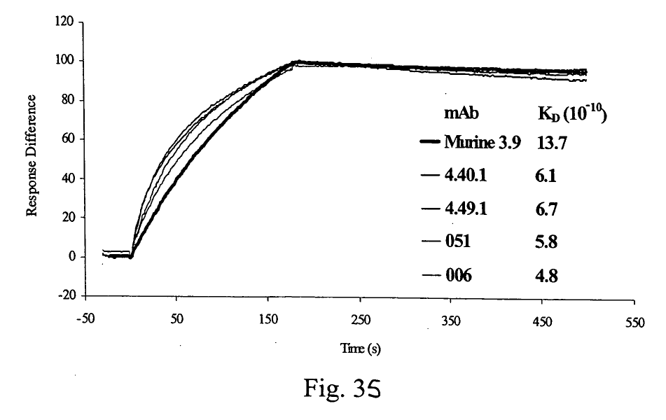

A comparison of the fully human antibodies 4.40.1, 4.49.1, 051 and 006 and the murine antibody 3.9 was performed by Biacore. For each antibody for comparison, response was normalized to 100 RU. The graph of time vs. response difference for these antibodies is given in FIG. 35. The binding affinities for these antibodies were determined to be 6.1, 6.7, 5.8, 4.8 and 13.7×10⁻¹⁰M, respectively.

### Example 23

### Characterization of Cell Lines for In Vitro and In Vivo Studies

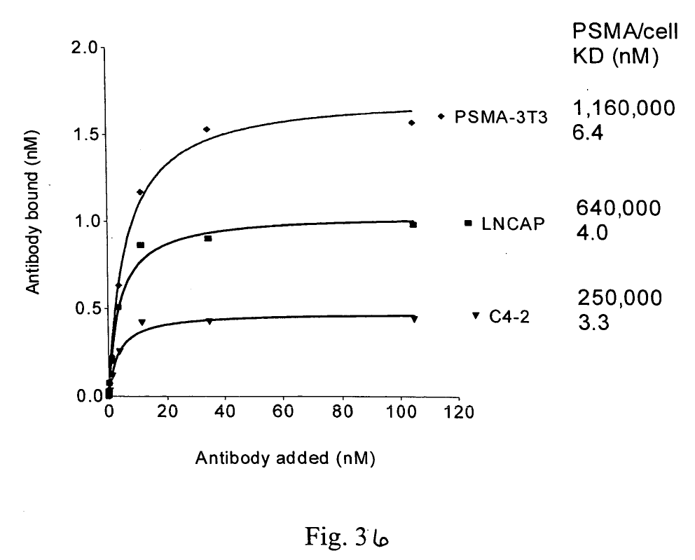

Results from a Scatchard analysis using ¹¹¹In labeled anti-PSMA antibody 3.9 are represented in FIG. 36. Transfected murine 3T3 cells express >1 million copies of PSMA per cell, LNCAP cells (androgen dependent human prostate cancer cell line) express 0.64 million copies, while C4-2 cells (androgen independent) express 0.25 million copies per cell. The affinity of 3.9 for cell surface PSMA is 6.4 μM for PSMA-3T3, 4.0 nM for LNCAP and 3.3 nM for C4-2 (4.6 nM is the average of these data).

A summary of the analyses of crude supernatants for the human anti-PSMA antibodies is given in Table 6 below.

TABLE 6
Characterization of Anti-PSMA Monoclonal Antibodies

| | | | Binding to 3T3- | | | | | | | |
|---|---|---|---|---|---|---|---|---|---|---|
| | Ab Conc | | PSMA (FACS) | | | | | Biacore studies | | |
| | (μg/mL) | | | AVG | | | Anti- | KD, | Ka, | Kd, |
| | | Lysate | PGNX | Max | AVG | C4.2 | PSMA | M-1 | M-1s-1 | s-1 |
| Supernatant | PGNX | EIA | FACS | binding | EC50 | FACS | Western | (×10⁻¹⁰) | (×10⁵) | (× 10 − 5) |
| PRGX1-XG1- | 4.7 | ND¹ | ND | 148 | 2.4 | ND | Conf.² | 2.0 | 1.5 | 2.9 |
| 026 | | | | | | | | | | |
| 4.4.1 | 4.7 | 0.08 | 7 | 8 | ND | 5.2 | Conf. | 4.2 | 2.3 | 9.7 |
| PRGX1-XG1- | 1.8 | 0.39 | 114 | 183 | 3.4 | 9.5 | Conf. | 4.8 | 1.3 | 6.4 |
| 006 | | | | | | | | | | |
| PRGX1-XG1- | 3.5 | 0.48 | 83 | 202 | 2.0 | 9.9 | Conf. | 5.8 | 1.4 | 8.2 |
| 051 | | | | | | | | | | |
| 4.40.1 | 4.3 | 0.33 | 53 | 163 | 2.3 | 10.8 | Conf. | 6.1 | 2.1 | 12.5 |
| 4.49.1 | 2.6 | 0.36 | 362 | 162 | 0.9 | 16.2 | Conf. | 6.7 | 3.1 | 20.7 |
| 4.292.1 | 2.7 | 0.18 | 75 | 195 | 6.0 | 9.2 | Conf. | 6.8 | 1.2 | 8.5 |
| 4.304.1 | 4.1 | 0.39 | 92 | 184 | 9.1 | 8.4 | Conf. | 8.7 | 1.4 | 12.5 |
| 4.232.1 | 2.4 | 0.49 | 97 | 138 | 2.7 | 6.0 | Linear³ | 9.4 | 1.5 | 13.8 |
| 4.153.1 | 5.9 | 0.29 | 279 | 182 | 5.3 | 14.8 | Conf. | 9.5 | 1.2 | 11.8 |
| 4.333.1 | 2.9 | 0.18 | 82 | 168 | 3.1 | 6.6 | Conf. | 11 | 0.7 | 8.5 |
| PRGX1-XG1- | 3.9 | 0.45 | 392 | 227 | 6.0 | 12.4 | Conf. | 16 | 0.6 | 10.4 |
| 077 | | | | | | | | | | |
| 10.3 | 8.5 | 1.06 | ND | ND | ND | ND | ND | 19 | 1.9 | 36.4 |
| pure 10.3 | | 0.44 | 130 | 181 | 7.5 | ND | Conf. | ND | | |
| | | | | | | 4.7 | | | | |
| 4.22.1 | 2.8 | 0.08 | 7 | ND | ND | 4.7 | ND | 20 | 1.7 | 33 |
| 4.248.1 | 3.5 | 0.37 | 7 | ND | ND | 4.1 | Conf. | 27 | 1.0 | 28 |
| 4.54.1 | 10 | 0.14 | 267 | 162 | 3.9 | 13.6 | ND | 30 | 1.9 | 56 |
| 4.7.1 | 5 | 0.23 | 156 | 141 | 1.6 | 10.2 | Conf. | 32 | 1.7 | 56 |
| 4.78.1 | 5.3 | 0.00 | 205 | 118 | 1.0 | 7.9 | Conf. | 53 | 2.4 | 125 |
| 4.48.1 | 4.9 | 0.06 | 14 | ND | ND | 7.7 | ND | 62 | 0.9 | 59 |
| 4.209.1 | 3.5 | 0.22 | 60 | ND | ND | 6.7 | ND | 142 | 0.9 | 125 |
| 4.177.1 | 1.1 | 0.15 | 236 | 174 | 2.4 | 10.6 | ND | 155 | 0.6 | 93 |
| 4.152.1 | 3.4 | 0.38 | 81 | 85 | 4.0 | 7.5 | ND | 163 | 0.8 | 126 |
| 4.28.1 | 4.2 | 0.04 | 112 | 155 | 4.2 | 11.3 | ND | 167 | 1.2 | 192 |
| 4.16.1 | 5.3 | 0.00 | 8 | ND | ND | 7.8 | ND | 177 | 1.8 | 313 |
| 4.360.1 | 1.5 | 0.02 | 112 | 130 | 2.2 | 7.9 | ND | 197 | 1.0 | 201 |
| 4.288.1 | 15.4 | 0.02 | 67 | 141 | 4.1 | 6.5 | ND | 198 | 1.3 | 257 |
| 4.219.2 | 0.5 | 0.34 | 69 | ND | ND | 5.9 | ND | ND | | |
| PRGX1-XG1- | 6.5 | ND | ND | 71 | 7.9 | ND | ND | No Binding | | |
| 069 | | | | | | | | | | |
| Murine 3.9 | | | | | | | | 13.7 | 0.7 | 9.7 |
| Control | | | | | | | | 6.34 | 2.24 | 14.2 |

¹ND = not determined
²conf. = conformational epitope
³linear = linear epitope

### Example 24

### Cytotoxicity of Radiolabeled Antibody

The in vitro cytotoxicity of ²²⁵Ac labeled anti-PSMA antibody (4.40 and 026) was determined using methodology similar to that used in Example 19. Prostate cancer cells (100 μL of C4-2, LNCaP, and PC3 cells at a concentration of 2×10⁴ cells/mL) were placed into separate wells of a 96 well microplate. For tests with the 026 antibody, C4-2 and PC3 cells were placed into separate wells of a 96 well microplate. After overnight incubation, the cells were treated with ²²⁵Ac labeled human anti-PSMA antibody at different concentrations for over 4 days. Cell cytotoxicity was quantified using Alamar Blue (Biosource International, Camarillo, Calif.).

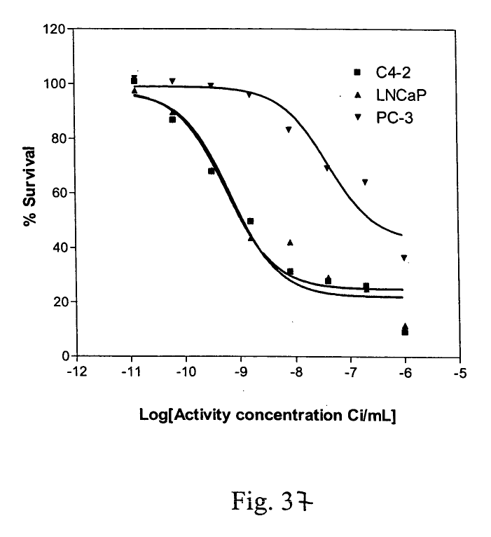

FIG. 37 shows a plot of cell survival vs. ²²⁵Ac activity concentration using ²²⁵Ac labeled 4.40 antibody. The EC50 for PSMA expressing cells (C4-2 and LNCaP) was <2 nCi/mL. However, the EC50 was 420 nCi/mL for PC3 cells, which do not express PSMA on the cell surface. Therefore, the ²²⁵Ac labeled human anti-PSMA 4.40 antibody shows >200-fold selectivity in killing PSMA expressing prostate cancer cells (C4-2 and LNCaP) vs. control cells (PC3).

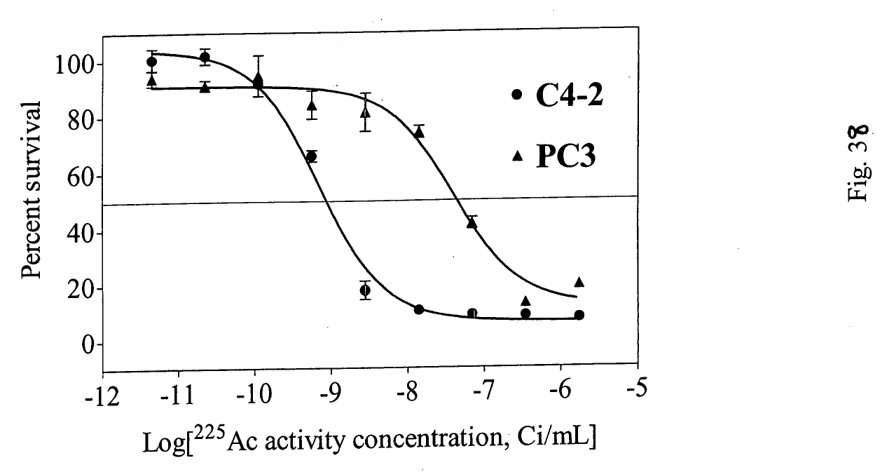

FIG. 38 shows a plot of cell survival vs. ²²⁵Ac activity concentration using ²²⁵Ac labeled 026 antibody. The ²²⁵Ac labeled human anti-PSMA 026 antibody shows >50-fold selectivity in killing PSMA expressing prostate cancer cells (C4-2) vs. control cells (PC3).

### Example 25

### Cytotoxicity of ²²⁵Ac Labeled Antibody vs. Control Antibody

The in vitro cytotoxicity of ²²⁵Ac labeled anti-PSMA antibody was determined using methodology similar to that used in Example 19 and Example 24 above. Human prostate cancer cells (100 μL of C4-2 and LNCaP cells at a concentration of 2×10⁴ cells/mL) were placed into separate wells of a 96 well microplate. After overnight incubation, the cells were treated with ²²⁵Ac labeled human anti-PSMA 026 antibody at different concentrations for 4 days. Cell cytotoxicity was quantified using Alamar Blue (Biosource International, Camarillo, Calif.). Human IgG (HuIgG) was used as a control. The cytotoxicity of an anti-PSMA mAb 026 “2 hour wash” was also determined. A 2 hour wash means that the cells were incubated with ²²⁵Ac labeled antibody for 2 hours. After 2 hours, the media was removed and fresh media was added for the 4 day incubation.

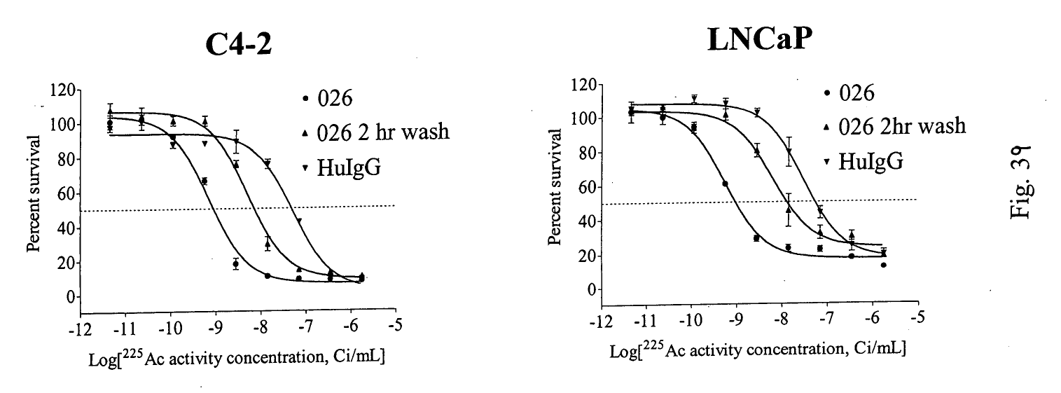

FIG. 39 shows a plot of cell survival vs. the ²²⁵Ac activity concentration for both C4-2 and LNCaP cells using radiolabeled mAb 026, mAb 026 2 hour wash and HuIgG. ²²⁵Ac labeled mAb 026 showed an IC50 of <1 nCi/mL. Therefore, the ²²⁵Ac labeled human anti-PSMA 026 antibody showed >50-fold selectivity in killing the prostate cancer cells vs. the control antibody.

### Example 26

### Cytotoxicity of ²²⁵Ac Labeled Antibody vs. Control Antibody Evaluated by ³H Thymidine Incorporation

Human prostate cancer cells (C4-2) in a 96 microplate were treated with ²²⁵Ac labeled mAbs at different concentrations for 4 days. Cell survival was assessed using ³H thymidine incorporation (Nikula, T.K, et al. J. Nucl. Med. 40: 166-176, 1999).

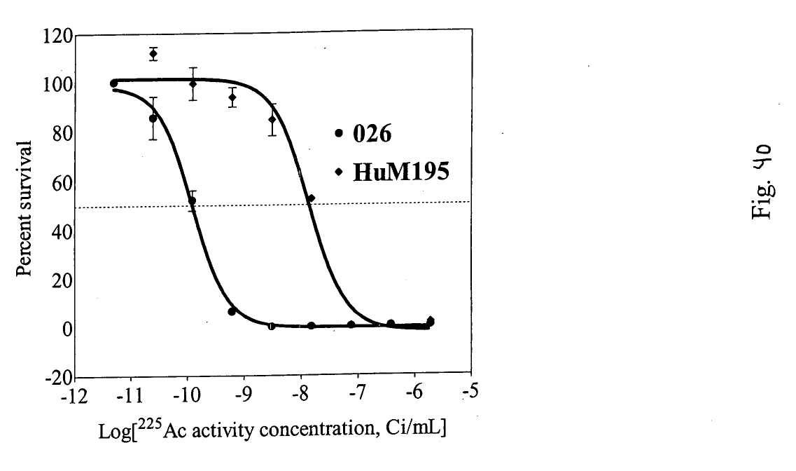

FIG. 40 shows a plot of cell survival vs. the ²²⁵Ac activity concentration for C4-2 cells using radiolabeled mAb 026 and control mAb (HuM195). The IC50 was 0.12 nCi/mL using ²²⁵Ac labeled 026 vs. 13 nCi/ml with the control mAb (HuM195). The radiolabeled 026 antibody, therefore, showed >100-fold selectivity in killing the PSMA expressing C4-2 cells vs. the control antibody.

### Example 27

### In Vivo Radioimmunotherapy with ¹⁷⁷Lu Labeled Antibodies

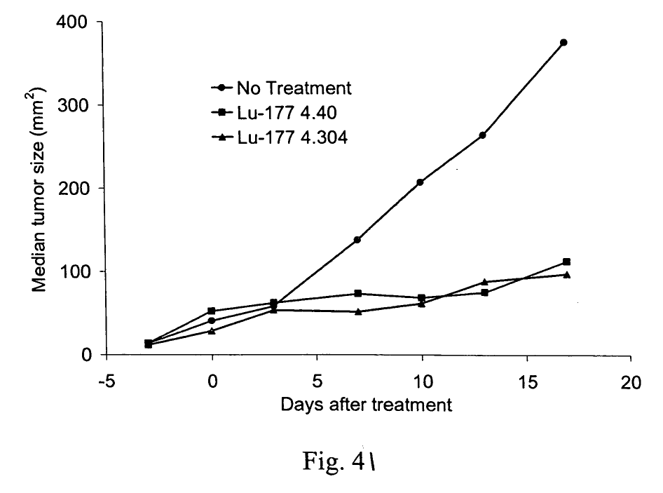

Athymic nude mice from the National Cancer Institute were implanted subcutaneously with 2×10⁶ PSMA-3T3 cells. After measurable tumors appeared at day 7 post implantation, the mice were treated by injection with either a single 250 μCi dose human anti-PSMA antibody 4.40 or 4.304 labeled with ¹⁷⁷Lu (University of Missouri Research Reactor), or were injected with buffer only as control. The tumor size of individual animals was measured using an electronic caliper. FIG. 41 shows a plot of the median tumor size in each group over time. Tumor growths were substantially reduced in ¹⁷⁷Lu antibody treated groups compared to the control group.

### Example 28

### In Vivo Biodistribution Study with ¹⁷⁷Lu Labeled Antibodies

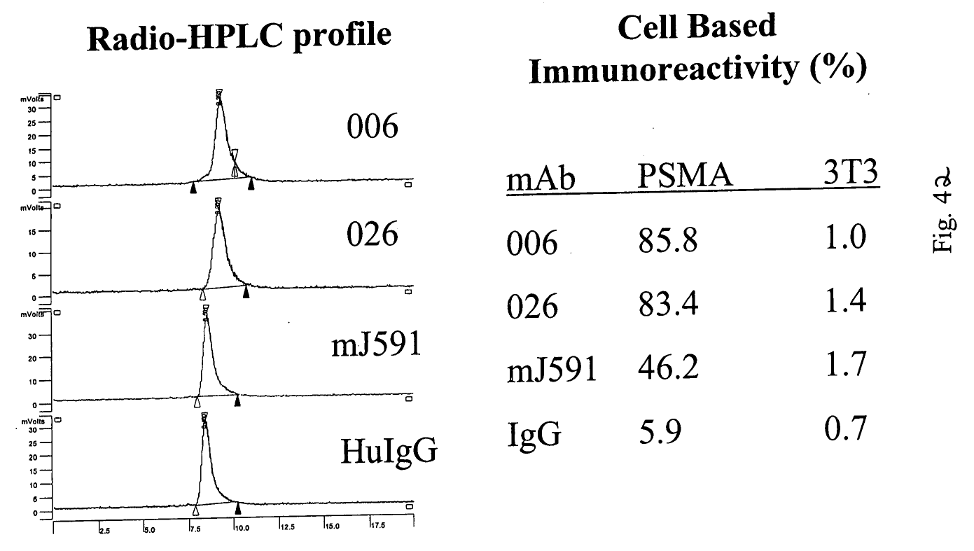

Athymic nude mice from the National Cancer Institute (male, approximately 6 weeks old) were injected subcutaneously with 4×10⁶ PSMA-3T3 cells and 2.8×10⁶ 3T3 cells in 0.2 mL in the right and left flank of each animal, respectively. Anti-PSMA antibodies 006, 026, mJ591 and HuIgG (control) modified with CHX-A″-DTPA were labeled with ¹⁷⁷Lu. FIG. 42 shows the radio-HPLC profile of the radiolabeled antibodies as well as the cell-based immunoreactivity performed as quality control. On day 6 after tumor implantation, ¹⁷⁷Lu labeled antibodies (10 μCi and 1 μg in 0.15 mL) were injected retro-orbitally. The animals were randomized before antibody injection. Mice (30 per antibody, 5 per time point) were sacrificed at different times (days 0.17, 1, 2, 4, 7 and 12). Tumors and individual organs (PSMA+ tumor, PSMA− tumor, blood, liver, kidneys, spleen, lungs, heart, bone, muscle, carcass) were taken and weighed. Activity in each organ along with standards prepared from injection solutions were counted using a multi-channel gamma counter.

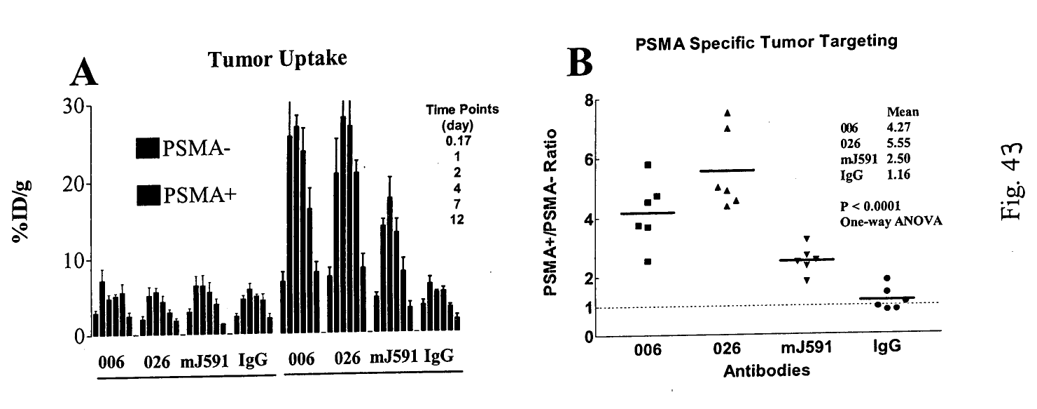

Results of this study show that ¹⁷⁷Lu labeled antibodies specifically bound to tumors expressing PSMA in vivo in the animal model. The percent injected dose per gram of tissue (% ID/g) was calculated and plotted over time for the different antibodies in the PSMA+ and PSMA− tumors (FIG. 43A). PSMA specific tumor targeting (ratio of PSMA+/PSMA− tumor uptake) is provided in FIG. 43B.

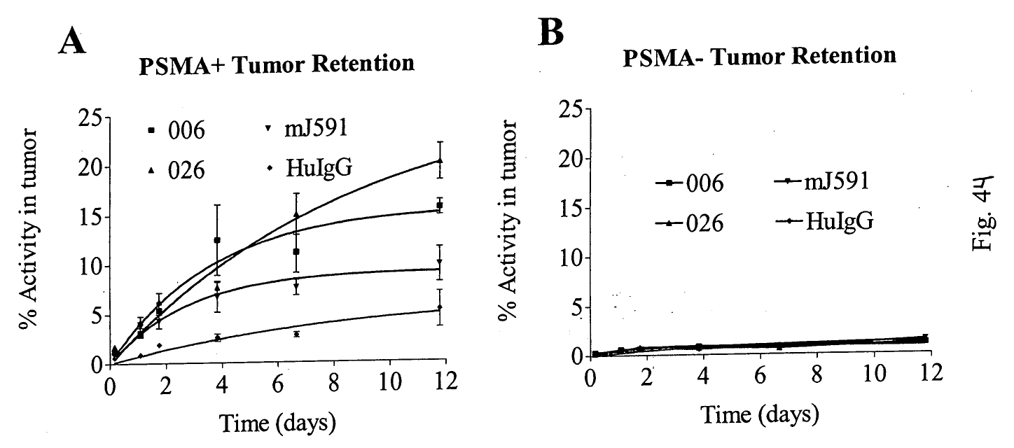

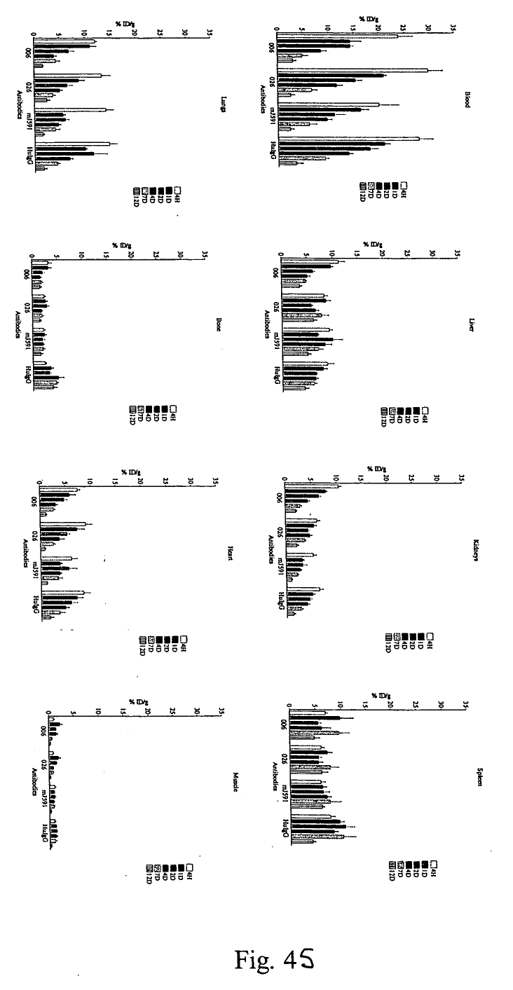

FIG. 44 shows the percent activity in the tumors with the various radiolabeled antibodies (006, 026, mJ591 and HuIgG) over time (% tumor retention vs. total body retention). The data again illustrate the specificity by which the radiolabeled antibodies target the PSMA expressing tumors. FIG. 44A shows the activity over time in the PSMA+ tumors while FIG. 44B shows the percent activity over time in the PSMA− tumors for the different antibodies. FIG. 45 shows the data for normal organ (blood, liver, kidneys, spleen, lungs, bone, heart and muscle) uptake (% ID/g) plotted over time.

### Example 29

### In Vivo Therapeutic Efficacy of ¹⁷⁷Lu Radiolabeled Antibodies

Athymic nude mice from the National Cancer Institute (male, approximately 6 weeks old) were injected subcutaneously with 4×10⁶ PSMA-3T3 cells and 2.8×10⁶ 3T3 cells in 0.2 mL in the right and left flank of each animal, respectively. ¹⁷⁷Lu labeled mAb 026 (0 μCi, n=5; 300 μCi and 10 μg, n=9; and 400 μCi and 13.5 μg, n=5) were injected into the mice on day 6 after tumor implantation. Animals were weighed and tumors were measured over time. Tumor size (mm³) was calculated using the formula: length×(width)²/2. Mice were sacrificed if tumor size reached 1000 mm³. Animal survival was also assessed, and the Kaplan-Meier plot was created.

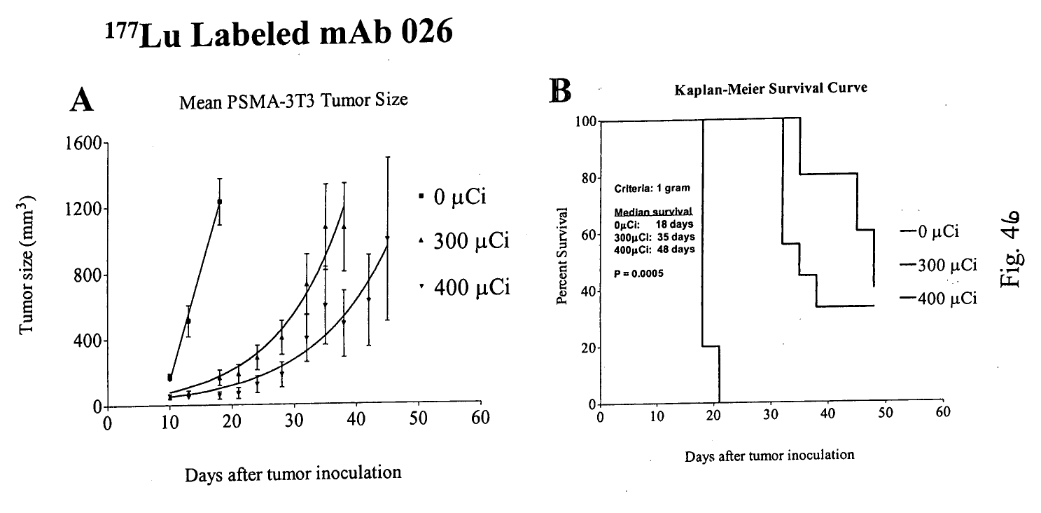

The results of the study show that treatment decreased tumor size and increased survival in the mice. FIG. 46A shows the tumor size in the mice treated with the radiolabeled antibodies (¹⁷⁷Lu labeled mAb 026) at all three dose levels. The mice treated with 300 μCi and 400 μCi had consistently smaller tumors than the mice in the control group (0 μCi). FIG. 46B shows that the mice treated with 300 μCi and 400 μCi had increased survival relative to the control mice. Median survival was increased by 2.4-fold in mice treated with 300 μCi and 3.5-fold in mice treated with 400 μCi using time after treatment. Treatment with 400 μCi was found to be non-toxic. Additionally, at the end of the experiment (48 days after tumor implantation), one animal from each treated group remained PSMA-3T3 tumor free but had large 3T3 tumors.

### Example 30

### Binding of Antibodies to rsPSMA Dimer and Monomer

A Biacore 3000 instrument was used to monitor, in real time, binding of rsPSMA dimer and monomer to anti-PSMA mAbs. Antibodies were immobilized at approximately 10,000 resonance units to CM5 sensor chips according to the manufacturer's instructions for amine coupling (Biacore, Inc., Piscataway, N.J.). A reference surface of isotype-matched antibody of irrelevant specificity was used as a background control. Binding experiments were performed at 25° C. in PBS buffer with 0.005% [vol/vol] Surfactant P20. Purified rsPSMA dimer (50 nM) or monomer (100 nM) was passed over control and test flow cells at a flow rate of 5 μL/min. The sensor surface was regenerated with two pulses of 20 nM HCl.

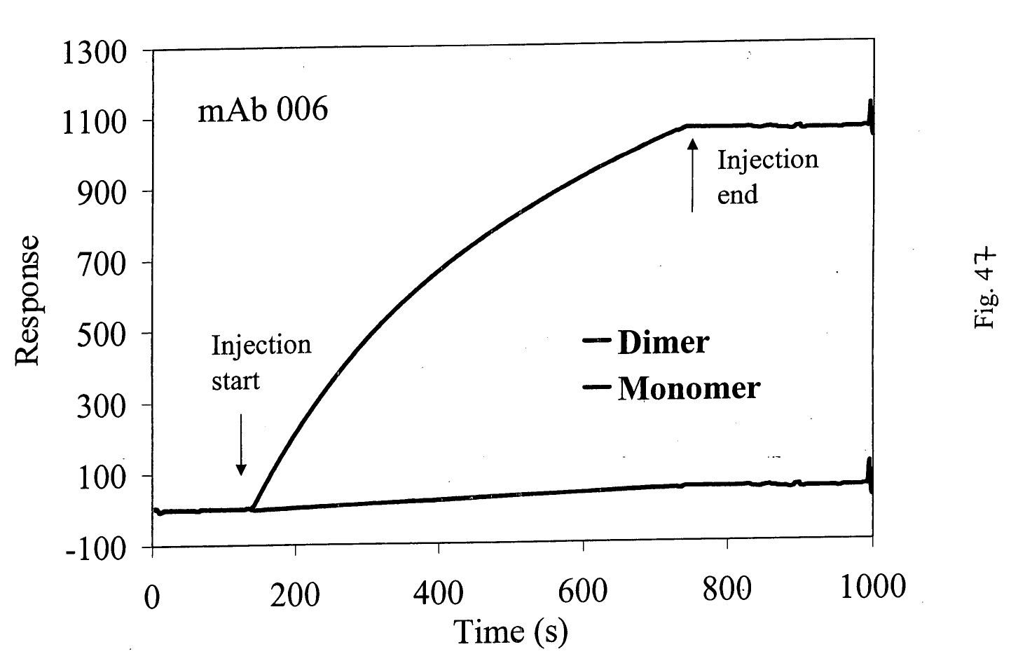

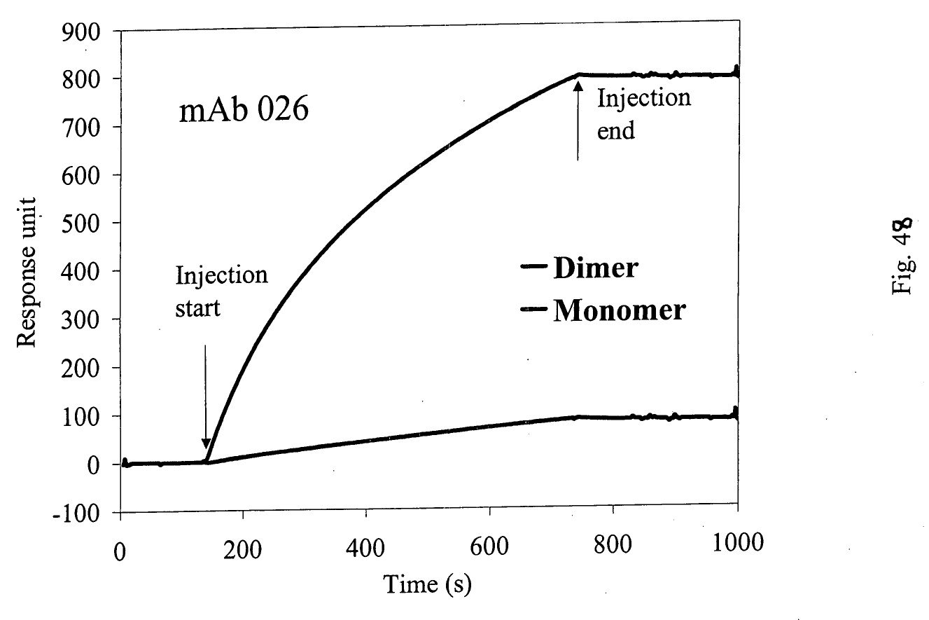

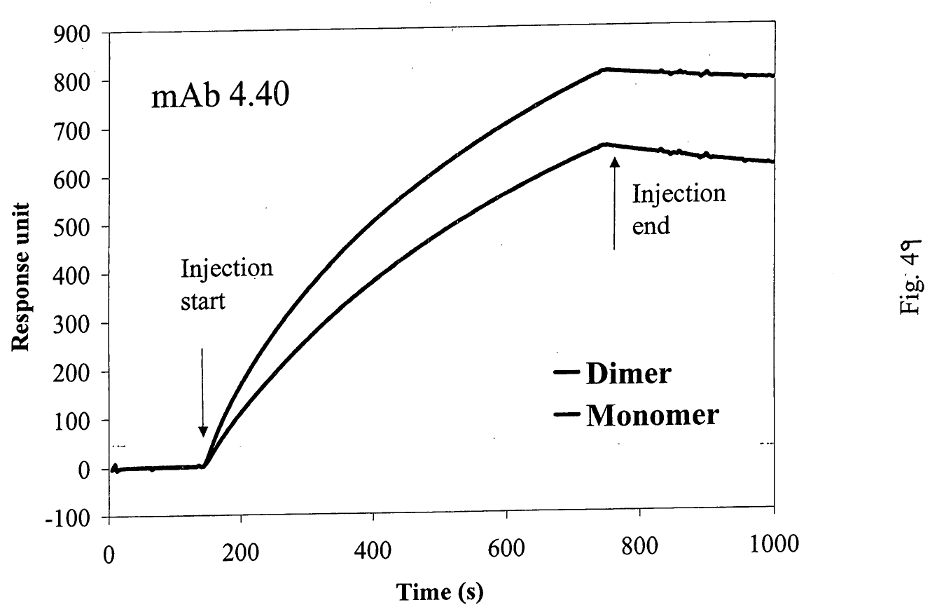

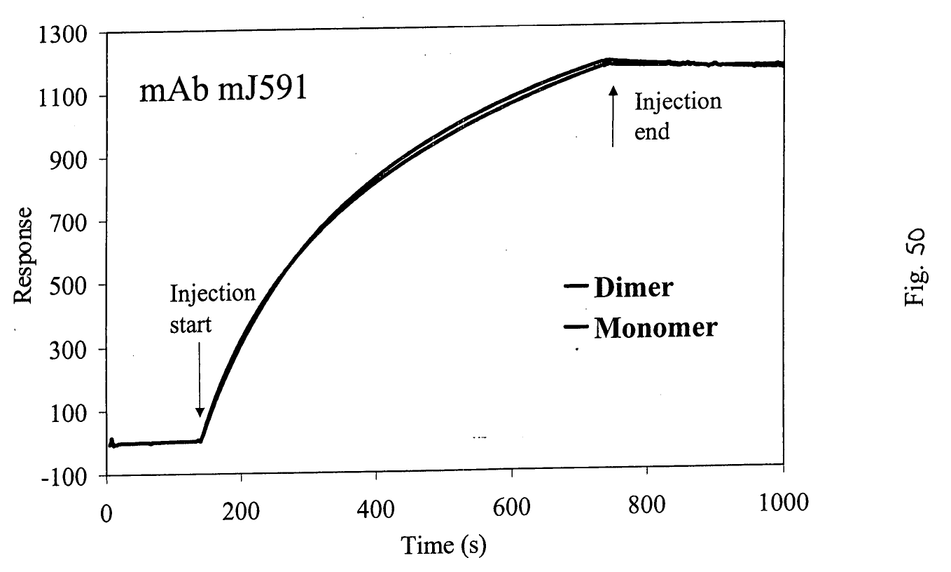

FIGS. 47 and 48, respectively, show that anti-PSMA mAbs 006 and 026 bind preferentially to the rsPSMA dimer rather than the rsPSMA monomer. Anti-PSMA antibodies 4.40 and mJ591, however, were shown to bind both the rsPSMA dimer and monomer at significant levels (FIGS. 49 and 50, respectively). This study illustrates that anti-PSMA mAbs 006 and 026 are PSMA dimer-specific antibodies and bind dimer-specific epitopes on PSMA. The results also indicate that the native conformation of PSMA is a homodimer, and that the monomer possesses a partially denatured conformation or exposes epitopes located at the dimer surface and/or dimer interface that are not accessible in the dimer.

### Example 31

### Immunization with rsPSMA Dimer Preparations

Immunization

BALB/c mice were immunized by subcutaneous injection at days 0, 7, 14, and 42 with either 5 μg clinical rsPSMA lot # 4019-C001 (75% dimer/25% monomer) or 5 μg rsPSMA batch # TD045-003 run 1/peak 2 (100% monomer) on alum (250 μg per dose, Sigma) or adjuvanted with 50 μg alhydrogel per dose. Serum was drawn 10 days after the fourth immunization and analyzed by enzyme-linked immunoassay (EFIA) and flow cytometry.

EIA

rsPSMA lot # 4019-C001 or rsPSMA batch # TD045-003 run 1/peak 2 was passively adsorbed to 96-well microtiter plates. Remaining binding sites on the plate were blocked with a PBS/Casein/Tween 20 buffer. Serially diluted mouse serum or controls were added and bound antibody was detected using a goat anti-mouse IgG antibody conjugated to alkaline phosphatase. The EIA was developed with the substrate pNPP which produces a color change that is directly proportional to the amount of anti-PSMA antibody bound. Absorbance was read at 405 nm with a correction of 620 nm. Antibody titer was defined as the highest dilution of mouse serum yielding a blank corrected absorbance of 0.1. Immune mouse serum with a known anti-PSMA titer or normal mouse serum with no anti-PSMA reactivity was used as controls.

Flow Cytometry Analysis

PSMA-3T3 cells were incubated with 200 μL of immune serum at a dilution of 1/50 in PBS with 0.1% sodium azide on ice for 30 minutes. Immune mouse serum with known anti-PSMA titer or normal mouse serum with no anti-PSMA reactivity was used as controls. The cells were washed twice with PBS with 0.1% sodium azide and incubated for 30 minutes on ice with FITC-conjugated goat anti-mouse IgG. Cells were washed once, resuspended in PBS with 0.1% sodium azide and subjected to flow cytometric analysis on FACScaliber (Becton Dickinson).

Results

5/5 mice immunized with rsPSMA lot # 4019-C001 showed an anti-PSMA antibody response by EIA. Antibody titer was similar for assay plates coated with rsPSMA lot # 4019-C001 (75% dimer/25% monomer) and assay plates coated with rsPSMA batch # TD045-003 run 1/peak 2 (100% monomer). Median response for the group was 1/6400.

4/5 mice immunized with rsPSMA batch # TD045-003 run 1/peak 2 showed an anti-PSMA antibody response by EIA. One mouse was negative. Antibody titer was similar for assay plates coated with rsPSMA lot # 4019-C001 (75% dimer/25% monomer) and assay plates coated with rsPSMA batch # TD045-003 run 1/peak 2 (100% monomer). Median response for the group was 1/6400.

The results of the EIA analysis are provided in Table 7.

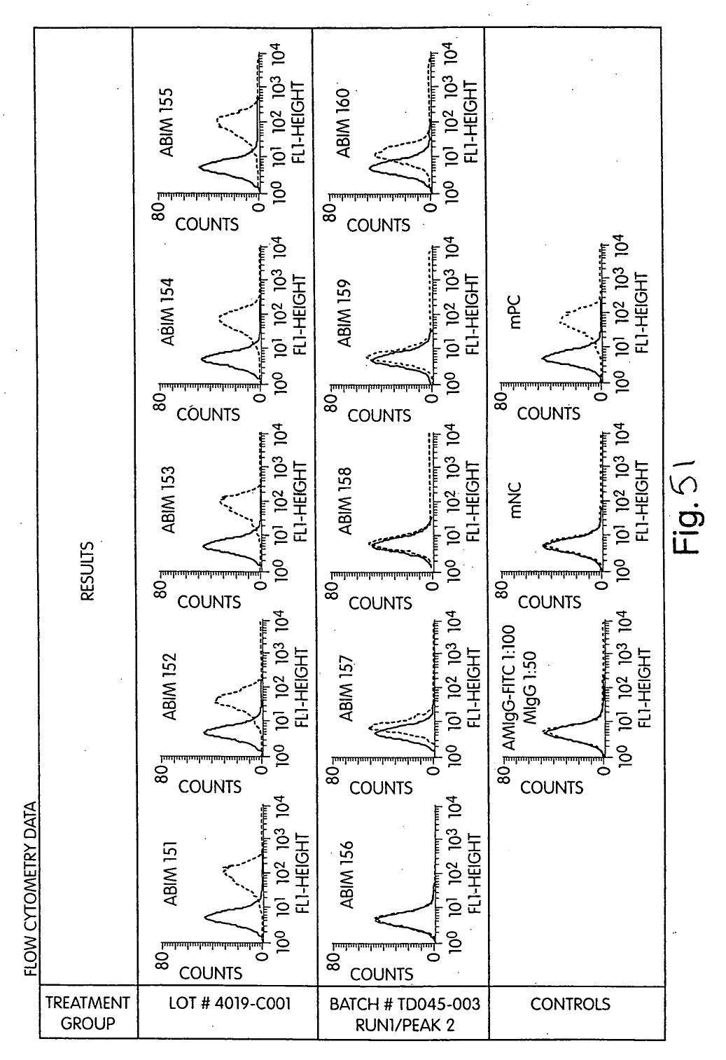

The results of the flow cytometry analysis are provided in FIG. 51.

TABLE 7
Specificity of the Anti-PSMA Antibody Response in Mice Vaccinated
4 Times with rsPSMA 5 μg/dose and 50 μg/dose Alhydrogel

| | | | EIA Titer | Median |
|---|---|---|---|---|
| | | EIA Titer | vs. Batch | RFI vs. |
| Mouse | | vs. Lot | TD045-003 | PSMA-3T3 |
| ID # | Immunogen | 4019-C001 | run 1/peak 2 | cells |
| ABIM151 | 4019-C001 | 1/3200 | 1/3200 | 84 |
| | Dimer | | | |
| ABIM152 | 4019-C001 | 1/3200 | 1/3200 | 41 |
| | Dimer | | | |
| ABIM153 | 4019-C001 | 1/25600 | 1/25600 | 76 |
| | Dimer | | | |
| ABIM154 | 4019-C001 | 1/12800 | 1/12800 | 63 |
| | Dimer | | | |
| ABIM155 | 4019-C001 | 1/6400 | 1/6400 | 74 |
| | Dimer | | | |
| ABIM156 | Monomer | 1/1600 | 1/1600 | 5 |
| ABIM157 | Monomer | 1/6400 | 1/12800 | 8 |
| ABIM158 | Monomer | 0 | 0 | 6 |
| ABIM159 | Monomer | 1/6400 | 1/6400 | 6 |
| ABIM160 | Monomer | 1/6400 | 1/6400 | 12 |

When tested by ELISA, sera from both monomer and dimer immunized animals showed similar levels of anti-PSMA antibodies, indicating that each protein was immunogenic when formulated on alum. For dimer immunized animals, the median endpoint titers were 1/6,400 (range 1/3,200 to 1/12,800) regardless of whether rsPSMA monomer or dimer was used as the coating antigen. Similarly, monomer-immunized animals had median endpoint titers of 1/6,400 in both assay formats, although the range varied depending on whether the monomer (range <1/400 to 1/12,800) or dimer (range <1/400 to 1/6,400) was used for coating.

However, a difference between sera was observed with cell-based flow cytometry (FIG. 51). Anti-PSMA antibody in the serum of mice immunized with a dimer preparation of rsPSMA (lot # 4019-C001) showed strong binding to PSMA-3T3 cells. Anti-PSMA antibody in the serum of mice immunized with a 100% monomer preparation of rsPSMA (batch # TD045-003 run 1/peak 2) showed no binding to PSMA-3T3 cells.

Each dimer immunized animal elicited high-titered antibodies to PSMA-3T3 cells (median mean fluorescence intensity (MFI)=74, range 41-84), but such antibodies were very weak to absent in monomer-immunized animals (median MFI=6, range 5-12). The level of binding observed for monomer immunized animals was comparable to that for naïve animals. Similar background levels of binding to parental 3T3 cells were observed for all sera (median MFI=6 in all cases).

An identical pattern of reactivity was observed with human prostate cancer cell lines. Consistent, high-level reactivity with PSMA-expressing C4-2 cells was observed for sera from dimer immunized animals (median MFI=28.0, range 23.1-28.8) but not monomer immunized (median MFI=12.8, range 11.2-14.5) or control animals (median MFI=12.3, range 8.6-16.0). Background levels of binding to PSMA-negative PC-3 cells were observed for all sera (median MFI=7 in all cases).

Thus, while it is possible to elicit the production of antibodies that recognize native PSMA using monomeric forms of the PSMA protein or fragments thereof, these results speak to the relative efficiency of eliciting an immune response to native PSMA using dimeric forms of PSMA protein. Additionally, flow cytometry but not ELISA was able to reveal the differences in the humoral immune responses elicited by monomeric and dimeric forms of PSMA. The inability of the ELISA to uncover such differences suggests that rsPSMA adopts a partially denatured conformation upon adsorption to plastic.

### Example 32

### mAbs 006 and 026 Mediate Efficient ADCC of Human Prostate Cancer Cells

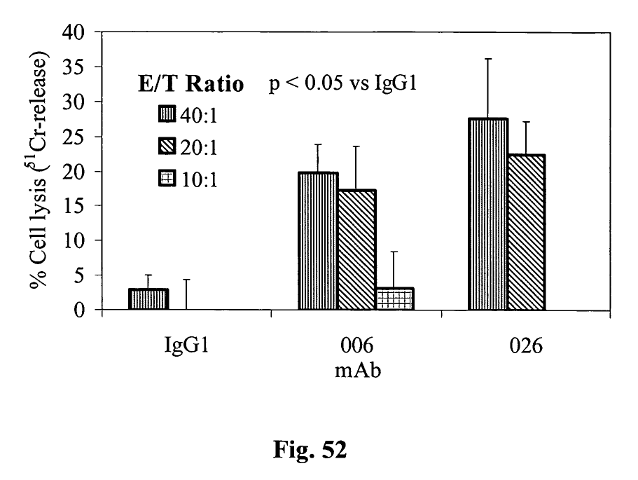

⁵¹Cr labeled C4-2 cells (1×10⁴/well, target cells) were incubated in triplicates with 10 μg/mL mAb at 4° C. for 1 hour. Fresh human PBMCs (effector cells) were added to washed target cells at effector to target (E/T) ratios of 40:1, 20:1, and 10:1 and incubated at 37° C. overnight. ⁵¹Cr in harvested supernatants was measured using a γ-scintillation counter and % cell lysis was calculated. mAbs 006 and 026 demonstrated statistically significant antibody dependent cell-mediated cytotoxicity (ADCC) of C4-2 cells compared to isotype matched human IgG1 mAb control (FIG. 52). No effect was observed when PSMA-negative human prostate tumor cells (PC-3) were used.

### Example 33

### Monomer-Dimer Equilibrium

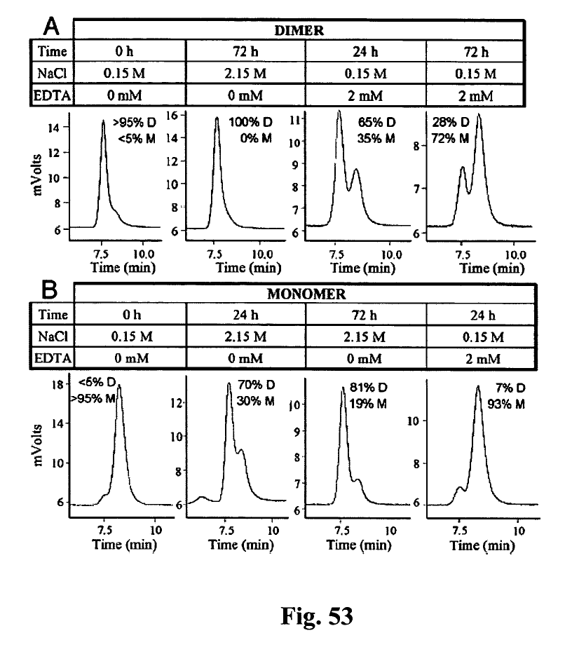

Purified dimeric and monomeric forms of rsPSMA were resolved by preparative size exclusion chromatography (SEC) in PBS+ buffer and collected in separate fractions. To assess whether dimer and monomer exist in a reversible equilibrium, the buffer conditions were perturbed, and the monomer-dimer ratio was analyzed by SEC. As indicated in FIG. 53A, a dimer preparation that contained approximately 5% monomer initially was converted to 100% dimer upon incubation for 72 h at ambient temperature in PBS+ supplemented with 2M sodium chloride (FIG. 53A). Conversely, the addition of 2 mM of the metal-chelating agent EDTA converted the dimer into monomer with a half-life of approximately 2 days (FIG. 53A), indicating that dimer stability is dependent upon the presence of metal ions, such as Zn²⁺ in the active site of PSMA.

For a preparation that initially comprised >95% monomer, high salt similarly drove the equilibrium to mostly (81%) dimer within 72 h (FIG. 53B). EDTA had little influence on the oligomeric state of the monomer. Thus, regardless of the initial oligomeric state of the protein, high salt concentrations promoted dimerization, whereas metal-chelating agents dissociated dimers into monomers.

PSMA shares modest sequence and structural homology with human transferrin receptor (TfR), which contains a vestigial catalytic domain but lacks enzymatic activity. TfR is expressed as a type II membrane protein that forms a disulfide-linked homodimer, but the intermolecular disulfides are not required for dimerization (Alvarez, E., et al. (1989) EMBO J. 8, 2231-2240.0). The high-resolution crystal structure of the TfR ectodomain reveals that the protein is organized into three distinct domains known as the protease-like, apical, and helical domains, with the last domain being principally responsible for dimerization (Lawrence, C. M., et al. (1999) Science 286, 779-782). PSMA and TfR share 30%, 30%, and 24% sequence identity within these domains, respectively. The helical dimerization domain of PSMA are amino acids 601-750 of SEQ ID NO: 1.

### Example 34

### rsPSMA Formulation Studies

pH Stability of rsPSMA

Dimeric rsPSMA (2 mg/ml in PBS+) was diluted 10-fold into a broad-range base buffer solution (2 mM glycine, 2 mM citric acid, 2 mM Hepes, 2 mM MES, 2 mM Tris Base) that was adjusted to cover pH 4 to pH 8.5 in steps of 0.5 pH units. Following incubation for 4 days at 45° C., the individual samples were subjected to analytical TSK gel filtration chromatography (run at pH 7.5) and analyzed for protein recovery and the preservation of the dimeric structure of rsPSMA. The findings are summarized in Table 8.

TABLE 8
Recovery and Structure of rsPSMA at Various pHs

| | Dimer | Monomer | Aggregate | Recovery from |
|---|---|---|---|---|
| pH | Content¹ | Content¹ | Content¹ | column² |
| 4.0 | +++++ | − | − | + |
| 4.5 | − | − | − | − |
| 5.0 | ++ | − | +++ | ++ |
| 5.5 | ++++ | + | − | +++ |
| 6.0 | +++++ | − | − | +++++ |
| 6.5 | ++++ | + | − | ++++ |
| 7.0 | ++++ | + | − | ++++ |
| 7.5 | ++++ | + | − | + |
| 8.0 | +++ | + | + | + |
| 8.5 | − | − | − | − |

¹of recovered protein
²of total protein at t = 0
− <5%
+ 5%-25%
++ 25-50%
+++ 50-75%
++++ 75-95%
+++++ >95%

Base Buffer Evaluation

Dimeric rsPSMA (2 mg/ml in PBS+) was diluted 10-fold into the following buffer solutions:

PBS+

20 mM Hepes, pH 7.0

20 mM sodium phosphate+150 mM NaCl, pH 6.5

20 mM histidine+150 mM NaCl, pH 6.0

20 mM sodium phosphate+150 mM NaCl, pH 6.0

20 mM sodium acetate+150 mM NaCl, pH 6.0

20 mM sodium citrate+150 mM NaCl, pH 6.0

Each sample was incubated for 3 or 4 days at 45° C. and subsequently analyzed by analytical TSK gel filtration chromatography for protein recovery and the preservation of the dimeric structure of rsPSMA. The findings are summarized in Table 9.

TABLE 9
Recovery and Structure of rsPSMA with Various Buffers

| Base | Dimer | Monomer | Aggregate | Recovery from |
|---|---|---|---|---|
| Buffer | Content¹ | Content¹ | Content¹ | column² |
| PBS+ | +++ | − | + | ++ |
| Phosphate | +++++ | − | − | +++++ |
| Acetate | +++++ | − | − | +++++ |
| Citrate | N/A | N/A | N/A | − |
| Histidine | +++ | + | ++ | + |

¹of recovered protein
²of total protein at t = 0
− <5%
+ 5%-25%
++ 25-50%
+++ 50-75%
++++ 75-95%
+++++ >95%

Excipients

Dimeric rsPSMA (2 mg/ml in PBS+) was dialyzed over night into 20 mM sodium acetate, pH 6.0 and 150 mM NaCl. To evaluate the effect of the individual amino acids, the protein was diluted 8-fold into 20 mM sodium acetate, pH 6.0 and 150 mM NaCl containing 50 mM of either glycine, histidine, proline, isoleucine, leucine, alanine, lysine, arginine, threonine, glutamic acid, or aspartic acid as excipients. Following incubation for 5 days at 45° C., each sample was analyzed by analytical TSK gel filtration chromatography for protein recovery and the preservation of the dimeric structure of the protein. The findings are summarized in Table 10.

TABLE 10
Recovery and Structure of rsPSMA with Various Amino Acids

| | Dimer | Monomer | Aggregate | Recovery from |
|---|---|---|---|---|
| Amino Acid | Content¹ | Content¹ | Content¹ | column² |
| Glycine | ++++ | + | − | ++++ |
| Histidine | N/A | N/A | N/A | + |
| Proline | ++++ | + | − | ++++ |
| Isoleucine | ++++ | + | − | ++++ |
| Leucine | ++++ | + | − | ++++ |
| Alanine | ++++ | + | − | ++++ |
| Arginine | ++++ | + | − | ++++ |
| Threonine | ++ | N/A | +++ | ++++ |
| Glutamic Acid | − | − | +++++ | ++++ |
| Aspartic Acid | − | − | +++++ | +++ |

¹of recovered protein
²of total protein at t = 0
− <5%
+ 5%-25%
++ 25-50%
+++ 50-75%
++++ 75-95%
+++++ >95%

Surfactants

Dimeric rsPSMA (2 mg/ml in PBS+) was diluted 10-fold into PBS+ containing 0.5% (w/v) of either Triton X-100, dodecylmaltoside, cholic acid, or CHAPS and incubated for 4 days at 4° C. Each sample was subsequently analyzed by analytical TSK gel filtration chromatography for protein recovery and the preservation of the dimeric structure of the protein. The findings are summarized in Table 11.

TABLE 11
Recovery and Structure of rsPSMA with Various Surfactants

| | Dimer | Monomer | Aggregate | Recovery from |
|---|---|---|---|---|
| Surfactant | Content¹ | Content¹ | Content¹ | column² |
| Triton X-100 | ++++ | + | + | ++++ |
| Dodecylmaltoside | ++++ | − | + | +++++ |
| Cholic Acid | ++++ | − | + | +++++ |
| CHAPS | ++++ | + | − | +++++ |

¹of recovered protein
²of total protein at t = 0
− <5%
+ 5%-25%
++ 25-50%
+++ 50-75%
++++ 75-95%
+++++ >95%

Other Excipients

Dimeric rsPSMA (2 mg/ml in PBS+) was diluted 10-fold into PBS+ containing either 1.4 M (35% saturation) ammonium sulfate, 5 mM EDTA, 1 mM DTT, or 10% glycerol and incubated for 4 days at 4° C. Each sample was subsequently analyzed by analytical TSK gel filtration chromatography for protein recovery and the preservation of the dimeric structure of the protein. The findings are summarized in Table 12.

TABLE 12
Recovery and Structure of rsPSMA with Various Excipients

| | Dimer | Monomer | Aggregate | Recovery |
|---|---|---|---|---|
| Excipient | Content¹ | Content¹ | Content¹ | from column² |
| Ammonium Sulfate | ++++ | − | + | +++++ |
| EDTA | ++ | ++++ | − | +++++ |
| DTT | ++++ | + | − | +++++ |
| Glycerol | ++++ | + | − | +++++ |

¹of recovered protein
²of total protein at t = 0
− <5%
+ 5%-25%
++ 25-50%
+++ 50-75%
++++ 75-95%
+++++ >95%

Conversion of Monomers into Dimers

To evaluate the potential of reversing monomeric rsPSMA into dimers, monomeric rsPSMA (2 mg/ml in PBS+) was diluted 10-fold into PBS+ containing either 1.4 M (35% saturation) ammonium sulfate, 2 M NaCl, 1 mM DTT, 5 mM EDTA, or 10% glycerol and incubated for up to 4 days at 4° C. Each sample was subsequently analyzed by analytical TSK gel filtration chromatography for protein recovery and the formation of the dimeric structure of the protein. The findings are summarized in Table 13.

TABLE 13
Conversion of rsPSMA Monomers

| | Dimer | Monomer | Aggregate | Recovery from |
|---|---|---|---|---|
| Excipient | Content¹ | Content¹ | Content¹ | column² |
| Ammonium Sulfate | ++ | +++ | + | +++++ |
| NaCl | +++ | ++ | + | +++++ |
| DTT | + | ++++ | − | +++++ |
| EDTA | − | +++++ | − | +++++ |
| Glycerol | + | ++++ | − | +++++ |

¹of recovered protein
²of total protein at t = 0
− <5%
+ 5%-25%
++ 25-50%
+++ 50-75%
++++ 75-95%
+++++ >95%

Although the invention has been described in detail for the purpose of illustration, it is understood that such detail is solely for that purpose and variations can be made by those skilled in the art without departing from the spirit and scope of the invention which is defined by the following claims.

The contents of all references, patents and published patent applications cited throughout this application are incorporated herein by reference.

1. A composition comprising isolated PSMA protein, wherein at least 5% of the isolated PSMA protein is an isolated PSMA protein multimer.

2. The composition of claim 1, wherein the isolated PSMA protein multimer is an isolated PSMA protein dimer.

3. The composition of claim 2, wherein the isolated PSMA protein dimer comprises a fragment of full-length PSMA (SEQ ID NO: 1).

4. The composition of claim 2, wherein the isolated PSMA protein dimer comprises a fragment of the extracellular portion of PSMA (amino acids 44-750 of SEQ ID NO: 1).

5. The composition of claim 3, wherein the fragment comprises amino acids 58-750 of SEQ ID NO: 1.

6. The composition of claim 3, wherein the fragment comprises amino acids 44-750 of SEQ ID NO: 1.

7. The composition of claim 3, wherein the fragment comprises amino acids 601-750 of SEQ ID NO: 1.

8. The composition of claim 2, wherein at least 25% of the isolated PSMA protein is in the form of an isolated PSMA protein dimer.

9. The composition of claim 2, wherein at least 50% of the isolated PSMA protein is in the form of an isolated PSMA protein dimer.

10. The composition of claim 2, wherein at least 75% of the isolated PSMA protein is in the form of an isolated PSMA protein dimer.

11. The composition of claim 2, wherein at least 90% of the isolated PSMA protein is in the form of an isolated PSMA protein dimer.

12. The composition of claim 2, wherein at least 95% of the isolated PSMA protein is in the form of an isolated PSMA protein dimer.

13. The composition of any one of claims 1-12, wherein the composition further comprises at least 0.25 molar equivalents of metal ion to PSMA protein.

14. The composition of claim 13, wherein the composition comprises at least 0.5 molar equivalents of metal ion to PSMA protein.

15. The composition of claim 13, wherein the composition comprises at least 1 molar equivalent of metal ion to PSMA protein.

16. The composition of claim 13, wherein the composition comprises a molar excess of metal ion to PSMA protein.

17. The composition of any one of claims 1-16, wherein the composition is in a liquid or lyophilized form.

18. The composition of any one of claims 1-17, wherein the composition further comprises an adjuvant.

19. The composition of claim 18, wherein the adjuvant is alum, monophosphoryl lipid A, a saponin, an immunostimulatory oligonucleotide, incomplete Freund's adjuvant, complete Freund's adjuvant, montanide, vitamin E, a water-in-oil emulsions prepared from a biodegradable oil, Quil A, a MPL and mycobacterial cell wall skeleton combination, ENHANZYN™, CRL-1005, L-121, alpha-galactosylceramide or a combination thereof.

20. The composition of claim 19, wherein the adjuvant is alum.

21. The composition of any one of claims 1-18, wherein the composition further comprises a cytokine.

22. The composition of any one of claims 1-18 and 21, wherein the composition is sterile.

23. The composition of any one of claims 1-18 and 21, wherein the composition is free of chelating agents.

24. The composition of any one of claims 1-18 and 21, wherein the composition further comprises at least one buffer.

25. The composition of claim 24, wherein the at least one buffer is PBS (phosphate buffered saline), citric acid, sodium citrate, sodium acetate, acetic acid, sodium phosphate, phosphoric acid, sodium ascorbate, tartartic acid, maleic acid, glycine, sodium lactate, lactic acid, ascorbic acid, imidazole, sodium bicarbonate, carbonic acid, sodium succinate, succinic acid, histidine, sodium benzoate, benzoic acid or a combination thereof.

26. The composition of any one of claims 1-18 and 21, wherein the composition further comprises a free amino acid, wherein the free amino acid is naturally occurring or non-naturally occurring.

27. The composition of claim 26, wherein the naturally occurring or non-naturally occurring free amino acid is a non-acidic free amino acid.

28. The composition of claim 27, wherein the non-acidic free amino acid is glycine, proline, isoleucine, leucine, alanine, arginine or a combination thereof.

29. The composition of any one of claims 1-18 and 21, wherein the composition further comprises a surfactant.

30. The composition of claim 29, wherein the surfactant is Tween20, Tween80, Triton X-100, dodecylmaltoside, cholic acid, CHAPS or a combination thereof.

31. The composition of any one of claims 1-18 and 21, wherein the composition further comprises a cryoprotectant, an antioxidant, a preservative or a combination thereof.

32. The composition of claim 31, wherein the cryoprotectant is a sugar, a polyol, an amino acid, a polymer, an inorganic salt, an organic salt, trimethylamine N-oxide, sarcosine, betaine, gamma-aminobutyric acid, octapine, alanopine, strombine, dimethylsulfoxide or ethanol.

33. The composition of claim 32, wherein the sugar is sucrose, lactose, glucose, trehalose or maltose.

34. The composition of claim 32, wherein the polyol is inositol, ethylene glycol, glycerol, sorbitol, xylitol, mannitol or 2-methyl-2,4-pentane-diol.

35. The composition of claim 32, wherein the amino acid is Na glutamate, proline, alpha-alanine, beta-alanine, glycine, lysine-HCl or 4-hydroxyproline.

36. The composition of claim 32, wherein the polymer is polyethylene glycol, dextran or polyvinylpyrrolidone.

37. The composition of claim 32, wherein the inorganic salt is sodium sulfate, ammonium sulfate, potassium phosphate, magnesium sulfate or sodium fluoride.

38. The composition of claim 32, wherein the organic salt is sodium acetate, sodium polyethylene, sodium caprylate, proprionate, lactate or succinate.

39. The composition of claim 31, where the antioxidant is ascorbic acid, an ascorbic acid derivative, butylated hydroxy anisole, butylated hydroxy toluene, alkylgallate, dithiothreitol (DTT), sodium meta-bisulfite, sodium bisulfite, sodium dithionite, sodium thioglycollic acid, sodium formaldehyde sulfoxylate, tocopherol, a tocopherol derivative, monothioglycerol or sodium sulfite.

40. The composition of claim 39, wherein the ascorbic acid derivative is ascorbylpalmitate, ascorbylstearate, sodium ascorbate or calcium ascorbate.

41. The composition of claim 39, wherein the tocopherol derivative is d-alpha tocopherol, d-alpha tocopherol acetate, dl-alpha tocopherol acetate, d-alpha tocopherol succinate, beta tocopherol, delta tocopherol, gamma tocopherol or d-alpha tocopherol polyoxyethylene glycol 1000 succinate.

42. The composition of claim 31, wherein the preservative is benzalkonium chloride, chlorobutanol, parabens, thimerosal, benzyl alcohol or phenol.

43. A composition comprising isolated multimeric PSMA protein, wherein the composition comprises less than 35% of a monomeric PSMA protein.

44. The composition of claim 43, wherein the isolated multimeric PSMA protein is an isolated dimeric PSMA protein.

45. The composition of claim 43 or 44, wherein the composition comprises less than 20% of the monomeric PSMA protein.

46. The composition of claim 45, wherein the composition comprises less than 15% of the monomeric PSMA protein.

47. The composition of claim 46, wherein the composition comprises less than 5% of the monomeric PSMA protein.

48. A composition comprising PSMA protein in a solution that promotes or preserves multimeric association of PSMA protein.

49. The composition of claim 48, wherein the solution that promotes or preserves multimeric association of PSMA protein is a solution that promotes or preserves dimeric association of PSMA protein.

50. The composition of claim 48 or 49, wherein the solution that promotes or preserves dimeric association of PSMA protein has a pH that ranges from 4 to 8.

51. The composition of claim 50, wherein the solution that promotes or preserves dimeric association of PSMA protein has a pH that ranges from 5 to 7.

52. The composition of claim 51, wherein the solution that promotes or preserves dimeric association of PSMA protein has a pH that ranges from 5.5 to 7.

53. The composition of claim 51, wherein the solution that promotes or preserves dimeric association of PSMA protein has a pH of 6.

54. The composition of any one of claims 48-53, wherein the solution that promotes or preserves dimeric association of PSMA protein comprises a salt.

55. The composition of claim 54, wherein the cationic component of the salt is sodium, potassium, ammonium, magnesium, calcium, zinc or a combination thereof, and wherein the anionic component of the salt is chloride, sulfate, acetate or a combination thereof.

56. The composition of claim 55, wherein the salt is sodium chloride, sodium sulfate, sodium acetate or ammonium sulfate.

57. The composition of claim 56, wherein the salt is present at a concentration in the range of 50 mM to 2M.

58. The composition of claim 57, wherein the salt is present at a concentration in the range of 100 mM to 300 mM.

59. The composition of claim 58, wherein the salt is present at a concentration of 150 mM.

60. The composition of claim 57, wherein the composition further comprises an adjuvant.

61. The composition of claim 57, wherein the composition is physiologically acceptable.

62. The composition of any one of claims 48-61, wherein the solution that promotes or preserves dimeric association of PSMA protein comprises metal ions.

63. The composition of claim 62, wherein the metal ions are zinc ions, calcium ions, magnesium ions, cobalt ions, manganese ions or a combination thereof.

64. The composition of claim 63, wherein the metal ions are zinc ions and calcium ions.

65. The composition of claim 64, wherein the zinc ions and calcium ions are present at a concentration in the range of 0.1 mM to 5 mM.

66. The composition of claim 64, wherein the zinc ions are present at a concentration that is lower than the concentration of the calcium ions.

67. The composition of claim 66, wherein the zinc ions are present at a concentration of 0.1 mM and the calcium ions are present at a concentration of 1 mM.

68. The composition of claim 63, wherein the metal ions are magnesium ions.

69. The composition of claim 68, wherein the magnesium ions are present at a concentration in the range of 0.1 mM to 5 mM.

70. The composition of claim 69, wherein the magnesium ions are present at a concentration of 0.5 mM.

71. The composition of any one of claims 48-70, wherein the solution that promotes or preserves dimeric association of PSMA protein is free of chelating agents.

72. The composition of any one of claims 48-70, wherein the composition further comprises at least one buffer.

73. The composition of claim 72, wherein the at least one buffer is PBS (phosphate buffered saline), citric acid, sodium citrate, sodium acetate, acetic acid, sodium phosphate, phosphoric acid, sodium ascorbate, tartartic acid, maleic acid, glycine, sodium lactate, lactic acid, ascorbic acid, imidazole, sodium bicarbonate, carbonic acid, sodium succinate, succinic acid, histidine, sodium benzoate, benzoic acid or a combination thereof.

74. The composition of claim 48-70, wherein the composition further comprises a free amino acid, wherein the free amino acid is naturally occurring or non-naturally occurring.

75. The composition of claim 74, wherein the naturally occurring or non-naturally occurring free amino acid is a non-acidic free amino acid.

76. The composition of claim 75, wherein the non-acidic free amino acid is glycine, proline, isoleucine, leucine, alanine, arginine or a combination thereof.

77. The composition of claim 48-70, wherein the composition further comprises a surfactant.

78. The composition of claim 77, wherein the surfactant is Tween20, Tween80, Triton X-100, dodecylmaltoside, cholic acid, CHAPS or a combination thereof.

79. The composition of claim 48-70, wherein the composition further comprises a cryoprotectant, an antioxidant, a preservative or a combination thereof.

80. The composition of claim 79, wherein the cryoprotectant is a sugar, a polyol, an amino acid, a polymer, an inorganic salt, an organic salt, trimethylamine N-oxide, sarcosine, betaine, gamma-aminobutyric acid, octapine, alanopine, strombine, dimethylsulfoxide or ethanol.

81. The composition of claim 80, wherein the sugar is sucrose, lactose, glucose, trehalose or maltose.

82. The composition of claim 80, wherein the polyol is inositol, ethylene glycol, glycerol, sorbitol, xylitol, mannitol or 2-methyl-2,4-pentane-diol.

83. The composition of claim 80, wherein the amino acid is Na glutamate, proline, alpha-alanine, beta-alanine, glycine, lysine-HCl or 4-hydroxyproline.

84. The composition of claim 80, wherein the polymer is polyethylene glycol, dextran or polyvinylpyrrolidone.

85. The composition of claim 80, wherein the inorganic salt is sodium sulfate, ammonium sulfate, potassium phosphate, magnesium sulfate or sodium fluoride.

86. The composition of claim 80, wherein the organic salt is sodium acetate, sodium polyethylene, sodium caprylate, proprionate, lactate or succinate.

87. The composition of claim 79, where the antioxidant is ascorbic acid, an ascorbic acid derivative, butylated hydroxy anisole, butylated hydroxy toluene, alkylgallate, dithiothreitol (DTT), sodium meta-bisulfite, sodium bisulfite, sodium dithionite, sodium thioglycollic acid, sodium formaldehyde sulfoxylate, tocopherol, a tocopherol derivative, monothioglycerol or sodium sulfite.

88. The composition of claim 87, wherein the ascorbic acid derivative is ascorbylpalmitate, ascorbylstearate, sodium ascorbate or calcium ascorbate.

89. The composition of claim 87, wherein the tocopherol derivative is d-alpha tocopherol, d-alpha tocopherol acetate, dl-alpha tocopherol acetate, d-alpha tocopherol succinate, beta tocopherol, delta tocopherol, gamma tocopherol or d-alpha tocopherol polyoxyethylene glycol 1000 succinate.

90. The composition of claim 79, wherein the preservative is benzalkonium chloride, chlorobutanol, parabens, thimerosal, benzyl alcohol or phenol.

91. The composition of any one of claims 48-70, wherein the composition is stable when stored at −80° C.

92. The composition of any one of claims 48-70, wherein the composition is stable when stored at −20° C.

93. The composition of any one of claims 48-70, wherein the composition is stable when stored at 4° C.

94. The composition of any one of claims 39-58, wherein the composition is stable when stored at room temperature.

95. A composition comprising isolated PSMA protein in a solution that promotes or preserves dimeric association of PSMA protein wherein the solution comprises:
(a) 5 to 20 mM of sodium phosphate, sodium acetate or a combination thereof,
(b) 100 to 300 mM sodium chloride or sodium sulfate, and
(c) 0.1 to 2 mM of at least one metal ion.

96. The composition of claim 95, wherein the solution has a pH in the range of 4 to 8.

97. The composition of claim 96, wherein the solution has a pH in a range of 5 to 7.

98. The composition of claim 97, wherein the solution has a pH in a range of 6 to 6.5.

99. The composition of claim 96, wherein the composition further comprises an adjuvant.

100. The composition of claim 99, wherein the adjuvant is alum.

101. The composition of claim 95, wherein the metal ion is a zinc ion, calcium ion, magnesium ion, cobalt ion, manganese ion or a combination thereof.

102. A method of promoting or preserving dimeric association of PSMA protein in a solution comprising:
obtaining a solution of PSMA protein, and
adjusting the pH to be in the range of 4 to 8.

103. The method of claim 102, wherein the pH is adjusted to be in the range of 5 to 7.

104. The method of claim 103, wherein the pH is adjusted to be in the range of 5.5 to 7.

105. The method of claim 104, wherein the pH is adjusted to be 6.

106. A method of processing a PSMA protein comprising:
contacting the PSMA protein in a solution with a first agent that promotes or preserves dimeric association of PSMA protein in an amount effective to promote or preserve PSMA protein dimer formation.

107. The method of claim 106, wherein the amount effective to promote or preserve PSMA protein dimer formation is enough to promote or maintain at least 5%, 25%, 50%, 75% or 95% of the PSMA protein in PSMA dimer form.

108. The method of claim 106, wherein the first agent that promotes or preserves dimeric association of PSMA protein is a salt, metal ion or a pH adjusting agent.

109. The method of claim 108, wherein the cationic components of the salt is sodium, potassium, ammonium, magnesium, calcium, zinc or a combination thereof, and wherein the anionic component of the salt is chloride, sulfate, acetate or a combination thereof.

110. The method of claim 109, wherein the salt is sodium chloride, sodium sulfate, sodium acetate or ammonium sulfate.

111. The method of claim 109, wherein the salt is present at a concentration in the range of 50 mM to 2M.

112. The method of claim 111, wherein the salt is present at a concentration in the range of 100 mM to 300 mM.

113. The method of claim 111, further comprising combining the PSMA protein solution with an adjuvant or diluent.

114. The method of claim 113, wherein the adjuvant or diluent is combined with the PSMA protein in an amount to dilute the salt concentration to 100 mM to 300 mM.

115. The method of claim 114, wherein the salt concentration is diluted to 150 mM.

116. The method of claim 108, wherein the metal ion is a zinc ion, calcium ion, magnesium ion, cobalt ion, manganese ion or a combination thereof.

117. The method of claim 116, wherein the metal ion is a combination of zinc ion and calcium ion.

118. The method of claim 117, wherein the zinc ion and calcium ion are present at a concentration in the range of 0.1 mM to 5 mM.

119. The method of claim 117, wherein the zinc ion is present at a concentration that is lower than the concentration of the calcium ion.

120. The method of claim 119, wherein the zinc ion is present at a concentration of 0.1 mM and the calcium ion is present at a concentration of 1 mM.

121. The method of claim 116, wherein the metal ion is a magnesium ion.

122. The method of claims 121, wherein the magnesium ion is present at a concentration in the range of 0.1 mM to 5 mM.

123. The method of claim 122, wherein the magnesium ion is present at a concentration of 0.5 mM.

124. The method of any one of claims 108-123, wherein the pH of the solution is adjusted to be in the range of 4 to 8.

125. The method of claim 124, wherein the pH of the solution is adjusted to be in the range of 5 to 7.

126. The method of claim 125, wherein the pH of the solution is adjusted to be in the range of 5.5 to 7.

127. The method of claim 126, wherein the pH of the solution is adjusted to be 6.

128. The method of claim 108, wherein the method further comprises contacting the PSMA protein with a second agent that promotes or preserves dimeric association of PSMA protein, and wherein the second agent is different than the first agent.

129. The method of claim 128, wherein the second agent that promotes or preserves dimeric association of PSMA protein is a metal ion, salt or pH adjusting agent.

130. The method of claim 129, wherein the metal ion is a zinc ion, calcium ion, magnesium ion, cobalt ion, manganese ion or a combination thereof.

131. The method of claim 129, wherein the cationic components of the salt is sodium, potassium, ammonium, magnesium, calcium, zinc or a combination thereof, and wherein the anionic component of the salt is chloride, sulfate, acetate or a combination thereof.

132. The composition of claim 131, wherein the salt is sodium chloride, sodium sulfate, sodium acetate or ammonium sulfate.

133. The method of any one of claims 128-132, wherein the pH of the solution is adjusted to be in the range of 4 to 8.

134. The method of claim 133, wherein the pH of the solution is adjusted to be in the range of 5 to 7.

135. The method of claim 134, wherein the pH of the solution is adjusted to be in the range of 5.5 to 7.

136. The method of claim 135, wherein the pH of the solution is adjusted to be 6.

137. A method of purifying a sample containing PSMA protein comprising:
subjecting the sample containing PSMA to chromatography in the presence of an agent that preserves or promotes the dimeric association of PSMA.

138. The method of claim 137, wherein the agent that promotes or preserves the dimeric association of PSMA is a metal ion, a salt or a solution with a pH in the range of 4 to 8 or a combination thereof.

139. The method of claim 138, wherein the metal ion is a zinc ion, calcium ion, magnesium ion, cobalt ion, manganese ion or a combination thereof.

140. The method of claim 139, wherein the metal ion is a combination of calcium ion and magnesium ion.

141. The method of claim 140, wherein the calcium ion and magnesium ion are each present at a concentration in the range of 0.1 mM to 5 mM.

142. The method of claim 141, wherein the calcium ion and magnesium ion are present at a concentration of 1 mM and 0.5 mM, respectively.

143. The method of claim 138, wherein the cationic component of the salt is sodium, potassium, ammonium, magnesium, calcium, zinc or a combination thereof, and wherein the anionic component of the salt is chloride, sulfate, acetate or a combination thereof.

144. The method of claim 143, wherein the salt is sodium chloride, sodium sulfate, sodium acetate or ammonium sulfate.

145. The method of claim 143, wherein the salt is present at a concentration in the range of 50 mM to 2M.

146. The method of claim 145, wherein the salt is present at a concentration of 2M.

147. The method of claim 138, wherein the pH of the solution is in the range of 5 to 7.

148. The method of claim 147, wherein the pH of the solution is maintained in the range of 6 to 7.5.

149. A method of purifying a sample containing PSMA protein comprising:
applying the sample to a first column,
washing the first column with a first wash solution containing salt and metal ions, and collecting the PSMA protein that elutes from the first column.

150. The method of claim 149, wherein the metal ions are zinc ions, calcium ions, magnesium ions, cobalt ions, manganese ions or a combination thereof.

151. The method of claim 150, wherein the metal ions are calcium and magnesium ions.

152. The method of claim 151, wherein the calcium ions and magnesium ions are present at a concentration in the range of 0.1 mM to 5 mM.

153. The method of claim 152, wherein the calcium ions and magnesium ions are present at a concentration of 1 mM and 0.5 mM, respectively.

154. The method of claim 152, wherein the cationic component of the salt is sodium, potassium, ammonium, magnesium, calcium, zinc or a combination thereof, and wherein the anionic component of the salt is chloride, sulfate acetate or a combination thereof.

155. The method of claim 154, wherein the salt is ammonium sulfate at a saturation of no more than 35% in the wash solution.

156. The method of claim 149, further comprising dialyzing or diafiltering the eluted PSMA protein with a first salt solution at a pH in the range of 6 to 7.5 to yield a dialyzed or diafiltrated solution containing PSMA protein.

157. The method of claim 156, wherein the first salt solution has a salt concentration of at least 5 mM.

158. The method of claim 157, wherein the first salt solution is a 10M sodium phosphate solution with a pH of 7.

159. The method of claim 149 or 156, further comprising:
loading the eluted PSMA protein, dialyzed or diafiltrated solution containing PSMA protein onto a second column,
washing the second column with a second salt solution,
and collecting the PSMA eluted by the second salt solution.

160. The method of claim 159, wherein the second salt solution has a salt concentration of 100 mM to 2M.

161. The method of claim 160, wherein the second salt solution is 2M sodium chloride in 10 mM sodium phosphate.

162. The method of claim 159 or 160, wherein the second salt solution has a pH in the range of 6 to 7.5.

163. The method of claim 159, further comprising
dialyzing or diafiltrating the PSMA eluted by the second salt solution with a metal ion solution,
applying the dialyzed or diafiltrated PSMA eluted by the second salt solution onto a third column,
washing the third column with a second wash solution containing salt and metal ions and collecting the PSMA eluted.

164. The method of claim 163, wherein the pH is maintained in the range of 6 to 7.5 through all of the purification steps.

165. The method of claim 163, further comprising separating the different forms of PSMA protein, wherein the different forms of PSMA protein are monomeric, dimeric or other multimeric forms of PSMA.

166. The method of claim 165, wherein the different forms of PSMA protein are separated by size exclusion chromatography.

167. A method of identifying an agent which promotes or preserves dimeric association of PSMA protein comprising:
determining the amount of a form of PSMA protein in a sample prior to exposure to a candidate agent,
exposing the sample to the candidate agent,
determining the amount of the form of PSMA protein in the sample after the exposure, and
comparing the amount of the form of PSMA protein in the sample prior to and after the exposure.

168. The method of claim 167, wherein the form of PSMA protein is monomer or dimer.

169. A method of treating a subject to elicit or enhance an immune response to cells in the subject expressing PSMA, comprising administering to the subject an effective amount of the composition of any one of claims 1-22, 43-71 and 95.

170. The method of claim 171, wherein the expressed PSMA is expressed on the cell surface.

171. The method of claim 169, wherein the method further comprises administering one or more booster doses of a composition comprising PSMA protein.

172. The method of claim 171, wherein the composition comprising PSMA protein is a composition of PSMA protein dimer.

173. The method of claim 171, wherein the booster dose composition further comprises an adjuvant.

174. The method of claim 171, wherein the booster dose composition is the composition of any one of claim 1-22, 43-71 and 95.

175. The method of claim 169, wherein the composition is administered by intravenous, intramuscular, subcutaneous, parenteral, spinal, intradermal or epidermal administration.

176. The method of claim 175, wherein the composition is administered by subcutaneous administration.

177. The method of claim 169, wherein the subject has cancer or has been treated for cancer.

178. The method of claim 177, wherein the cancer is a primary tumor or is metastatic cancer.

179. The method of claim 177, wherein the subject has prostate cancer.

180. A method of eliciting an immune response, comprising administering to a subject an effective amount of the composition of any one of claims 1-22, 43-71 and 95.

181. The method of claim 180, wherein the method further comprises administering one or more booster doses of a composition comprising PSMA protein.

182. The method of claim 181, wherein the composition comprising PSMA protein is a composition PSMA protein dimer.

183. The method of claim 181, wherein the booster dose composition further comprises an adjuvant.

184. The method of claim 181, wherein the booster dose composition is the composition of any one of claim 1-22, 43-71 and 95.

185. The method of claim 180, wherein the composition is administered by intravenous, intramuscular, subcutaneous, parenteral, spinal, intradermal or epidermal administration.

186. The method of claim 185, wherein the composition is administered by subcutaneous administration.

187. A kit which comprises the composition of any one of claims 1-22, 43-71 and 95 and instructions for use.

188. A kit which comprises the composition of any one of claim 22, 43-58, 39-49, 62-71 and 95, an adjuvant and instructions for mixing.

189. The kit of claim 188, wherein the adjuvant is alum.

190. A kit which comprises the composition of any one of claims 22, 43-58, 39-49, 62-71 and 95, a diluent and instructions for mixing.

191. The kit of any one of claims 187, 188, and 189, wherein the composition is provided in a vial or ampoule with a septum or a syringe.

192. The kit of any one of claims 187, 188, and 189, wherein the composition is in lyophilized form.

193. A pharmaceutical composition comprising the composition of any one of the compositions of claims 1-22, 43-71 and 95, and a pharmaceutically acceptable carrier.
